(19) United States
(12) Patent Application Publication
(10) Pub. No.: US 2025/0099625 A1
(43) Pub. Date: Mar. 27, 2025

(54) TANDEM MOLECULAR FLUORESCENCE REPORTERS FOR DETECTION OF TUMOR-INFILTRATING LEUKOCYTES

(71) Applicant: Nanyang Technological University, Singapore (SG)

(72) Inventors: Kanyi PU, Singapore (SG); Shasha HE, Singapore (SG)

(21) Appl. No.: 18/712,544

(22) PCT Filed: Dec. 13, 2022
(86) PCT No.: PCT/SG2022/050903

§ 371 Date: May 22, 2024

(30) Foreign Application Priority Data
Dec. 14, 2021 (SG) 10202113855P

(51) Int. Cl. A61K 49/00; A61K 47/65; G01N 33/50

(52) U.S. Cl. CPC A61K 49/0032; A61K 47/65; A61K 49/0056; G01N 33/5011

(57) ABSTRACT

The current invention relates to a compound of formula (I): where X− represents a counterion and A represents an amino acid moiety that is cleavable by an enzyme associated with a leukocyte, or a pharmaceutically acceptable salt or solvate thereof.

### FIELD OF INVENTION

The current invention relates to diagnostic molecules that can be used in a subject or in vitro to determine whether a subject has a particular disease or whether that disease is susceptible to particular treatments. The invention also relates to methods of making said compounds and the compounds applied to the uses mentioned above.

### BACKGROUND

The listing or discussion of a prior-published document in this specification should not necessarily be taken as an acknowledgement that the document is part of the state of the art or is common general knowledge.

Cancer immunotherapy that trains the immunity to eradicate cancer has revolutionized the landscape of oncology. However, patients often respond differently to the same immunotherapy, and the response rates toward checkpoint blockade therapy remain low in many cancer types (20-40% melanoma, renal cell carcinoma, and colorectal cancer). Tumor-infiltrating leukocytes (TILs) are known to be closely associated with the cancer progression and clinical endpoints of cancer patients. Particularly, clinical data has revealed that high levels of M1 macrophages and cytotoxic T lymphocytes (CTLs) were associated with positive prognosis (Fridman, W. H. et al., Nat. Rev. Clin. Oncol. 2017, 14, 717-734), while increased neutrophil-lymphocyte ratio predicted poor survival of cancer patients (Gentles, A. J. et al., Nat. Med. 2015, 21, 938-945). To assess TILs in tumor immune microenvironment (TIME) for patient stratification and therapeutic predication before and during cancer immunotherapy, flow cytometry, histological staining, and mass cytometry of biopsy tumor tissues have been used. However, single-site biopsy is invasive, static, risky to cause metastasis and is only able to reveal regional information on TIME; whereas, peripheral blood analysis inevitably contains biomarkers secreted from organs other than tumor, reducing the specificity towards TIME.

Molecular imaging offers a real-time and non-invasive way for longitudinal monitoring of overall TIME. However, existing imaging modalities including computed tomography (CT), positron emission tomography (PET) and magnetic resonance imaging (MRI) fail to accurately monitor of TILs in TIME, because they rely on antibody/ligand conjugated contrast agents to target the leukocytes of interest, and their “always-on” signals are inevitably compounded by nonspecific retention in tissues other than tumor. In contrast, activatable molecular optical reporters only trigger their signals against intended biomarkers and thus have minimized background and quantitative signals with the concentration and activity of biomarkers. Recently, activatable molecular reporters have been developed for real-time imaging of leukocytes; however, as their activation mechanism is solely determined by the leukocyte biomarkers, their signals can be nonspecifically triggered by the leukocytes in peripheral blood and inflammatory tissues. Thus, specific real-time non-invasive imaging of TILs remains a great challenge.

Therefore, there exists a need for new molecular fluorescence reports for detection of TILS.

### SUMMARY OF INVENTION

Aspects and embodiments will now be discussed by reference to the following clauses.

1. A compound of formula I:

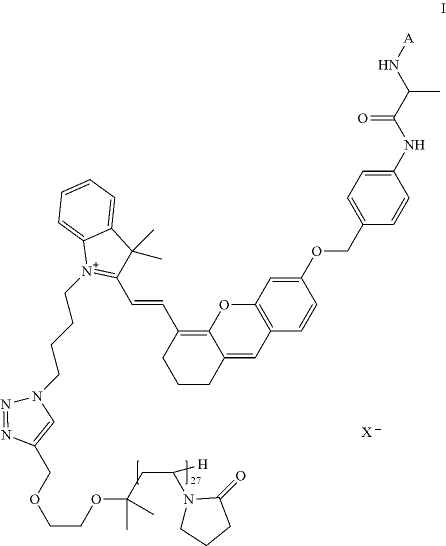

wherein:
X⁻ represents a counterion; and
A represents an amino acid moiety that is cleavable by an enzyme associated with a leukocyte,
or a pharmaceutically acceptable salt or solvate thereof.

2. The compound according to Clause 1, or a salt or solvate thereof, wherein A is selected from:

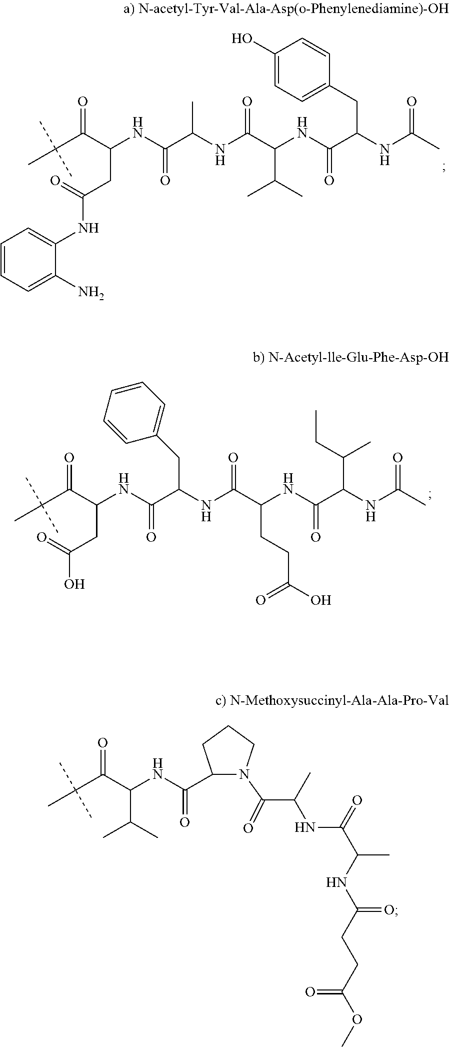

where the point of attachment is denoted by the dotted line.

3. Use of a compound of formula I as defined in Clause 1 or Clause 2, or a salt or solvate thereof in the preparation of an imaging agent for the diagnosis of a condition or disease in a tissue and/or an organ using near-infrared fluorescence.

4. A compound of formula I as defined in Clause 1 or Clause 2, or a salt or solvate thereof for use as an imaging agent for the diagnosis of a condition or disease in a tissue and/or an organ using near-infrared fluorescence.

5. The use according to Clause 3 or the compound for use according to Clause 4, wherein the use is in vivo or in vitro.

6. The use or compound for use according to Clause 5, wherein the in vivo imaging is for the purpose of visualizing the tumour immune microenvironment.

7. A method of diagnosing a condition or disease in a tissue and/or an organ, the method comprising the steps of administering a compound of formula I as defined in Clause 1 or Clause 2, or a salt or solvate thereof to a subject in need of diagnosis and determining the presence or absence of a condition or disease in a tissue and/or an organ using near-infrared fluorescence.

8. A method of determining the susceptibility of a tumour tissue to an immunotherapy, the method comprising the steps of:
(i) subjecting a tumour tissue to a desired course of immunotherapy;
(ii) administering a compound of formula I as defined in Clause 1 or Clause 2, or a salt or solvate thereof to the tumour tissue along with an always on reference reporter compound;
(iii) after a period of time determining the ratio of near-infrared fluorescence obtained from a metabolite of the compound of formula I as defined in Clause 1 or Clause 2 relative to the always on reference reporter compound; and
(iv) determining the susceptibility of the tumour tissue to the immunotherapy based on the ratio obtained in step (iii).

9. The method according to Clause 8, wherein the method is conducted in vivo.

10. The method according to Clause 8, wherein the method is conducted in vitro.

11. The method according to any one of Clauses 8 to 10, wherein the method further involves subjecting a subject to a therapeutic treatment regimen based on the results obtained by the method.

12. The method according to any one of Clauses 8 to 11, wherein the always on reference reporter compound is:

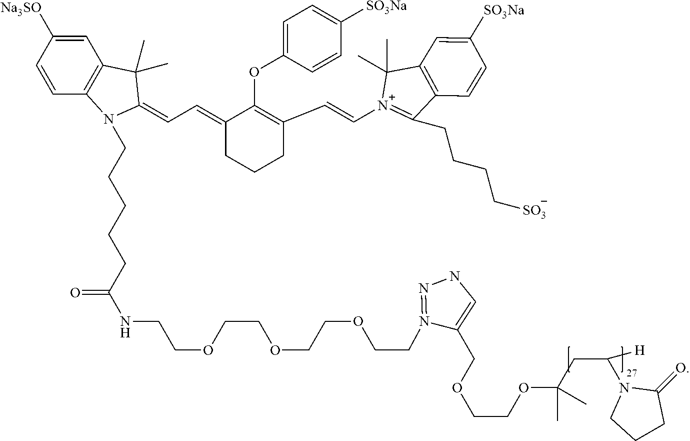

### DRAWINGS

FIG. 1 depicts the design and mechanism of tandem-activatable molecular reporters (TAMRs) for specific molecular optical imaging of TILs. (a) Schematic illustration of targeted TILs in tumor, real-time, non-invasive and specific imaging of TILs during cancer immunotherapy using TAMRs, and the potential applications of TAMRs for patient stratification and prediction of therapeutic efficacy. (b) Chemical structures of TAMRs including TAMR_{M1}, TAMR_{CTL}, and TAMR_{NE} and their activation process in the presence of cancer and leukocyte biomarkers, and chemical structure of tumor penetrating reference reporter, polyvinylpyrrolidone (PVP)-IR800. (c) Schematic illustration of TAMRs for monitoring of TILs. Upon TAMRs accumulation into TIME, nitric oxide (NO) and activated caspase-1 (Cas-1) overexpressed by tumor-infiltrating M1 macrophages, Granzyme B (GrB) overexpressed by tumor-infiltrating CTLs, and NE overexpressed by tumor-infiltrating neutrophils, will cleave their respective peptide substrates (R₂ segments) of TAMRs, and tumor biomarker (APN) can subsequently attack the exposed alanine (R₁ segment), resulting in the fluorescence signal activation of TAMRs.

FIG. 2 depicts the synthesis of TAMRs. (a) Synthesis of fluorescent signaling moiety (CyOH) and tumor-passive targeting moiety (PVP-alkyne): (i) resorcinol, K₂CO₃, acetonitrile (CH₃CN), 55° C., 6 h; (ii) isopropoxyethanol, 2,2′-azobisisobutyronitrile (AIBN), 60° C., 4 h; and (iii) propargyl bromide, tetrahydrofuran (THF), 25° C., 24 h. (b) Synthesis of CyA: (i) p-aminobenzyl alcohol (PABA), N-ethoxycarbonyl-2-ethoxy-1,2-dihydroquinoline (EEDQ), dichloromethane (DCM), 25° C., 6 h; (ii) PBr₃, THF, 0° C., 2 h; (iii) CyOH, N,N-diisopropylethylamine (DIPEA), CH₃CN, 55° C., 8 h; and (iv) piperidine, dimethylformamide (DMF), 25° C., 5 min. (c) Synthesis of TASMRs: (i) Ac-Y(tBu)VAD(OtBu)—OH, hexafluorophosphate benzotriazole tetramethyl uronium (HBTU), hydroxybenzotriazole (HOBt), DIPEA, DMF, 25° C., 2 h; (ii) trifluoroacetic acid (TFA)/H₂O, 0° C., 40 min; (iii) o-phenylenediamine (OPD), HBTU, HOBt, DIPEA, DMF, 25° C., 0.5 h; (iv) Ac-IE(OtBu)FD(OtBu)—OH, HBTU, HOBt, DIPEA, DMF, 25° C., 2 h; (v) TFA/H₂O, 0° C., 40 min; and (vi) MeOSuc-AAPV-OH, HBTU, HOBt, DIPEA, DMF, 25° C., 2 h. (d) Synthesis of TAMRs: (i) PVP-alkyne, CuSO₄·5H₂O, sodium ascorbate, H₂O/dimethyl sulfoxide (DMSO), 25° C., 12 h.

FIG. 3 depicts the pharmacokinetics of (a) polyethylene glycol (PEG)-Cy and (b) PVP-Cy in healthy mice after intravenous (i.v.) injection of the two probes. Healthy mice were intravenously injected with the uncaged PEG-Cy and PVP-Cy. [Cy]=5 μmol/kg. The circulatory half-life of PVP-Cy and PEG-Cy was calculated to be 48 min and 11 min, respectively. (c) Real-time imaging of CT26 tumor-bearing mice after i.v. injection of PEG-Cy or PVP-Cy (5 μmol/kg). NIRF images were acquired at 720 nm with excitation at 675 nm by an IVIS spectrum imaging system. (d) Quantification of NIRF signals at tumor sites as a function of post-injection timepoints of probes. The NIRF signals in the tumor regions gradually increased and reached maxima at 6 h and 8 h post-injection of PVP-Cy and PEG-Cy, respectively. The error bars represent the standard deviation of three separate measurements.

FIG. 4 depicts the ultraviolet-visible (UV-Vis) absorption spectra of (a) TAMR_{M1}, (b) TAMR_{CTL}, and (c) TAMR_{NE} in the presence of individual biomarker or combined biomarkers in corresponding buffers at 37° C. Note that GrB (0.5 μg) was first activated by cathepsin C (0.2 μg) in MES buffer for 4 h. APN buffer: 50 mM tris, pH 7.0. For TAMR_{M1}, DEA NONOate (50 μM) was used as the source of NO. [TASMR]=25 μM. All the enzymatic experiments were incubated at 37° C. for 2 h and repeated independently three times with similar results.

FIG. 5 depicts the in vitro characterization of the detection capabilities of TAMRs. Fluorescence spectra of (a) TAMR_{M1}, (b) TAMR_{CTL}, and (c) TAMR_{NE} in the presence of individual biomarker or combined biomarkers in corresponding buffers at 37° C. for 2 h. [TAMRs]=25 μM. Excitation: 680 nm. (d-f) In vitro selectivity of TAMRs. Near-infrared fluorescence (NIRF) changes of (d) TAMR_{M1}, (e) TAMR_{CTL}, and (f) TAMR_{NE} at 720 nm after incubation with individual biomarker or combined biomarkers in corresponding buffers at 37° C. for 2 h (n=3). [TAMRs]=25 μM. (d) 1: Blank; 2: urokinase-type plasminogen activator (uPA); 3: nitroreductase (NTR); 4: gamma-glutamyltransferase (GGT); 5: fibroblast activation protein (FAP); 6: caspase-3; 7: cathepsin S (CTSS); 8: GrB; 9: NE; 10: CTSS&APN; 11: GrB&APN; 12: NE&APN; 13: DPPI; 14: NO&Cas-1; 15: APN; and 16: NO&Cas-1&APN. (e) 1: Blank; 2: uPA; 3: NTR; 4: GGT; 5: FAP; 6: caspase-3; 7: NO&Cas-1; 8: CTSS; 9: NE; 10: NO&Cas-1&APN; 11: CTSS&APN; 12: NE&APN; 13: DPPI; 14: GrB; 15: APN; and 16: GrB&APN. (f) 1: Blank; 2: uPA; 3: NTR; 4: GGT; 5: FAP; 6: caspase-3; 7: NO&Cas-1; 8: GrB; 9: CTSS; 10: NO&Cas-1&APN; 11: GrB&APN; 12: CTSS&APN; 13: DPPI; 14: NE; 15: APN; and 16: NE&APN. 16 versus other groups: TAMR_{M1}: P<0.0001, TAMR_{CTL}: P<0.0001, TAMR_{NE}: P<0.0001. (g, h) Confocal imaging and quantification of TAMRs in cells: (g) Confocal imaging and (h) corresponding mean fluorescence intensities of M1 macrophages, CD8 T cells and neutrophils after incubation with TAMR_{M1}, TAMR_{CTL}, and TAMR_{NE} in the presence or absence of APN (0.5 μg) for 2 h. [TAMRs]=10 μM. All confocal imaging experiments were repeated independently three times. Data are presented as mean±SD and analyzed by two-tailed Student's t-test.

FIG. 6 depicts the UV-Vis absorption spectra of (a) TASMR_{M1}, (b) TASMR_{CTL}, and (c) TASMR_{NE} and fluorescence spectra of (d) TASMR_{M1}, (e) TASMR_{CTL}, and (f) TASMR_{NE} in the presence of individual biomarker or combined biomarkers (50 μM DEA NONOate, 1 U Cas-1, 0.5 μg APN, 0.5 μg GrB, 2.5 mU NE) in corresponding buffers at 37° C. The excitation wavelength for fluorescence spectra was 680 nm. All the enzymatic experiments were incubated at 37° C. for 2 h and repeated independently three times with similar results. [TASMR]=25 μM. In the presence of both tumor (APN) and M1 macrophage biomarkers (Cas-1 and NO), the fluorescence signal of TASMRM1 at 710 nm was increased by 28-fold. In the presence of both tumor (APN) and CTL biomarkers (GrB), the fluorescence signal of TASMRCTL at 710 nm was increased by 15-fold. In the presence of both tumor (APN) and neutrophil biomarkers (NE), the fluorescence signal of TASMR_{NE} at 710 nm was increased by 10-fold.

FIG. 7 depicts the NIRF changes of (a) TASMR_{M1}, (b) TASMR_{CTL}, and (c) TASMR_{NE} at 720 nm after incubation with individual biomarker or combined biomarkers in corresponding buffers at 37° C. for 2 h. (a) 1: Blank; 2: uPA; 3: NTR; 4: GGT; 5: FAP; 6: caspase-3; 7: CTSS; 8: GrB; 9: NE; 10: CTSS&APN; 11: GrB&APN; 12: NE&APN; 13: DPPI; 14: NO&Cas-1; 15: APN; and 16: NO&Cas-1&APN. (b) 1: Blank; 2: uPA; 3: NTR; 4: GGT; 5: FAP; 6: caspase-3; 7: NO&Cas-1; 8: CTSS; 9: NE; 10: NO&Cas-1&APN; 11: CTSS&APN; 12: NE&APN; 13: DPPI; 14: GrB; 15: APN; and 16: GrB&APN. (c) 1: Blank; 2: uPA; 3: NTR; 4: GGT; 5: FAP; 6: caspase-3; 7: NO&Cas-1; 8: GrB; 9: CTSS; 10: NO&Cas-1&APN; 11: GrB&APN; 12: CTSS&APN; 13: DPPI; 14: NE; 15: APN; and 16: NE&APN. [TASMR]=25 μM. All the enzymatic experiments were repeated independently three times with similar results. TASMRs showed negligible enhancement after incubation with other interfering enzymes.

FIG. 8 depicts the high performance liquid chromatography (HPLC) traces of (a) TASMR_{M1}, (b) TASMR_{CTL} and (c) TASMR_{NE} in the absence or presence individual biomarker or combined biomarkers in corresponding buffers (50 μM DEA NONOate, 1 U Cas-1, 0.5 μg APN, 0.5 μg GrB, 2.5 mU NE) at 37° C. for 2 h. [TASMR]=25 μM. HPLC analysis of the pure CyA and CyOH was used for comparison. HPLC traces of all intermediate enzymatic products were repeated independently three times with similar results.

FIG. 9 depicts the nonlinear regression analysis of cleavage rate (V) of (a) TASMR_{M1} by Cas-1, (b) TASMR_{CTL} by GrB, (c) TASMR_{NE} by NE and (d) CyA by APN as a function of substrate concentration. Various concentrations of TASMRs and CyA were incubated with respective enzymes at 37° C.

FIG. 10 depicts the cell viability of CD8 T cells, M1 macrophages, neutrophils, 4T1 and CT26 cells after treatment with (a) TAMR_{M1}, (b) TAMR_{CTL} and (c) TAMR_{NE} for 24 h at final concentrations from 12.5 to 100 μg/mL.

FIG. 11 depicts the representative confocal fluorescence imaging of 3T3 cells (mouse embryonic fibroblast cell line) after incubation with TAMR_{M1}, TAMR_{CTL}, and TAMR_{NE} in the presence or absence of APN (0.5 μg) for 2 h. [TAMR]=10 μM.

FIG. 12 depicts the representative confocal fluorescence imaging of M0 phenotype of RAW264.7 cells (murine macrophages) after incubation with TAMR_{M1}, TAMR_{CTL}, and TAMR_{NE} in the presence or absence of APN (0.5 μg) for 2 h. [TAMR]=10 μM.

FIG. 13 depicts the representative confocal fluorescence imaging of M2 phenotype of RAW264.7 cells after incubation with TAMR_{M1}, TAMR_{CTL}, and TAMR_{NE} in the presence or absence of APN (0.5 μg) for 2 h. [TAMR]=10 μM.

FIG. 14 depicts the representative confocal fluorescence imaging of bone marrow-derived dendritic cells (BMDCs) after incubation with TAMR_{M1}, TAMR_{CTL}, and TAMR_{NE} in the presence or absence of APN (0.5 μg) for 2 h. [TAMR]=10 μM.

FIG. 15 depicts the representative confocal fluorescence imaging of 4T1 and CT26 cells after incubation with TAMR_{M1}, TAMR_{CTL}, and TAMR_{NE} for 2 h. [TAMR]=10 μM.

FIG. 16 depicts the superiority of TAMRs in comparison to single-locked activatable molecular reporters (AMRs). (a) Chemical structures of AMRs including AMR_{M1}, AMR_{CTL} and AMR_{NE}, and their activated forms in inflammatory leukocytes and TILs. (b, c) Comparison of TAMRs and AMRs in blood samples: (b) scheme for the experimental procedures for comparison of TAMRs and AMRs in the blood samples from saline or lipopolysaccharides (LPS)-treated mice; and (c) NIRF signals of TAMRs and AMRs (10 μM) after incubation in the blood samples from saline or LPS-treated mice for 30 min (n=3). Black line indicated the NIRF intensity of the same concentration of these reporters in phosphate buffered saline (PBS). Saline versus LPS-treated groups: not significant (ns). (d-f) Comparison of TAMRs and AMRs in living mice: (d) schematic illustration of comparison of TAMRs and AMRs in living mice bearing LPS-inflamed tissue in the left thigh muscle and subcutaneous CT26 tumor on the right flank; and (e) quantification of the NIRF signals and (f) NIRF images of reporters (0.1 mM, 10 μL) in the LPS-inflammed and CT26 tumor tissues after local injection of reporters for 30 min (n=3). Black line indicates the background NIRF intensities of these reporters in the skin. Tumor versus LPS-inflamed tissues, TAMR_{M1}: P<0.0001, TAMR_{CTL}: P<0.0001, TAMR_{NE}: P=0.0001, AMRs: ns. (g-j) Comparison of TAMRs and AMRs via urinalysis: (g) schematic illustration of fluorescence urinalysis process; (h) renal clearance efficiency of TAMRs and AMRs in healthy mice after 24 h i.v. injection of these reporters (n=3); and (i) NIRF images and (j) quantification of the NIRF signals of reporters in the urine collected from healthy mice at 12 h post-injection of TAMRs or AMRs. Black line indicates the NIRF intensity of the same concentration of these reporters in PBS (n=3). TAMR_{M1} versus AMR_{M1}: P=0.0006; TAMR_{CTL} versus AMR_{CTL}: P=0.0052; TAMR_{NE} versus AMR_{NE}: P=0.0003. All NIRF images were captured via an IVIS spectrum imaging system. Excitation: 675 nm, Emission: 720 nm. Data are presented as mean±SD and analyzed by two-tailed Student's t-test.

FIG. 17 depicts the absorption intensity at 680 nm of TAMRs or AMRs (10 μM) in the supernatants of homogenized LPS-inflamed and saline-treated blood samples. LPS (5.0 mg/kg) was intraperitoneally injected into living mice, and the LPS-inflamed blood samples were collected 4 h post-injection of LPS. TAMRs and AMRs (10 μM) were incubated with LPS-inflamed and saline-treated blood samples for 30 min. Then, the blood samples were homogenized and centrifuged for 10 min. The supernatants were used for absorption analysis.

FIG. 18 depicts (a) NIRF images of reporters in the LPS-inflammed and CT26 tumor tissues after local injection of TAMRs or AMRs. (b) Quantification of NIRF intensities at LPS-inflammed and CT26 tumor tissues as a function of post-injection timepoints of reporters. CT26 tumors were first inoculated in the right flank for 7 days, then, LPS (5 mg/kg) was injected into the left thigh muscle. 24 h later, 10 μL of TAMRs or AMRs (0.1 mM) were locally injected into both LPS-inflamed and CT26 tumors for longitudinal NIRF imaging. NIRF images were acquired at 720 nm with excitation at 675 nm by an IVIS spectrum imaging system. *P<0.05, **P<0.01, ****P<0.0001. All experiments were repeated independently three times. Statistical significance was performed by two-tailed Student's t-test. ns: not significant, *P<0.05, **P<0.01, ***P<0.001, ****P<0.0001.

FIG. 19 depicts (a) real-time imaging of 4T1 and CT26 tumor-bearing mice after i.v. injection of TAMR_{CTL} (5 μmol/kg) and PVP-IR800 (3 μmol/kg) into the mice in presence or absence of APN inhibitor (bestatin). Bestatin (10 mg/mL, 10 μL) was injected intratumorly 2 h before i.v. injection of TAMR_{CTL}. NIRF images were acquired at 720 nm with excitation at 675 nm by an IVIS spectrum imaging system. (b) Quantification of R-NIRF_{CTL} at tumor sites as a function of post-injection timepoints of reporters. (n=3).

FIG. 20 depicts the renal clearance efficiency of (a) TAMR_{M1}, (b) AMR_{M1}, (c) TAMR_{CTL}, (d) AMR_{CTL}, (e) TAMR_{NE} and (f) AMR_{NE} in healthy mice after i.v. injection of probes (n=3). Healthy mice were intravenously injected with TAMRs (5 μmol/kg) or AMRs (5 μmol/kg), and placed into metabolic cages. Urine samples were collected at 3, 9, and 24 h post-injection of these reporters. The renal clearance of these reporters was examined by HPLC analysis of the urine samples.

FIG. 21 depicts the UV-Vis absorption spectra of probes in the urine collected from healthy mice. The shaded rectangle indicates the appearance of peak at 680 nm of AMRs in the urine.

FIG. 22 depicts the in vivo specific real-time NIRF imaging of TILs in 4T1 tumor-bearing mice. (a) Schematic illustration of immunotherapeutic process and real-time imaging of TILs. (b) Real-time imaging of 4T1 tumor-bearing mice with different treatments after i.v. injection of TAMRs (5 μmol/kg) and PVP-IR800 (3 μmol/kg). NIRF images acquired at excitation of 675 nm and emission of 720 nm. (c) R-NIRFs at tumor sites as a function of post-injection timepoints of reporters (n=3). aPD-L1 versus saline, TAMR_{M1}: P=0.0013; TAMR_{CTL}: P=0.0215. aPD-L1/oxaliplatin (Oxa) versus saline, TAMR_{M1}: P=0.0003; TAMR_{CTL}: P=0.0043; TAMR_{NE}: P=0.0048. (d) Colocalization of NIRF signals of TAMRs with their respective TILs in 4T1 tumor sections post-treatment with aPD-L1/Oxa (n=3). Venn diagram indicates the average cell numbers, and the below table shows the percentage of single and double positive cells of TAMRs and their respective TILs. (e) Representative flow cytometry plots of iNOS⁺Cas-1⁺ cells gated by F4/80⁺ cells, GrB⁺ cells gated by CD8+ cells, and Ly-6G⁺NE⁺ cells gated by CD11b⁺ cells in the tumors of mice with different treatments (n=3). (f) Correlation between biomarkers of TILs and R-NIRF of TAMRs in the tumors of 4T1 tumor-bearing mice with different treatments via a simple linear regression model. 95% confidence intervals were obtained by two-sided Student t-test analysis. (g) Schematic illustration of the dynamic landscape of TIME with different treatments, and the corresponding TILs populations as a proportion of CD45⁺ cells. Data are presented as mean±SD and analyzed by two-tailed Student's t-test.

FIG. 23 depicts real-time imaging by reference reporter PVP-IR800. (a) Real-time imaging of 4T1 tumor-bearing mice after i.v. injection of TAMRs and their reference reporter (PVP-IR800) into the mice with different treatments. 4T1 tumor-bearing mice with different treatments were intravenously injected with TAMRs (5 μmol/kg), and real-time NIRF imaging was monitored for 48 h. NIRF images of PVP-IR800 were acquired at 745 nm with excitation at 800 nm by an IVIS spectrum imaging system (n=3). (b) Quantification of NIRF intensity of PVP-IR800 at tumor sites as a function of post-injection timepoints of reporters.

FIG. 24 depicts ex vivo NIRF images (TAMR_{M1}) of 4T1 tumor-bearing mice. (a) Ex vivo NIRF images of major organs including heart, liver, spleen, lung, kidneys, and tumor of 4T1 tumor-bearing mice at 72 h post-injection timepoint of TAMR_{M1} (5 μmol/kg) and PVP-IR800 (3 μmol/kg). (b) Quantification of R-NIRF_{M1} at excised tumors. Immunotherapy versus saline groups: ***P<0.001, ****P<0.0001. (c) Quantification of TAMR_{M1} in major organs by HPLC. At 72 h post-injection of TAMR_{M1} (5 μmol/kg), the mice were dissected. Major organs were homogenized in PBS and centrifuged to remove insoluble components. The supernatant containing extracted reporters was analyzed by HPLC assay. Statistical significance was performed by two-tailed Student's t-test. ns: not significant, *P<0.05, **P<0.01, ***P<0.001, ****P<0.0001.

FIG. 25 depicts ex vivo NIRF images (TAMR_{CTL}) of 4T1 tumor-bearing mice. (a) Ex vivo NIRF images of major organs including heart, liver, spleen, lung, kidneys, and tumor of 4T1 tumor-bearing mice at 72 h post-injection timepoint of TAMR_{CTL} (5 μmol/kg) and PVP-IR800 (3 μmol/kg). (b) Quantification of R-NIRF_{CTL} at excised tumors. Immunotherapy versus saline groups: **P<0.01, ***P<0.001, ****P<0.0001. (c) Quantification of TAMR_{CTL} in major organs by HPLC. At 72 h post-injection of TAMR_{CTL} (5 μmol/kg), the mice were dissected. Major organs were homogenized in PBS and centrifuged to remove insoluble components. The supernatant containing extracted reporters was analyzed by HPLC assay. Statistical significance was performed by two-tailed Student's t-test. ns: not significant, *P<0.05, **P<0.01, ***P<0.001, ****P<0.0001.

FIG. 26 depicts ex vivo NIRF images (TAMR_{NE}) of 4T1 tumor-bearing mice. (a) Ex vivo NIRF images of major organs including heart, liver, spleen, lung, kidneys, and tumor of 4T1 tumor-bearing mice at 72 h post-injection timepoint of TAMR_{NE} (5 μmol/kg) and PVP-IR800 (3 μmol/kg). (b) Quantification of R-NIRF_{NE} at excised tumors. Immunotherapy versus saline groups: *P<0.05, ****P<0.0001. (c) Quantification of TAMR_{NE} in major organs by HPLC. At 72 h post-injection of TAMR_{NE} (5 μmol/kg), the mice were dissected. Major organs were homogenized in PBS and centrifuged to remove insoluble components. The supernatant containing extracted reporters was analyzed by HPLC assay. Statistical significance was performed by two-tailed Student's t-test. ns: not significant, *P<0.05, **P<0.01, ***P<0.001, ****P<0.0001.

FIG. 27 depicts (a) representative immunofluorescence images of 4T1 tumor sections of immunotherapeutics-treated mice after i.v. injection of TAMRM1 (5 μmol/kg) for 24 h. Images were acquired on LSM800 (Zeiss) with light sources of 405 nm laser for blue channel, 488 nm laser for green channel, and 640 nm laser for red channel. Blue fluorescence was from cell nucleus stained with 4′,6-diamidino-2-phenylindole (DAPI), green fluorescence was from macrophages stained with fluorescein isothiocyanate (FITC)-labelled F4/80 antibody, and red fluorescence was from activated TAMR_{M1}, and (b) colocalization of NIRF signals of TAMR_{M1} with F4/80⁺ cells in the 4T1 tumor sections post-treatment with Oxa and aPD-L1. Venn diagram indicates the average cell numbers, and the below table shows the percentage of single and double positive cells of TAMR_{M1} and biomarkers.

FIG. 28 depicts (a) representative immunofluorescence images of 4T1 tumor sections of immunotherapeutics-treated mice after i.v. injection of TAMR_{CTL} (5 μmol/kg) for 24 h. Images were acquired on LSM800 (Zeiss) with light sources of 405 nm laser for blue channel, 488 nm laser for green channel, and 640 nm laser for red channel, and (b) colocalization of NIRF signals of TAMR_{CTL} with CD8⁺ cells in the 4T1 tumor sections post-treatment with Oxa and aPD-L1. Venn diagram indicates the average cell numbers, and the below table shows the percentage of single and double positive cells of TAMR_{CTL} and biomarkers.

FIG. 29 depicts (a) representative immunofluorescence images of 4T1 tumor sections of immunotherapeutics-treated mice after i.v. injection of TAMR_{NE} (5 μmol/kg) for 24 h. Images were acquired on LSM800 (Zeiss) with light sources of 405 nm laser for blue channel, 488 nm laser for green channel, and 640 nm laser for red channel, and (b) colocalization of NIRF signals of TAMR_{NE} with NE⁺ cells in the 4T1 tumor sections post-treatment with Oxa and aPD-L1. Venn diagram indicates the average cell numbers, and the below table shows the percentage of single and double positive cells of TAMR_{NE} and biomarkers.

FIG. 30 depicts the flow cytometry analysis of M1 macrophages in tumor-draining lymph nodes and blood samples of 4T1 tumor-bearing mice with different treatments at day 8. (a) Gating strategies for analysis of M1 macrophages in tumor, lymph node and blood samples. (b) Representative flow cytometry plots of Cas-1⁺iNOS⁺ cells gating on iNOS⁺ cells and CD11b⁺ cells in tumor-draining lymph nodes. (c) Representative flow cytometry plots of Cas-1⁺iNOS⁺ cells gating on iNOS⁺ cells and CD11b⁺ cells in blood samples.

FIG. 31 depicts the flow cytometry analysis of CTLs in tumor-draining lymph nodes and blood samples of 4T1 tumor-bearing mice with different treatments at day 8. (a) Gating strategies for analysis of CTLs in tumor, lymph node and blood samples. (b) Representative flow cytometry plots of CD8⁺GrB⁺ cells gating on CD8⁺ cells in tumor-draining lymph nodes. (c) Representative flow cytometry plots of CD8⁺GrB⁺ cells gating on CD8⁺ cells in blood samples.

FIG. 32 depicts the flow cytometry analysis of neutrophils in tumors and blood samples of 4T1 tumor-bearing mice with different treatments at day 8. (a) Gating strategies for analysis of neutrophils in tumor and blood samples. (b) Representative flow cytometry plots of CD11b⁺Ly-6G⁺ cells gating on CD45⁺ cells in tumors. (c) Representative flow cytometry plots of CD11b⁺Ly-6G⁺ cells gating on CD45⁺ cells in blood samples.

FIG. 33 depicts the correlation between populations of TILs and R-NIRFs of TAMRs in the tumor regions of 4T1 tumor-bearing mice with different treatments. R and P values were derived using a simple linear regression model. The gray error band shows the 95% confidence intervals of the fitted line by two-sided Student t-test analysis.

FIG. 34 depicts in vivo specific real-time NIRF imaging of TILs in CT26 tumor-bearing mice. (a) Schematic illustration of immunotherapeutic process and real-time imaging of TILs. (b) Real-time imaging of CT26 tumor-bearing mice with different treatments after i.v. injection of TAMRs (5 μmol/kg) and PVP-IR800 (3 μmol/kg). NIRF images acquired at excitation of 675 nm and emission of 720 nm. (c) R-NIRFs at tumor sites as a function of post-injection timepoints of reporters (n=3). aPD-L1 versus saline, TAMR_{CTL}: P<0.0001; TAMR_{NE}: P<0.0001. aCD47 versus saline, TAMR_{M1}: P=0.0227; TAMR_{CTL}: P=0.015; TAMR_{NE}: P=0.0002. aPD-L1/aCD47 versus saline, TAMR_{M1}: P=0.0002; TAMR_{CTL}: P<0.0001; TAMR_{NE}: P<0.0001. (d) Colocalization of NIRF signals of TAMRs with their respective TILs in CT26 tumor sections post-treatment with aPD-L1/aCD47 (n=3). Venn diagram indicates the average cell numbers, and the below table shows the percentage of single and double positive cells of TAMRs and their respective TILs. (e) Representative flow cytometry plots of iNOS⁺Cas-1⁺ cells gated by F4/80⁺ cells, GrB⁺ cells gated by CD8⁺ cells, and Ly-6G⁺NE⁺ cells gated by CD11b⁺ cells in the tumors of CT26 tumor-bearing mice with different treatments (n=3). (f) Correlation between biomarkers of TILs and R-NIRFs of TAMRs in the tumors of CT26 tumor-bearing mice with different treatments via a simple linear regression model. 95% confidence intervals were obtained by two-sided Student t-test analysis. (g) Schematic illustration of the dynamic landscape of TIME with different treatments, and the corresponding TILs populations as a proportion of CD45⁺ cells. Data are presented as mean±SD and analyzed by two-tailed Student's t-test.

FIG. 35 depicts real-time imaging by reference reporter PVP-IR800. (a) Real-time imaging of CT26 tumor-bearing mice after i.v. injection of TAMRs and their reference reporter (PVP-IR800) into mice with different treatments. CT26 tumor-bearing mice with different treatments were intravenously injected with TAMRs (5 μmol/kg) and PVP-IR800 (3 μmol/kg), and real-time NIRF imaging was monitored for 48 h (n=3). NIRF images of PVP-IR800 were acquired at 745 nm with excitation at 800 nm by an IVIS spectrum imaging system. (b) Quantification of NIRF intensity of PVP-IR800 at tumor sites as a function of post-injection timepoints of reporters.

FIG. 36 depicts ex vivo NIRF images (TAMR_{M1}) of CT26 tumor-bearing mice. (a) Ex vivo NIRF images of major organs including heart, liver, spleen, lung, kidneys, and tumor of CT26 tumor-bearing mice at 72 h post-injection of TAMR_{M1} (5 μmol/kg) and PVP-IR800 (3 μmol/kg). (b) Quantification of R-NIRF_{M1} at excised tumors. ***P<0.001, ****P<0.0001. (c) Quantification of TAMR_{M1} in major organs by HPLC. At 72 h post-injection of TAMR_{M1} (5 μmol/kg), the mice were dissected. Major organs were homogenized in PBS and centrifuged to remove insoluble components. The supernatant containing extracted reporters was analyzed by HPLC assay. Statistical significance was performed by two-tailed Student's t-test. ns: not significant, *P<0.05, **P<0.01, ***P<0.001, ****P<0.0001.

FIG. 37 depicts ex vivo NIRF images (TAMR_{CTL}) of CT26 tumor-bearing mice. (a) Ex vivo NIRF images of major organs including heart, liver, spleen, lung, kidneys, and tumor of CT26 tumor-bearing mice at 72 h post-injection of TAMR_{CTL} (5 μmol/kg) and PVP-IR800 (3 μmol/kg). (b) Quantification of R-NIRF_{CTL} at excised tumors. ***P<0.001, ****P<0.0001. (c) Quantification of TAMR_{CTL} in major organs by HPLC. At 72 h post-injection of TAMR_{CTL} (5 μmol/kg), the mice were dissected. Major organs were homogenized in PBS and centrifuged to remove insoluble components. The supernatant containing extracted reporters was analyzed by HPLC assay. Statistical significance was performed by two-tailed Student's t-test. ns: not significant, *P<0.05, **P<0.01, ***P<0.001, ****P<0.0001.

FIG. 38 depicts ex vivo NIRF images (TAMR_{NE}) of CT26 tumor-bearing mice. (a) Ex vivo NIRF images of major organs including heart, liver, spleen, lung, kidneys, and tumor of CT26 tumor-bearing mice at 72 h post-injection of TAMR_{NE} (5 μmol/kg) and PVP-IR800 (3 μmol/kg). (b) Quantification of R-NIRF_{NE} at excised tumors. ***P<0.001, ****P<0.0001. (c) Quantification of TAMR_{NE} in major organs by HPLC. At 72 h post-injection of TAMR_{NE} (5 μmol/kg), the mice were dissected. Major organs were homogenized in PBS and centrifuged to remove insoluble components. The supernatant containing extracted reporters was analyzed by HPLC assay. Statistical significance was performed by two-tailed Student's t-test. ns: not significant, *P<0.05, **P<0.01, ***P<0.001, ****P<0.0001.

FIG. 39 depicts (a) representative immunofluorescence images of CT26 tumor sections of immunotherapeutics-treated mice after i.v. injection of TAMR_{M1} (5 μmol/kg) for 24 h. Images were acquired on LSM800 (Zeiss) with light sources of 405 nm laser for blue channel, 488 nm laser for green channel, and 640 nm laser for red channel. (b) Colocalization of NIRF signals of TAMR_{M1} with F4/80⁺ cells in the CT26 tumor sections post-treatment with aPD-L1 and aCD47. Venn diagram indicates the average cell numbers, and the below table shows the percentage of single and double positive cells of TAMR_{M1} and biomarkers.

FIG. 40 depicts (a) representative immunofluorescence images of CT26 tumor sections of immunotherapeutics-treated mice after i.v. injection of TAMR_{CTL} (5 μmol/kg) for 24 h. Images were acquired on LSM800 (Zeiss) with light sources of 405 nm laser for blue channel, 488 nm laser for green channel, and 640 nm laser for red channel. (b) Colocalization of NIRF signals of TAMR_{CTL} with CD8⁺ cells in the CT26 tumor sections post-treatment with aPD-L1 and aCD47. Venn diagram indicates the average cell numbers, and the below table shows the percentage of single and double positive cells of TAMR_{CTL} and biomarkers.

FIG. 41 depicts (a) representative immunofluorescence images of CT26 tumor sections of immunotherapeutics-treated mice after i.v. injection of TAMR_{NE} (5 μmol/kg) for 24 h. Images were acquired on LSM800 (Zeiss) with light sources of 405 nm laser for blue channel, 488 nm laser for green channel, and 640 nm laser for red channel. (b) Colocalization of NIRF signals of TAMR_{NE} with NE⁺ cells in the CT26 tumor sections post-treatment with aPD-L1 and aCD47. Venn diagram indicates the average cell numbers, and the below table shows the percentage of single and double positive cells of TAMR_{NE} and biomarkers.

FIG. 42 depicts the flow cytometry analysis of M1 macrophages in tumor-draining lymph nodes and blood samples of CT26 tumor-bearing mice with different treatments at day 8. (a) Gating strategies for analysis of M1 macrophages in tumor, lymph node and blood samples. (b) Representative flow cytometry plots of Cas-1⁺iNOS⁺ cells gating on iNOS⁺ cells and CD11b⁺ cells in tumor-draining lymph nodes. (c) Representative flow cytometry plots of Cas-1⁺iNOS⁺ cells gating on iNOS⁺ cells and CD11b⁺ cells in blood samples.

FIG. 43 depicts the flow cytometry analysis of CTLs in tumor-draining lymph nodes and blood samples of CT26 tumor-bearing mice with different treatments at day 8. (a) Gating strategies for analysis of CTLs in tumor, lymph node and blood samples. (b) Representative flow cytometry plots of CD8⁺GrB⁺ cells gating on CD8⁺ cells in tumor-draining lymph nodes. (c) Representative flow cytometry plots of CD8⁺GrB⁺ cells gating on CD8⁺ cells in blood samples.

FIG. 44 depicts the flow cytometry analysis of neutrophils in tumors and blood samples of CT26 tumor-bearing mice with different treatments at day 8. (a) Gating strategies for analysis of neutrophils in tumor and blood samples. (b) Representative flow cytometry plots of CD11b+Ly-6G⁺ cells gating on CD45⁺ cells in tumors. (c) Representative flow cytometry plots of CD11b+Ly-6G⁺ cells gating on CD45⁺ cells in blood samples.

FIG. 45 depicts the correlation between populations of TILs and R-NIRFs of TAMRs in the tumor regions of CT26 tumor-bearing mice with different treatments. R and P values were derived using a simple linear regression model. The error band in gray shows the 95% confidence intervals of the fitted line by two-sided Student t-test analysis.

FIG. 46 depicts the prediction of cancer immunotherapy by TAMRs. Schematic illustration of therapeutic mechanisms against (a) 4T1 tumor-bearing mice and (b) CT26 tumor-bearing mice. (c) Tumor growth curves (n=6) and (d) overall survival curves (n=6) of 4T1 tumor-bearing mice. For tumor growth curves, aPD-L1 versus saline: P=0.0005; Oxa versus saline: P<0.0001; aPD-L1/Oxa versus saline: P<0.0001. For survival curves, aPD-L1/Oxa versus saline: P=0.0008. (e) Tumor growth curves (n=6) and (f) overall survival curves (n=6) of CT26 tumor-bearing mice. For tumor growth curves, aPD-L1 versus saline: P<0.0001; aCD47 versus saline: P=0.0002; aPD-L1/aCD47 versus saline: P<0.0001. For survival curves, aPD-L1/aCD47 versus saline: P=0.0005. (g) Urinary R-NIRFs of TAMRs of tumor-bearing mice with different treatments (n=3). For 4T1 tumor-bearing mice: aPD-L1 versus saline, TAMR_{M1}: P=0.0114; TAMR_{CTL}: P=0.0123; TAMR_{NE}: ns. Oxa versus saline: TAMR_{M1}: P=0.0047; TAMR_{CTL}: ns; TAMR_{NE}: P=0.0018. aPD-L1/Oxa versus saline: TAMR_{M1}: P=0.0006; TAMR_{CTL}: P=0.0006; TAMR_{NE}: P=0.0001. For CT26 tumor-bearing mice: aPD-L1 versus saline, TAMR_{M1}: P=0.0002; TAMR_{CTL}: P=0.0186; TAMR_{NE}: P=0.0003. aCD47 versus saline: TAMR_{M1}: P<0.0001; TAMR_{CTL}: ns; TAMR_{NE}: P=0.0002. aPD-L1/aCD47 versus saline: TAMR_{M1}: P<0.0001; TAMR_{CTL}: P=0.0003; TAMR_{NE}: P<0.0001. (h) Correlation between urinary R-NIRFs of TAMRs and relative tumor volumes of tumor-bearing mice with different treatments via a simple linear regression model. 95% confidence intervals were obtained by two-sided Student t-test analysis. (i) Principal-component analysis (PCA) of R-NIRFs of TAMRs for untreated CT26 and 4T1 tumor-bearing mice. (j) Receiver-operating-characteristic (ROC) curves for TAMRs in differentiating between untreated 4T1 and CT26 tumors using R-NIRF_{M1} (area under the curve (AUC)=0.92, 95% CI=0.757-1.000), R-NIRFC_{TL} (AUC=0.98, 95% CI=0.917-1.000), R-NIRF_{NE} (AUC=0.53, 95% CI=0.182-0.874) and combination of multiple TAMRs derived from logistic regression of R-NIRFs of TAMR_{M1}, TAMR_{CTL} and TAMR_{NE} (AUC=1.00, 95% CI=1.000-1.000). (k) PCA of R-NIRFs of TAMRs for aPD-L1 treated 4T1 and CT26 tumor-bearing mice. (l) ROC curves for TAMRs in differentiating between aPD-L1 treated 4T1 and CT26 tumors using R-NIRF_{M1} (AUC=0.81, 95% CI=0.616-1.000), R-NIRF_{CTL} (AUC=0.93, 95% CI=0.781-1.000), R-NIRF_{NE} (AUC=0.96, 95% CI=0.880-1.000) and combination of multiple TAMRs derived from logistic regression of R-NIRFs of TAMR_{M1}, TAMR_{CTL} and TAMR_{NE} (AUC=1.00, 95% CI=1.000-1.000). (m) PCA of TAMRs for tumor-bearing mice with different treatments. (n) Whole-slide imaging analysis of TAMRs distribution and (o) quantification of fluorescent dots of TAMRs for tumor sections from mice with different treatments at 24 h post-injection of TAMRs (n=3). Scale bar=500 μm. Data are presented as mean±SD and analyzed by two-tailed Student's t-test.

FIG. 47 depicts the tumor control rate and body weights of mice. Tumor control rate at the end of treatments of (a) 4T1 tumor-bearing mice and (b) CT26 tumor-bearing mice. ****P<0.0001. (c) Body weights of 4T1 tumor-bearing mice after intraperitoneal injection of saline, Oxa (6 mg/kg), aPD-L1 (10 mg/kg) and aPD-L1/Oxa at day 0, 2 and 4. (d) Body weights of CT26 tumor-bearing mice after intraperitoneal injection of saline, aCD47 (6 mg/kg), aPD-L1 (10 mg/kg) and aPD-L1/aCD47 at day 0, 2 and 4. Statistical significance was performed by two-tailed Student's t-test. ns: not significant, *P<0.05, **P<0.01, ***P<0.001, ****P<0.0001.

FIG. 48 depicts the NIRF imaging of TAMRs in the urine of tumor-bearing mice with different treatments (n=3). Tumor-bearing mice treated with different treatments were intravenously injected with PVP-IR800 (3 μmol/kg) and TAMRs (5 μmol/kg) and placed into metabolic cages. At 12 post-injection timepoint, the urine samples were collected and imaged by IVIS spectrum imaging system at an exposure time of 0.1 s with excitation at 675 nm and emission at 720 nm for monitoring of activated CyOH.

FIG. 49 depicts the correlation between urinary R-NIRFs of TAMRs and in vivo R-NIRFs of TAMRs in tumor regions of tumor-bearing mice with different treatments. R and P values were derived using a simple linear regression model. The gray error band shows the 95% confidence intervals of the fitted line by two-sided Student t-test analysis.

FIG. 50 depicts the correlation between TILs and urinary R-NIRFs of TAMRs of tumor-bearing mice with different treatments. R and P values were derived using a simple linear regression model. The gray error band shows the 95% confidence intervals of the fitted line by two-sided Student t-test analysis.

FIG. 51 depicts the correlation between R-NIRFs of TAMRs in real-time imaging in the tumor regions and tumor progression presented as relative tumor volume of tumor-bearing mice with different treatments. R and P values were derived using a simple linear regression model. The gray error band shows the 95% confidence intervals of the fitted line by two-sided Student t-test analysis.

FIG. 52 depicts (a) ROC analysis showing the discrimination specificity and sensitivity between untreated and all treated 4T1 tumor-bearing mice using R-NIRFs of TAMR_{M1} (AUC=0.90, 95% CI=0.738-1.00), TAMR_{CTL} (AUC=0.89, 95% CI=0.742-1.00), TAMR_{NE} (AUC=0.84, 95% CI=0.626-1.00) and combination of multiple TAMRs (AUC=0.98, 95% CI=0.920-1.00). (b) ROC analysis showing the discrimination specificity and sensitivity between untreated and all treated CT26 tumor-bearing mice using R-NIRFs of TAMR_{M1} (AUC=0.83, 95% CI=0.683-0.971), TAMR_{CTL} (AUC=0.88, 95% CI=0.731-1.00), TAMR_{NE} (AUC=0.97, 95% CI=0.914-1.00) and combination of multiple TAMRs (AUC=1.00, 95% CI=1.00-1.00). The dashed line indicates a random diagnostic tool with an AUC of 0.5.

FIG. 53 depicts fluorescence intensity distribution analysis of TAMRs in the whole-mount scanning of tumor sections as a function of radius from the center of the slices. The quantification data was obtained by Image J analysis.

### DESCRIPTION

In a first aspect of the invention, there is provided a compound of formula I:

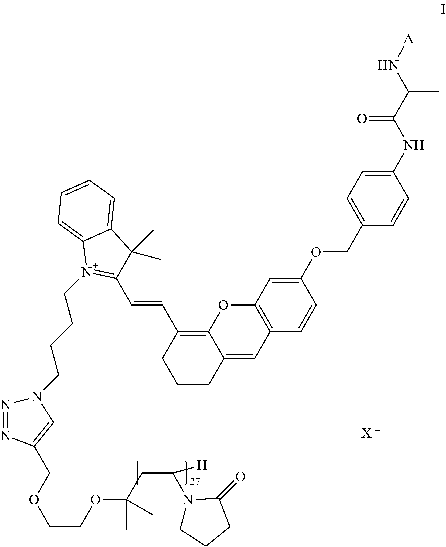

wherein:
X⁻ represents a counterion; and
A represents an amino acid moiety that is cleavable by an enzyme associated with a leukocyte, or a pharmaceutically acceptable salt or solvate thereof.
X⁻ may be any suitable counterion, such as a halogen counterion (e.g. Cl⁻, Br⁻, or I⁻).

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, and the like.

References herein (in any aspect or embodiment of the invention) to compounds of formula I include references to such compounds perse, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively, the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

In embodiments of the first aspect, A may be selected from:

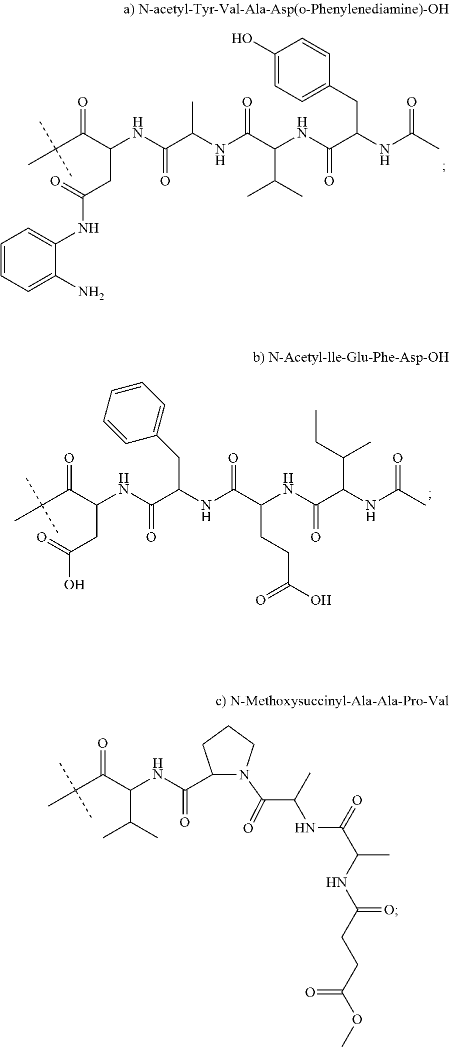

where the point of attachment is denoted by the dotted line.

The reference for (a): J. Leukoc. Biol. 2016, 100, 961; Nat. Med. 2016, 22, 64; J. Biol. Chem. 1997, 272, 9677; and Adv. Mater. 2020, 32, 2000648. The reference for (b): J. Biol. Chem. 2007, 282, 4545; and Cancer Res. 2017, 77, 2318. The reference for (c): J. Biol. Chem. 1979, 254, 4027.

The compounds of formula I may be used to help determine whether a subject is suffering from a particular disease. Thus, in further aspects of the invention, there is provided:
(aa) use of a compound of formula I as defined herein, or a salt or solvate thereof in the preparation of an imaging agent for the diagnosis of a condition or disease in a tissue and/or an organ using near-infrared fluorescence;
(ab) a compound of formula I as defined herein, or a salt or solvate thereof for use as an imaging agent for the diagnosis of a condition or disease in a tissue and/or an organ using near-infrared fluorescence; and
(ac) a method of diagnosing a condition or disease in a tissue and/or an organ, the method comprising the steps of administering a compound of formula I as defined herein, or a salt or solvate thereof to a subject in need of diagnosis and determining the presence or absence of a condition or disease in a tissue and/or an organ using near-infrared fluorescence.

The use of (aa) or the compound for use of (ab) may be conducted in vivo or in vitro. In particular embodiments, the in vivo imaging may be for the purpose of visualizing the tumour immune microenvironment.

The compounds of formula I may also be used to determine whether a particular tumor tissue is susceptible to immunotherapy. Thus, in a further aspect of the invention, there is provided a method of determining the susceptibility of a tumour tissue to an immunotherapy, the method comprising the steps of:
(i) subjecting a tumour tissue to a desired course of immunotherapy;
(ii) administering a compound of formula I as defined herein, or a salt or solvate thereof to the tumour tissue along with an always on reference reporter compound;
(iii) after a period of time determining the ratio of near-infrared fluorescence obtained from a metabolite of the compound of formula I as defined herein relative to the always on reference reporter compound; and
(iv) determining the susceptibility of the tumour tissue to the immunotherapy based on the ratio obtained in step (iii).

This method may be conducted in vivo or in vitro.

The method above, whether conducted in vitro or in vitro enables the determination of whether a particular tissue can be treated by immunotherapy. Given this, the skilled person can then treat the tumour if it is revealed to be susceptible to the proposed treatment. Thus, in certain embodiments, the method may further involve subjecting a subject to a therapeutic treatment regimen based on the results obtained by the method.

Any suitable always on reference reporter compound may be used herein. For example, the reference reported compound may be:

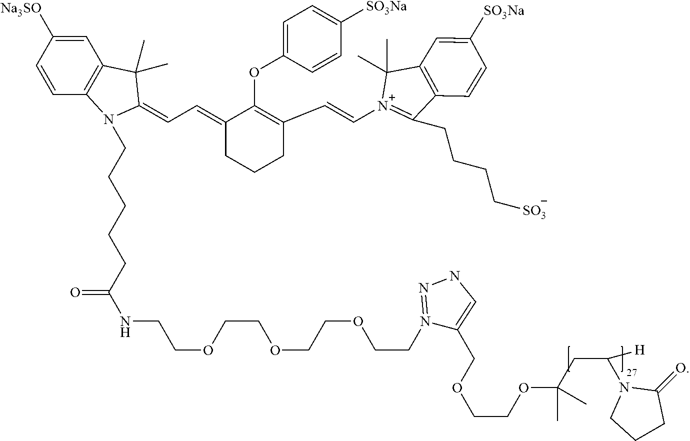

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

The compounds of formula I may be prepared for administration to a subject. Thus, in a further aspect of the invention, there is provided a composition comprising a compound of formula I, or a pharmaceutically acceptable salt or solvate thereof as described herein in admixture with one or more of a pharmaceutically acceptable adjuvant, diluent and carrier.

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic or diagnostic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

Aspects and embodiments of the invention will now be discussed by reference to the following non-limiting examples.

### EXAMPLES

### Materials

All chemicals were purchased from Sigma-Aldrich unless otherwise stated. All amino acid derivatives were bought from GL Biochem. PBr₃ and p-aminobenzyl alcohol were purchased from Tokyo Chemical Industry. Anti-mouse PD-L1 (B7-H1) (Clone: 10F.9G2, Cat No. BP0101) was purchased from Bio X Cell. Anti-mouse CD16/32 (Cat No. 101302), Alexa Fluor® 700 anti-mouse CD45 (Cat No. 103128), FITC anti-mouse CD3 (Cat No. 100204), PE anti-mouse CD8a (Cat No. 100708), APC anti-human/mouse GrB recombinant antibody (Cat No. 372204), PerCP anti-mouse/human CD11b (Cat No. 101229), ultra-LEAF™ Purified anti-mouse CD47 antibody (Cat No. 127518), recombinant mouse murine granulocyte macrophage colony stimulating factor (GM-CSF, Cat No. 576304), recombinant rat interferon-gamma (IFN-γ, Cat No. 598802), recombinant rat interleukin-4 (IL-4, Cat No. 776902), intracellular staining perm wash buffer (ISPWB), ACK lysis, PE anti-mouse/human CD11b, and APC anti-mouse Ly-6G were purchased from Biolegend. Neutrophil elastase (NE) polyclonal antibody (PA5-79198), anti-Mo Ly-6G APC (17-9668-82), anti-Mouse NOS2 PE (12-5920-82), Live/Dead™ Fixable Blue Dead Cell Stain (Cat No. L23105), Dynabeads™ FlowComp™ Mouse CD8 kit (Cat No. 11462D) and secondary antibody Alexa Fluor® 488 conjugated goat anti-rabbit IgG (Cat No. 2045215) were purchased from Thermo Fisher Scientific. Caspase-1 (D-3) Alexa Fluor® 647 and F4/80 (C-7) Alexa Fluor® 488 (sc-377009) were purchased from Santa Cruz Biotechnology. GrB was purchased from Novoprotein. Caspase-1 was purchased from BioVision. Cathepsin C (DPPI) and APN were obtained from R&D systems. NE, LPS, bovine serum albumin (BSA), HEPES, CHAPS (3-[(3-cholamidopropyl)dimethylammonio]propanesulfonate), PBS, heat-inactivated horse serum, interleukin-2 (IL-2) and DNase I were purchased from Sigma-Aldrich. MTS solution was purchased from PROMEGA PTE LTD. Dulbecco's modified eagle medium (DMEM), fetal bovine serum (FBS), Iscove's Modified Dulbecco's Medium (IMDM) and Roswell Park Memorial Institute (RPMI) were purchased from GIBCO. O.C.T. medium was bought from Sakura Fineteck Japan. Heparinized capillary tubes were purchased from Greiner Bio-One GmbH. Type I collagenase and type IV collagenase were purchased from Thermofisher. Dialysis bag was used for dialysis. Dialysis bags were bought from USA Viskase.

Mouse breast cancer cell line 4T1 cells, murine colorectal carcinoma cell line CT26 cells, mouse embryonic fibroblasts 3T3 cells, mouse neutrophils MPRO cells, and mouse macrophage cell line RAW 264.7 cells were purchased from the American Type Culture Collection (ATCC).

### Analytical Techniques

### UV-Vis Spectroscopy

UV-Vis spectra were recorded on a Shimadzu UV-2450 spectrophotometer.

### Fluorescence Spectroscopy

Fluorescence spectra were recorded on a Fluorolog 3-TCSPC spectrofluorometer (Horiba Jobin Yvon).

### HPLC

HPLC curves were measured on an Agilent 1260 system equipped with UV detector, G1311B pump, and an Agilent Zorbax SB-C18 RP (9.4×250 mm) column, with CH₃OH containing trifluoroacetic acid (TFA, 0.1%) and water containing TFA (0.1%) as the eluent.

TABLE 1
Enzyme kinetics parameters of TASMRs and CyA.

| | | |
|---|---|---|
| Km | Vmax | Kcat/Km |

| | | | | | | |
|---|---|---|---|---|---|---|
| Cy-DAVY | 36.6 | μM | 0.015 | nmol/min | 0.003 | μM⁻¹s⁻¹ |
| TASMR_{CTL} | 37.3 | μM | 0.045 | nmol/min | 0.002 | μM⁻¹s⁻¹ |
| TASMR_{NE} | 40.3 | μM | 13.3 | pmol/s | 0.49 | μM⁻¹s⁻¹ |
| CyA | 22.0 | μM | 1.6 | pmol/s | 0.03 | μM⁻¹s⁻¹ |

TABLE 2
HPLC condition for the enzymatic analysis.

| Time (min) | Flow (mL/min) | H₂O % | CH₃OH % |
|---|---|---|---|
| 0 | 3 | 70 | 30 |
| 3 | 3 | 70 | 30 |
| 35 | 3 | 10 | 90 |
| 37 | 3 | 10 | 90 |
| 38 | 3 | 70 | 30 |
| 40 | 3 | 70 | 30 |

### Proton-Nuclear Magnetic Resonance (¹H NMR) Spectroscopy

¹H NMR spectra were recorded on a Bruker 400 MHz NMR.

### Liquid Chromatography-Mass Spectrometry (LCMS)

LCMS spectra were measured on Thermo Finnigan Polaris Q quadrupole ion trap mass spectrometer equipped with a standard electrospray ionization (ESI) source.

### Fluorescence Imaging

Fluorescence imaging of cells was acquired on Laser Scanning Microscope LSM800 (Zeiss).

### In Vivo Animal Fluorescence Imaging

In vivo animal fluorescence images were taken via an IVIS imaging system (IVIS-CT machine, PerkinElmer), and the region of interest was analyzed via the Living Image 4.3 software.

### Tissue Sections

Tissue sections were obtained on a cryostat (Leica).

### General Procedure for High Performance Liquid Chromatography (HPLC) Purification

HPLC purification was performed on an Agilent 1260 gradient preparative system equipped with a G1361A pump, UV detector and an Agilent Zorbax SB-C18 RP (21.2×150 mm) column, with CH₃OH containing TFA (0.1%) and water containing TFA (0.1%) as the eluent (Tables 2 and 3).

TABLE 3
HPLC condition for the purification
of TASMRs and their precursors.

| Time (min) | Flow (mL/min) | H₂O % | CH₃OH % |
|---|---|---|---|
| 0 | 12 | 70 | 30 |
| 3 | 12 | 70 | 30 |
| 35 | 12 | 10 | 90 |
| 37 | 12 | 10 | 90 |
| 38 | 12 | 70 | 30 |
| 40 | 12 | 70 | 30 |

### General Procedure for Cell Culture

All cells were cultured in a humidified environment at 37° C. which contains 5% CO₂ and 95% air. 3T3 and RAW 264.7 cells were cultured in DMEM with 10% FBS. 4T1 and CT26 cells were cultured in RPMI 1640 with 10% FBS. MPRO cells were cultured in IMDM with 4 mM L-glutamine adjusted to contain 1.5 g/L sodium bicarbonate containing 10 ng/mL murine GM-CSF, and 20% of heat-inactivated horse serum. CD8 T cells were isolated from spleen of BALB/c mouse by using the Dynabeads® Untouched™ Mouse CD8 Cells kit, and cultured in RPMI 1640 with IL-2. Bone marrow-derived dendritic cells (BMDCs) were isolated from bone marrow of BALB/c mice according to previous protocols (Madaan, A. et al., J. Biol. Methods 2014, 1, e1) and cultured in RPMI 1640 with GM-CSF (20 ng/mL). RAW264.7 cells were polarized to M1 macrophages by incubation with LPS (10 μg/mL) and IFN-γ (20 ng/mL) for 24 h. RAW264.7 cells were polarized to M2 macrophages by incubation with IL-4 (20 ng/mL) for 24 h. Both M1 and M2 macrophages were cultured in DMEM.

### General Procedure for Establishment of Tumor-Bearing Mice

All mouse experimental procedures were approved by the Institutional Animal Care and Use Committee (IACUC), Nanyang Technological University (NTU). Six-week-old female BALB/c mice were purchased from InVivos, Singapore. For establishment of poorly immunogenic tumors, 4T1 cancer cells in PBS were subcutaneously implanted to the right flank of mice at a density of 1×10⁶ cells/mouse. For establishment of highly immunogenic tumors, CT26 cancer cells in PBS were subcutaneously implanted to the right side of the back of mice at a density of 2×10⁶ cells/mouse.

### Statistics and Reproducibility

The in vivo and ex vivo fluorescence intensities were quantified using Living Image 4.3 software for the region of interest analysis. Statistical comparisons between two groups were determined by two-tailed Student's t-test. For statistics analysis, P<0.05 was considered statistically significant; *P<0.05, **P<0.01, ***P<0.001, and ****P<0.0001.

### Example 1. Synthesis of TAMRs

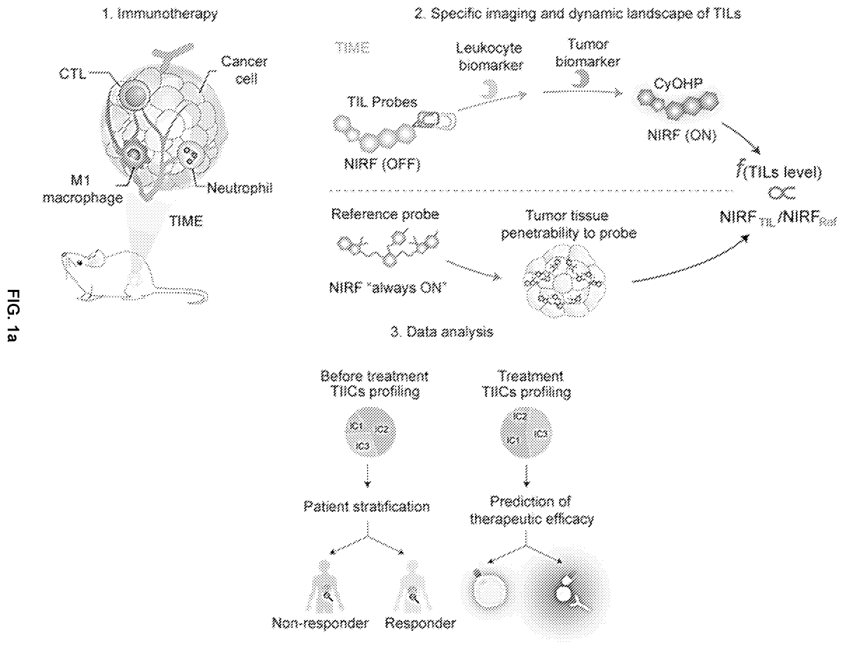

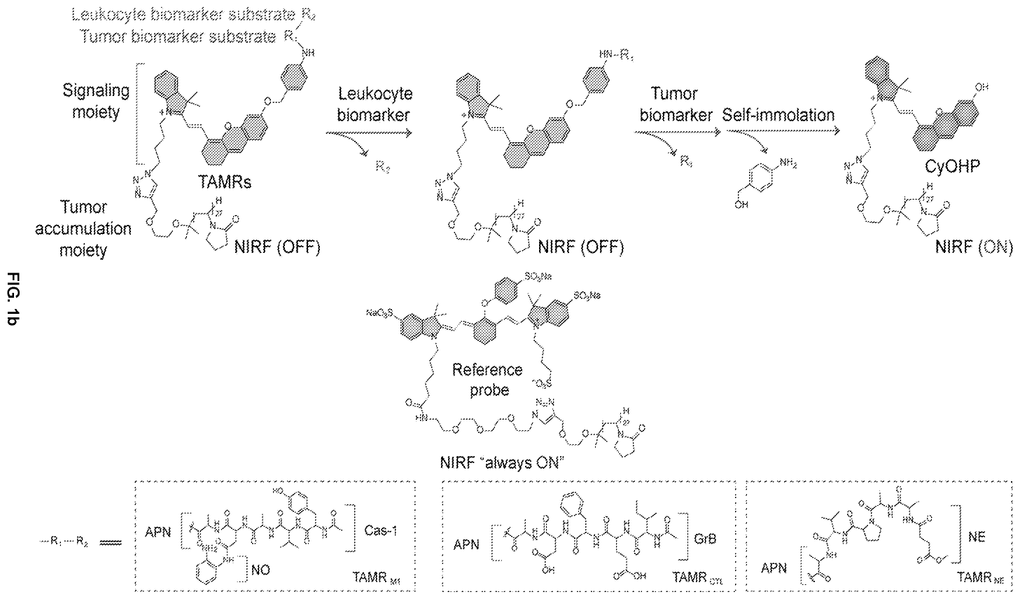

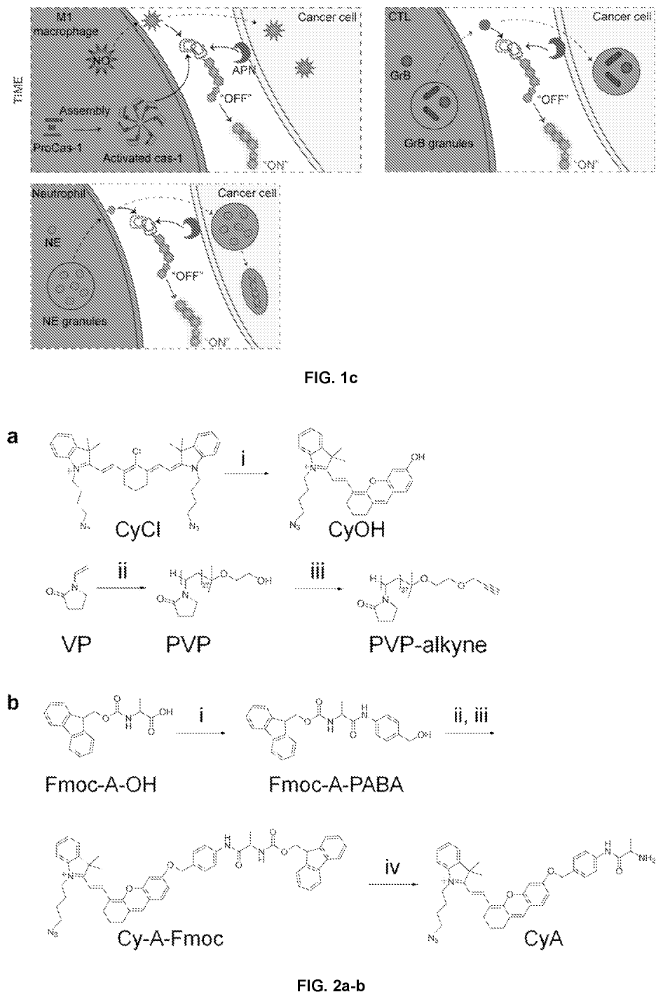

TAMRs comprise three key units: a tumor-passive targeting moiety, a fluorescent signaling moiety, and a dual-lock TILs responsive moiety that can only be fully cleaved in the presence of both cancer and leukocytes (FIG. 1). TAMRs are intrinsically non-fluorescent, and only activate their NIRF signals in the presence of both cancer and leukocyte biomarkers. Such a dual-locked tandem design ensures the signal activation triggered only by TILs but not by leukocytes in other normal and inflammatory tissues. TAMR_{M1}, TAMR_{CTL}, and TAMR_{NE} were synthesized to respectively detect M1 macrophages, CTLs and neutrophils, permitting real-time dynamic imaging of TIME. To rule out the accumulation differences of TAMRs in tumors, PVP-IR800 was synthesized as an “always-on” reference probe and used for normalization in the process of signal analysis.

### Synthesis of PVP-IR800

IR800-N₃ (5 mg, 0.004 mmol) and PVP-alkyne (12 mg, 0.004 mmol) were dissolved in DMSO (1.5 mL). Then, CuSO₄·5H₂O (1 mg, 0.004 mmol) and sodium ascorbate (1.6 mg, 0.008 mmol) in deionized water (1.5 mL) were added. The reaction mixture was stirred at 25° C. for 12 h. Then, the reaction solution was dialyzed against deionized water for 24 h to remove salts and DMSO, and freeze-dried to get PVP-IR800.

TAMRs were constructed on a near-infrared (NIR) hemicyanine dye (CyOH) (FIG. 2) through the following three steps. First, the peptide substrate cleavable by the cancer biomarker (APN) alanine (A) was conjugated to CyOH to yield CyA. Then, the leukocyte-biomarker-reactive peptide substrates including Cas-1 cleavable peptide substrate N-acetyl-Tyr-Val-Ala-Asp-OH (YVAD) for M1 macrophages, GrB cleavable peptide substrate N-acetyl-Ile-Glu-Phe-Asp-OH (IEFD) for CTLs, and NE cleavable peptide substrate N-methoxysuccinyl-Ala-Ala-Pro-Val (AAPV) for neutrophils were respectively conjugated to CyA to afford the TAMR precursors TASMR_{M1}, TASMR_{CTL}, and TASMR_{NE}. For TASMR_{M1}, NO cleavable substrate, OPD, was conjugated to the carboxyl (COOH) side chain group of YVAD to enhance the specificity towards M1 macrophages. At last, the azide group of the TAMR precursors was clicked with alkyne-functionalized PVP to afford TAMRs. Therefore, the tandem design is modular and can be generalized for different TILs as shown by the syntheses of TAMR_{M1}, TAMR_{CTL} and TAMR_{NE} detecting tumor-infiltrating M1 macrophages, CTLs and neutrophils, respectively (FIG. 2).

### Synthesis of CyOH

CyCl was synthesized according to the previous protocol (Huang, J. et al., Nat. Mater. 2019, 18, 1133-1143). K₂CO₃ (1382 mg, 10 mmol) and resorcinol (1101 mg, 10 mmol) were dissolved in CH₃CN (10 mL), followed by stirring at 55° C. for 20 min. Then, a CH₃CN solution of CyCl (16.25 mg, 5 mmol) was added to the reaction mixture. The reaction mixture was stirred for another 6 h at 55° C., followed by removing CH₃CN. The crude product was purified by silica gel column chromatography using DCM and methanol (CH₃OH) (DCM/CH₃OH=30/1) to obtain CyOH with a yield of 79%.

MS of CyOH: m/z 467.36. ¹H NMR (400 MHz, MeOD): δ (ppm): 8.38 (d, J=12.0 Hz, 1H), 7.59 (s, 1H), 7.49 (d, J=8, 1H), 7.38 (t, 2H), 7.21 (t, 2H), 6.73 (d, J=8, 1H), 6.57 (s, 1H), 6.05 (d, J=16, 1H), 4.07 (t, 2H), 3.45 (t, 2H), 2.74 (t, 2H), 2.68 (t, 2H), 1.91-1.89 (m, 4H), 1.79-1.74 (m, 2H), 1.74 (s, 6H).

### Synthesis of PVP

N-vinyl pyrrolidinone (1 g, 9 mmol) and AIBN (20 mg) were dissolved in isopropoxyethanol (10 mL), followed by flushing with nitrogen for 10 min. The reaction mixture was stirred at 60° C. for 4 h under the protection of nitrogen. After completion, isopropoxyethanol was concentrated, and the residue in isopropoxyethanol was precipitated into an excess of ethyl ether (200 mL). The white precipitate was centrifuged at 7500 rpm for 10 min and dried at 37° C. under vacuum to get PVP with a yield of 67%.

¹H NMR (400 MHz, MeOD): δ (ppm): 3.92-3.74 (m, 32H), 3.29 (m, 54H), 2.38-2.27 (m, 54H), 2.06-2.01 (m, 54H), 1.75-1.46 (m, 60H).

### Synthesis of PVP-Alkyne

PVP (3 g, 1 mmol) was dissolved in anhydrous THF (40 mL), and NaH (240 mg, 10 mmol) was added. After production of bubbles for 10 min, 3-bromopropyne (1.19 g, 10 mmol) was quickly added to the reaction solution, followed by stirring at 25° C. for 24 h. After completion, THF was concentrated, and the mixture in THF was precipitated into an excess of ethyl ether (200 mL). The white precipitate was centrifuged at 7500 rpm for 10 min. The residue was further dissolved in water and dialyzed against deionized water to remove salts for 4 h (MWCO=1000) and freeze-dried to get PVP-alkyne with a yield of 77%.

¹H NMR (400 MHz, MeOD): δ (ppm): 4.17 (s, 2H), 3.92-3.74 (m, 32H), 3.29 (m, 54H), 2.51 (s, 2H), 2.38-2.27 (m, 54H), 2.06-2.01 (m, 54H), 1.75-1.46 (m, 60H).

### Synthesis of Ac-Y(tBu)VAD(OtBu)—OH, Ac—IE(OtBu)FD(OtBu)—OH, and MeOSuc-AAPV-OH

Peptides Ac-Y(tBu)VAD(OtBu)—OH, Ac—IE(OtBu)FD(OtBu)—OH, and MeOSuc-AAPV-OH were prepared by solid phase peptide synthesis (SPPS, O. A. Musaimi, B. G. de la Torre & F. Albericio, Green Chem. 2020, 22, 996-1018).

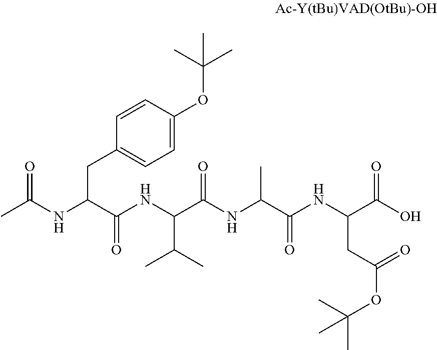 [SMILES: CC(=O)NC(Cc1ccc(OC(C)(C)C)cc1)C(=O)NC(C(=O)NC(C)C(=O)NC(CC(=O)OC(C)(C)C)C(=O)O)C(C)C]

¹H NMR (400 MHz, MeOD): δ (ppm): 7.18 (d, J=8, 2H), 6.92 (d, J=8, 2H), 4.75 (t, 1H), 4.67-4.63 (m, 1H), 4.44-4.39 (m, 1H), 4.23-4.19 (m, 1H), 3.14-3.09 (m, 1H), 2.88-2.82 (m, 2H), 2.77 (d, J=4, 2H), 1.92 (s, 3H), 1.46 (s, 9H), 1.40 (d, J=8, 3H), 1.33 (s, 9H), 0.98 (t, 6H).

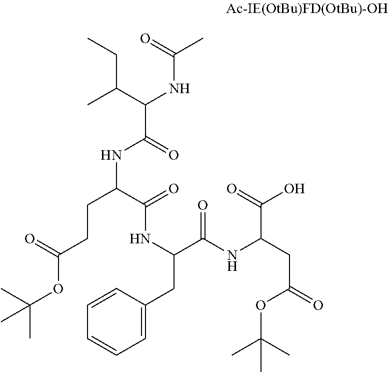 [SMILES: CCC(C)C(NC(C)=O)C(=O)NC(CCC(=O)OC(C)(C)C)C(=O)NC(Cc1ccccc1)C(=O)NC(CC(=O)OC(C)(C)C)C(=O)O]

¹H NMR (400 MHz, MeOD): δ (ppm): 7.26-7.15 (m, 5H), 4.76 (t, 1H), 4.67-4.63 (m, 1H), 4.32-4.28 (m, 1H), 4.15 (d, J=8, 1H), 3.25 (m, 2H), 3.2 (m, 1H), 2.98-2.92 (m, 1H), 2.79-2.70 (m, 2H), 2.30-2.09 (m, 2H), 2.01 (s, 3H), 1.97-1.90 (m, 1H), 1.86-1.75 (m, 2H), 1.45 (s, 18H), 0.93-0.83 (m, 6H).

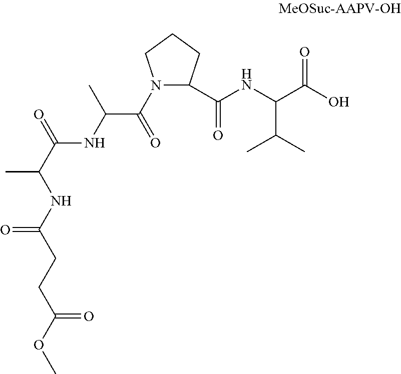 [SMILES: COC(=O)CCC(=O)NC(C)C(=O)NC(C)C(=O)N1CCCC1C(=O)NC(C(=O)O)C(C)C]

¹H NMR (400 MHz, MeOD): δ (ppm): 4.64-4.52 (m, 2H), 4.35 (t, 1H), 4.29 (t, 1H), 3.81-3.75 (m, 1H), 3.66 (s, 3H), 3.32-3.30 (m, 1H), 2.69-2.58 (m, 2H), 2.55-2.45 (m, 2H), 2.21-2.18 (m, 2H), 2.04-1.91 (m, 3H), 1.36-1.32 (m, 6H), 0.99 (d, J=8, 6H).

### Synthesis of Fmoc-A-PABA

EEDQ (742 mg, 3.0 mmol), PABA (369 mg, 3.0 mmol) and Fmoc-A-OH (311.3 mg, 1.0 mmol) were dissolved in DCM (15 mL), and the reaction mixture was continuously stirred at 25° C. for 6 h. The residues were purified by HPLC and freeze-dried to get Fmoc-A-PABA with a yield of 91%.

¹H NMR (400 MHz, MeOD): δ (ppm): 7.82 (d, J=8, 2H), 7.70 (t, 2H), 7.56 (d, J=8, 2H), 7.40 (t, 2H), 7.33 (d, J=8, 4H), 4.58 (s, 2H), 4.41 (d, J=8, 2H), 4.29-4.22 (m, 2H), 1.44 (d, J=8, 3H).

### Synthesis of Cy-A-Fmoc

Fmoc-A-PABA (100 mg, 0.24 mmol) was dissolved in anhydrous THF, followed by adding PBr₃ (200 mg, 0.72 mmol). The reaction mixture was stirred at 0° C. for 2 h. Then, THF was removed and the residue was dissolved with an excess amount of ethyl acetate (EA, 200 mL). The solution was washed with NaHCO₃ aqueous solution for three times. After concentrated and dried, the residue was dissolved by anhydrous CH₃CN (50 mL), followed by adding CyOH (37.4 mg, 0.08 mmol) and N,N-diisopropylethylamine (DIPEA, 40 μL). The reaction mixture was stirred at 55° C. for 8 h. Then, CH₃CN was removed, and the residue was purified by HPLC and freeze-dried to get Cy-A-Fmoc with a yield of 97%.

¹H NMR (400 MHz, CDCl₃): δ (ppm): 8.66 (d, J=12, 1H), 7.75 (d, J=8, 2H), 7.67 (d, J=8, 3H), 7.60 (t, 2H), 7.46-7.40 (m, 6H), 7.32 (t, 3H), 7.24 (t, 2H), 7.01-6.98 (m, 2H), 6.44 (d, J=16, 1H), 5.22 (s, 2H), 4.31-4.23 (m, 5H), 4.10 (t, 1H), 3.40 (t, 2H), 2.71 (t, 2H), 2.65 (t, 2H), 1.89-1.86 (m, 4H), 1.75 (s, 6H), 1.74-1.7 (m, 2H), 1.41 (d, J=8, 3H).

### Synthesis of CyA

Cy-A-Fmoc (10 mg, 0.012 mmol) was dissolved in DMF (2 mL). Piperidine (100 μL) was added to the solution, followed by stirring at 25° C. for 5 min. The mixture was purified by HPLC and freeze-dried to get CyA with a yield of 88%.

MS of CyA: m/z 643.43. ¹H NMR (400 MHz, CDCl₃): δ (ppm): 8.82 (d, J=16, 1H), 7.91 (d, J=8, 2H), 7.75 (d, J=8, 2H), 7.71-7.68 (m, 2H), 7.57 (d, J=4, 1H), 7.53-7.48 (m, 3H), 7.15 (d, J=4, 1H), 7.09-7.05 (m, 1H), 6.56 (d, J=12, 1H), 5.27 (s, 2H), 4.51 (t, 1H), 4.40 (t, 1H), 4.12-4.07 (m, 1H), 3.17 (t, 2H), 3.0 (t, 2H), 2.81 (t, 1H), 2.75 (t, 1H), 1.86 (s, 6H), 1.83-1.77 (m, 6H), 1.63 (d, J=8, 3H).

### Synthesis of CyA-D(OtBu)AVY(tBu)

CyA (10 mg, 0.016 mmol), Ac-Y(tBu)VAD(OtBu)—OH (29.8 mg, 0.036 mmol), HBTU (12.1 mg, 0.032 mmol), HOBt (4.3 mmg, 0.032 mmol), and DIPEA (4.1 mg, 0.032 mmol) were dissolved in DMF (5 mL), followed by stirring at 25° C. for 2 h. Then, the reaction mixture was purified by HPLC and freeze-dried to get CyA-D(OtBu)AVY(tBu) with a yield of 91%.

¹H NMR (400 MHz, CDCl₃): δ (ppm): 8.79 (d, J=16, 1H), 8.21 (t, 1H), 8.15 (d, J=8, 1H), 8.00 (d, J=12, 3H), 7.74-7.72 (m, 2H), 7.63-7.57 (m, 2H), 7.50-7.43 (m, 3H), 7.17 (d, J=8, 1H), 7.06-6.98 (m, 3H), 6.55 (d, J=16, 1H), 5.27 (s, 2H), 4.67-4.60 (m, 1H), 4.47-4.37 (m, 2H), 4.31-4.24 (m, 2H), 4.11-4.08 (m, 2H), 3.46 (t, 2H), 3.19 (s, 2H), 3.09-3.05 (m, 1H), 2.81-2.70 (m, 6H), 2.21 (t, 1H), 2.11-2.03 (m, 2H), 2.01-1.97 (m, 2H), 1.93 (s, 3H), 1.83 (s, 6H), 1.80-1.77 (m, 1H), 1.48 (d, J=8, 3H), 1.39 (s, 9H), 1.35 (d, J=8, 3H), 1.31 (s, 9H), 1.02-0.9 (m, 6H).

### Synthesis of CyA-DAVY

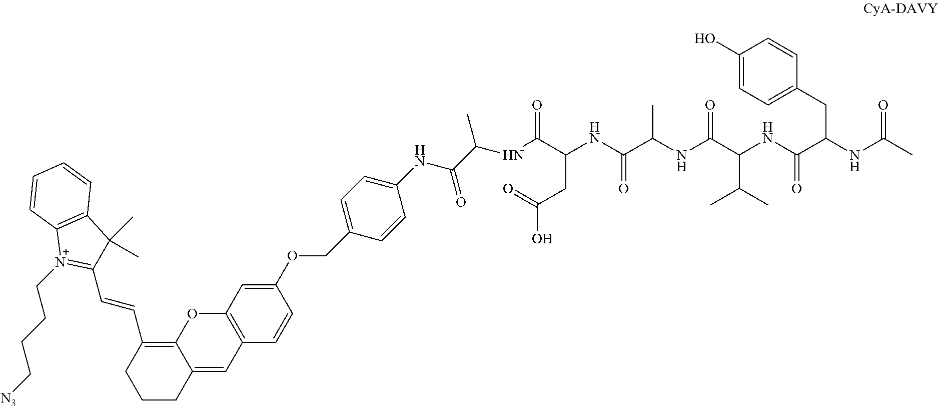 [SMILES: CC(=O)NC(Cc1ccc(O)cc1)C(=O)NC(C(=O)NC(C)C(=O)NC(CC(=O)O)C(=O)NC(C)C(=O)Nc1ccc(COc2ccc3c(c2)OC2=C(/C=C/C4=[N+](CCCCN=[N+]=[N-])c5ccccc5C4(C)C)CCCC2=C3)cc1)C(C)C]

CyA-D(OtBu)AVY(tBu) (8 mg) was dissolved in TFA (0.95 mL), followed by adding H₂O (0.05 mL). The reaction was stirred at 0° C. and monitored by HPLC. After confirming the whole deprotection of tBu and OtBu group, saturated NaHCO₃ aqueous solution was added dropwise to neutralize TFA. Then, the mixture was extracted by DCM. After drying with anhydrous Na₂SO₄, DCM was removed, and the residue was by purified by HPLC and freeze-dried to get the product CyA-DAVY with a yield of 52%.

MS of CyA-DAVY: m/z 1133.56. ¹H NMR (400 MHz, CDCl₃): δ (ppm): 8.78 (d, J=12, 1H), 8.21 (t, 1H), 7.95-7.91 (m, 1H), 7.76-7.71 (m, 3H), 7.58-7.55 (m, 2H), 7.50-7.43 (m, 4H), 7.39-7.33 (m, 2H), 7.05 (t, 2H), 6.70 (d, J=8, 1H), 6.54 (d, J=12, 1H), 5.27 (s, 2H), 4.65-4.62 (m, 1H), 4.59-4.55 (m, 1H), 4.47-4.44 (m, 1H), 4.39 (t, 2H), 4.26 (t, 1H), 4.10 (t, 1H), 3.26-3.15 (m, 2H), 3.04-2.99 (m, 1H), 2.94-2.92 (m, 1H), 2.91-2.89 (m, 1H), 2.87 (t, 2H), 2.83-2.77 (m, 2H), 2.76-2.72 (m, 2H), 2.23-2.18 (m, 1H), 2.08-2.05 (m, 2H), 2.0-1.97 (m, 2H), 1.93 (s, 3H), 1.83 (s, 6H), 1.71 (m, 1H), 1.48 (d, J=8, 3H), 1.35 (d, J=8, 3H), 1.02-0.9 (m, 6H).

### Synthesis of TASMR_{M1}

CyA-DAVY (10 mg, 0.009 mmol), OPD (4.9 mg, 0.09 mmol), HBTU (6.8 mg, 0.018 mmol), HOBt (2.4 mg, 0.018 mmol), and DIPEA (2.3 mg, 0.018 mmol) were dissolved in DMF (3 mL). The reaction mixture was stirred at 25° C. for 0.5 h. Then, the reaction mixture was purified by HPLC and freeze-dried to get TASMR_{M1} with a yield of 41%.

MS of TASMR_{M1}: m/z 612.89. ¹H NMR (400 MHz, CDCl₃): δ (ppm): 8.76 (d, J=12, 1H), 8.00 (m, 3H), 7.78-7.72 (m, 3H), 7.64-7.56 (m, 3H), 7.50-7.42 (m, 5H), 7.09-6.98 (m, 4H), 6.70 (d, J=16, 2H), 6.55 (d, J=16, 1H), 5.28 (s, 2H), 4.59-4.55 (m, 1H), 4.41-4.35 (m, 3H), 4.33-4.24 (m, 2H), 4.11 (t, 1H), 3.46 (t, 2H), 3.15-3.08 (m, 2H), 2.95-2.91 (m, 1H), 2.83-2.78 (m, 2H), 2.76-2.72 (m, 3H), 2.65-2.59 (m, 1H), 2.23-2.18 (m, 2H), 2.08-2.03 (m, 4H), 1.93 (s, 3H), 1.83 (s, 6H), 1.46 (d, J=8, 3H), 1.37 (d, J=8, 3H), 1.02-0.9 (m, 6H).

### Synthesis of CyA-D(OtBu)FE(OtBu)I

CyA (10 mg, 0.016 mmol), Ac-IE(OtBu)FD(OtBu)—OH (21.7 mg, 0.032 mmol), HBTU (12.1 mg, 0.032 mmol), HOBt (4.3 mmg, 0.032 mmol), and DIPEA (4.1 mg, 0.032 mmol) were dissolved in DMF (5 mL). The reaction mixture was stirred at 25° C. for 2 h. Then, the reaction mixture was purified by HPLC and freeze-dried to get CyA-D(OtBu)FE(OtBu)I with a yield of 91%.

¹H NMR (400 MHz, CDCl₃): δ (ppm): 8.78 (d, J=16, 1H), 8.36-8.0 (m, 2H), 7.75 (t, 2H), 7.57-7.55 (m, 2H), 7.47 (t, 3H), 7.42 (s, 1H), 7.28-7.22 (m, 5H), 7.08-7.05 (m, 2H), 6.54 (d, J=12, 1H), 5.28 (s, 2H), 4.65 (t, 1H), 4.56-4.53 (m, 1H), 4.43-4.37 (m, 3H), 4.23-4.20 (m, 1H), 4.14-4.11 (m, 1H), 3.47 (t, 2H), 3.25-3.20 (m, 2H), 3.06-3.0 (m, 1H), 2.83-2.72 (m, 7H), 2.25-2.14 (m, 2H), 2.07 (s, 3H), 1.97-1.93 (m, 5H), 1.84-1.76 (m, 8H), 1.48 (s, 3H), 1.41 (s, 18H), 1.3-1.19 (m, 2H), 0.96-0.94 (m, 6H).

### Synthesis of TASMR_{CTL}

CyA-D(OtBu)FE(OtBu)I (6.5 mg) was dissolved in TFA (0.95 mL), followed by adding H₂O (0.05 mL). The reaction was stirred at 0° C. and monitored by HPLC. After confirming the whole deprotection of OtBu group, saturated NaHCO₃ aqueous solution was added dropwise until neutralizing trifluoroacetic acid. Then, the mixture was extracted by DCM. After drying with anhydrous Na₂SO₄, DCM was removed, and the residue was by purified by HPLC and freeze-dried to get product TASMR_{CTL} with a yield of 58%.

MS of TASMR_{CTL}: m/z 1189.6. ¹H NMR (400 MHz, CDCl₃): δ (ppm): 8.77 (d, J=16, 1H), 7.98 (s, 1H), 7.73-7.62 (m, 3H), 7.54-7.51 (m, 2H), 7.47-7.25 (m, 4H), 7.24-7.19 (m, 5H), 7.04-6.96 (m, 2H), 6.52 (d, J=12, 1H), 5.26 (s, 2H), 4.63 (t, 1H), 4.54-4.51 (m, 1H), 4.42-4.34 (m, 2H), 4.31-4.22 (m, 2H), 4.11 (d, J=8, 1H), 3.46 (t, 2H), 3.21-3.13 (m, 2H), 3.03-2.97 (m, 3H), 2.86-2.8 (m, 3H), 2.78-2.74 (m, 1H), 2.73-2.71 (m, 1H), 2.35-2.28 (m, 2H), 2.19 (t, 1H), 2.04 (s, 3H), 1.97-1.93 (m, 3H), 1.84-1.76 (s, 6H), 1.46-1.4 (m, 3H), 1.48 (s, 3H), 1.3-1.19 (m, 2H), 0.96-0.94 (m, 6H).

### Synthesis of TASMR_{NE}

CyA (10 mg, 0.016 mmol), MeOSuc-AAPV-OH (15.0 mg, 0.032 mmol), HBTU (12.1 mg, 0.032 mmol), HOBt (4.3 mmg, 0.032 mmol), and DIPEA (4.1 mg, 0.032 mmol) were dissolved in DMF (5 mL). The reaction mixture was stirred at 25° C. for 2 h. Then, the reaction mixture was purified by HPLC and freeze-dried to get TASMR_{NE} with a yield of 91%.

¹H NMR (400 MHz, CDCl₃): δ (ppm): 8.78 (d, J=12, 1H), 7.71-7.67 (m, 4H), 7.51-7.42 (m, 6H), 7.1 (t, 2H), 6.53 (d, J=12, 1H), 5.25 (s, 2H), 4.55-4.26 (m, 7H), 3.71-3.57 (m, 2H), 3.5-3.45 (m, 2H), 3.41 (t, 3H), 2.99-2.72 (m, 4H), 2.6-2.5 (m, 4H), 2.22-2.1 (m, 3H), 2.0-1.95 (m, 5H), 1.82 (s, 6H), 1.45-1.4 (m, 2H), 1.35-1.27 (m, 9H), 0.96-0.94 (m, 6H).

Synthesis of TAMR_{M1}, TAMR_{CTL} and TAMR_{NE}

TASMR_{M1} (12 mg, 0.01 mmol) or TASMR_{CTL} (12 mg, 0.01 mmol) or TASMR_{NE} (11 mg, 0.01 mmol) and PVP-alkyne (30 mg, 0.01 mmol) were dissolved in DMSO (1.5 mL). Then, CuSO₄·5H₂O (2.5 mg, 0.01 mmol) and sodium ascorbate (4 mg, 0.02 mmol) in deionized water (1.5 mL) were added. The reaction mixture was stirred at 25° C. for 12 h. Then, the reaction solution was dialyzed against deionized water for 24 h to remove salts and DMSO, and freeze-dried to get TAMR_{M1}, TAMR_{CTL} and TAMR_{NE} with yields of 77%, 92% and 88%, respectively.

TAMR_{M1}. ¹H NMR (400 MHz, CDCl₃): δ (ppm): 8.76 (d, J=12, 1H), 8.00 (m, 3H), 7.82 (s, 1H), 7.78-7.72 (m, 3H), 7.64-7.56 (m, 3H), 7.50-7.42 (m, 5H), 7.09-6.98 (m, 4H), 6.70 (d, J=16, 2H), 6.55 (d, J=16, 1H), 5.28 (s, 2H), 4.59-4.55 (m, 1H), 4.41-4.35 (m, 3H), 4.33-4.24 (m, 2H), 4.15 (s, 2H), 4.11 (t, 1H), 3.92-3.74 (m, 32H), 3.46 (t, 2H), 3.29 (m, 54H), 3.15-3.08 (m, 2H), 2.95-2.91 (m, 1H), 2.83-2.78 (m, 2H), 2.76-2.72 (m, 3H), 2.65-2.59 (m, 1H), 2.38-2.27 (m, 54H), 2.23-2.18 (m, 2H), 2.08-2.03 (m, 58H), 1.93 (s, 3H), 1.83 (s, 6H), 1.75-1.46 (m, 60H), 1.45 (d, J=8, 3H), 1.37 (d, J=8, 3H), 1.02-0.9 (m, 6H).

TAMR_{CTL}. ¹H NMR (400 MHz, CDCl₃): δ (ppm): 8.77 (d, J=16, 1H), 7.98 (s, 1H), 7.82 (s, 1H), 7.73-7.62 (m, 3H), 7.54-7.51 (m, 2H), 7.47-7.25 (m, 4H), 7.24-7.19 (m, 5H), 7.04-6.96 (m, 2H), 6.52 (d, J=12, 1H), 5.26 (s, 2H), 4.63 (t, 1H), 4.54-4.51 (m, 1H), 4.42-4.34 (m, 2H), 4.31-4.22 (m, 2H), 4.15 (s, 2H), 4.11 (d, J=8, 1H), 3.92-3.74 (m, 32H), 3.46 (t, 2H), 3.29 (m, 54H), 3.21-3.13 (m, 2H), 3.03-2.97 (m, 3H), 2.86-2.8 (m, 3H), 2.78-2.74 (m, 1H), 2.73-2.71 (m, 1H), 2.35-2.28 (m, 56H), 2.19 (t, 1H), 2.06-2.01 (m, 57H), 1.97-1.93 (m, 3H), 1.84-1.76 (s, 6H), 1.75-1.46 (m, 60H), 1.46-1.4 (m, 3H), 1.48 (s, 3H), 1.3-1.19 (m, 2H), 0.96-0.94 (m, 6H).

TAMR_{NE}. ¹H NMR (400 MHz, CDCl₃): δ (ppm): 8.78 (d, J=12, 1H), 7.82 (s, 1H), 7.71-7.67 (m, 4H), 7.51-7.42 (m, 6H), 7.1 (t, 2H), 6.53 (d, J=12, 1H), 5.25 (s, 2H), 4.55-4.26 (m, 7H), 4.15 (s, 2H), 3.92-3.74 (m, 32H), 3.71-3.57 (m, 2H), 3.5-3.45 (m, 2H), 3.41 (t, 3H), 3.29 (m, 54H), 2.99-2.72 (m, 4H), 2.6-2.5 (m, 4H), 2.38-2.27 (m, 54H), 2.22-2.1 (m, 3H), 2.06-2.01 (m, 54H), 2.0-1.95 (m, 5H), 1.82 (s, 6H), 1.75-1.46 (m, 60H), 1.45-1.4 (m, 2H), 1.35-1.27 (m, 9H), 0.96-0.94 (m, 6H).

### Synthesis of PEG-Cy

CyOHN₃ (6 mg, 0.01 mmol) and PEG-alkyne (20 mg, 0.01 mmol) were dissolved in DMSO (1.5 mL). Then, CuSO₄·5H₂O (2.5 mg, 0.01 mmol) and sodium ascorbate (4 mg, 0.02 mmol) in deionized water (1.5 mL) were added. The reaction mixture was stirred at 25° C. for 12 h. Then the reaction solution was dialyzed against deionized water for 24 h to remove salts and DMSO, and freeze-dried to get PEG-Cy.

### Synthesis of PVP-Cy

CyOHN₃ (6 mg, 0.01 mmol) and PVP-alkyne (30 mg, 0.01 mmol) were dissolved in DMSO (1.5 mL). Then, CuSO₄·5H₂O (2.5 mg, 0.01 mmol) and sodium ascorbate (4 mg, 0.02 mmol) in deionized water (1.5 mL) were added. The reaction mixture was stirred at 25° C. for 12 h. Then the reaction solution was dialyzed against deionized water for 24 h to remove salts and DMSO, and freeze-dried to get PVP-Cy.

### Pharmacokinetic Studies

Mice were intravenously injected with the uncaged PEG-Cy (5 μmol/kg) and PVP-Cy (5 μmol/kg). Blood samples of PEG-Cy injected mice were collected using heparinized capillary tubes at 1, 4, 7, 11, 16, 25, 35, 55, 75, 95, 120 and 150 min post-injection of PEG-Cy. Blood samples of PVP-Cy injected mice were collected using heparinized capillary tubes at 1, 4, 7, 11, 16, 25, 35, 55, 75, 95, 120, 150 and 180 min post-injection of PVP-Cy. The blood samples in heparinized capillary tubes were then centrifuged at 3500 rpm for 10 min, followed by quantification with HPLC.

### Results and Discussion

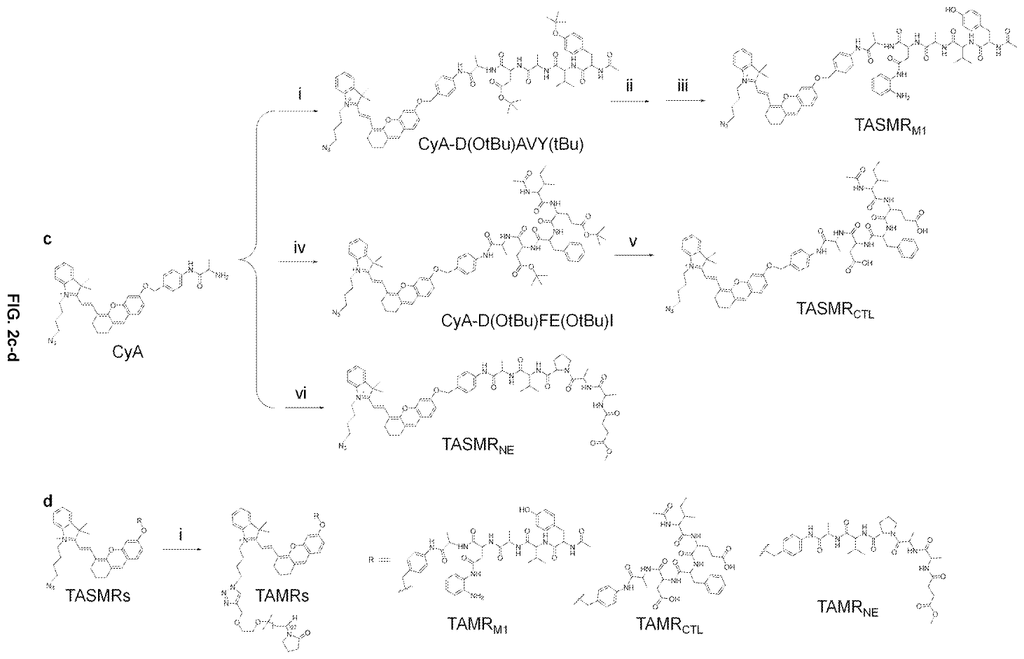

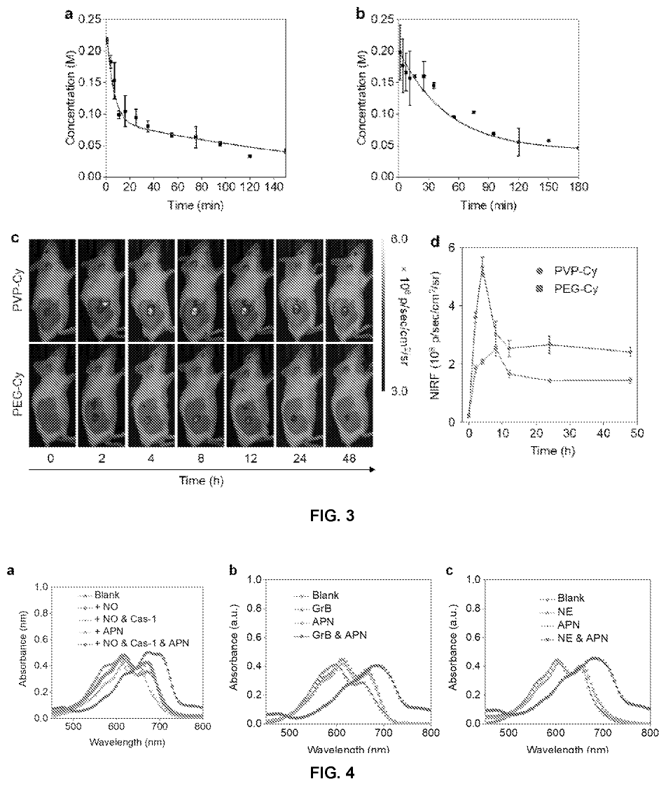

PVP was selected as the tumor-passive targeting moiety because we confirmed that it had higher tumor accumulation efficiency (1.5-fold), and longer circulatory half-life (4.4-fold) in comparison to PEG (FIG. 3).

### Example 2. Characterisation of TAMRs

TAMRs and TASMRs prepared in Example 1 were characterised.

### Optical Measurement

TAMRs (25 μM) were incubated with their respective leukocyte biomarkers or combination of both tumor and respective leukocyte biomarkers (50 μM DEA NONOate, 1 U Cas-1, 0.5 μg APN, 0.5 μg GrB, 2.5 mU NE) in respective buffers at 37° C. for 2 h. For TAMR_{M1}, the enzymatic experiment was conducted in Cas-1 HEPES buffer (50 mM HEPES, pH 7.2, 50 mM NaCl, 0.1% CHAPS, 5% glycerol, 10 mM ethylenediaminetetraacetic acid (EDTA), 10 mM dithiothreitol (DTT)). For TAMR_{CTL}, the enzymatic experiment was conducted in tris buffer (100 mM tris, pH 7.5, 150 mM NaCl). GrB (0.5 μg) was first activated by cathepsin C (0.2 μg) in MES buffer (50 mM MES, pH 5.5, 50 mM NaCl) for 4 h. For TAMR_{NE}, the enzymatic experiment was conducted in NE tris buffer (50 mM Tris, 1 M NaCl, 0.05% (w/v) brij-35, pH 7.5). After completion, UV-Vis absorption and fluorescence spectra of the enzymatic solutions were recorded.

### In Vitro Selectivity Studies

TAMRs or TASMRs (25 μM) were incubated with various enzymes including uPA (0.5 μg), NTR (0.5 μg), GGT (0.5 μg), FAP (0.2 mU), caspase-3 (0.5 μg), CTSS (0.5 μg), GrB (0.5 μg), NE (0.5 mU), APN (0.5 μg), Cas-1 (1 U) and cathepsin C (0.5 μg), and combination of some of these enzymes in their respective buffers at 37° C. for 2 h. For NTR, GGT and caspase-3, the enzymatic experiments were conducted in PBS (10 mM, pH 7.4) buffer. For FAP, the enzymatic experiments were conducted in HEPES buffer (50 mM HEPES, pH 7.4, 0.1% bovine serum albumin (BSA), 5% glycerol). For cathepsin S, the enzymatic experiments were conducted in NaOAc buffer (NaOAc 50 mM, pH 5.5, 5 mM DTT, 250 mM NaCl). For APN, the enzymatic experiments were conducted in tris buffer (50 mM tris, pH 7.0). After completion, the fluorescence intensities of enzymatic solutions were measured by IVIS spectrum imaging system. Excitation: 675 nm. Emission: 720 nm.

### Enzyme Kinetic Assay

Various concentrations of TASMR_{M1} (2, 4, 8, 20, 40, 80, 160 or 200 μM) were incubated with Cas-1 (0.5 U) at 37° C. for 1 h in HEPES buffer (50 mM HEPES, pH=7.2, 50 mM NaCl, 0.1% CHAPS, 10 mM EDTA, 5% glycerol, 10 mM DTT). Various concentrations of TASMR_{CTL} (5, 10, 20, 40, 80, or 120 μM) were incubated with GrB (0.25 μg) at 37° C. for 30 min in tris buffer (100 mM tris, pH 7.5, 150 mM NaCl). Various concentrations of TASMR_{NE} (5, 10, 20, 40, 80, 120, 160 or 200 μM) were incubated with NE (2.5 mU) at 37° C. for 4 min in tris buffer (50 mM Tris, 1 M NaCl, 0.05% (w/v) brij-35, pH 7.5). Various concentrations of CyA (10, 20, 40, 80, 120 or 200 μM) were incubated with APN (0.25 μg) at 37° C. for 20 min in tris buffer (50 mM tris, pH 7.0). After incubation, the mixture was measured by HPLC. The enzymatic reaction velocity (nmol/min or pmol/s) was calculated, plotted as a function of TASMRs or CyA concentrations, and fitted the Michaelis-Menten equation: V=Vmax*[S]/(Km+[S]), where Vmax indicates the maximum theoretical reaction rate, [S] indicates the substrate concentration, and Km indicates the Michaelis constant.

### Results and Discussion

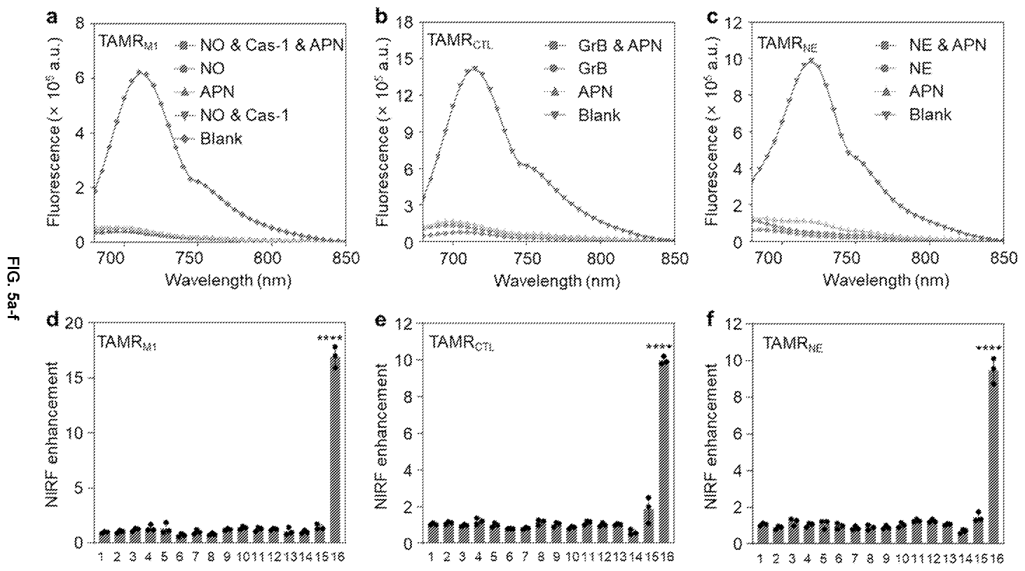

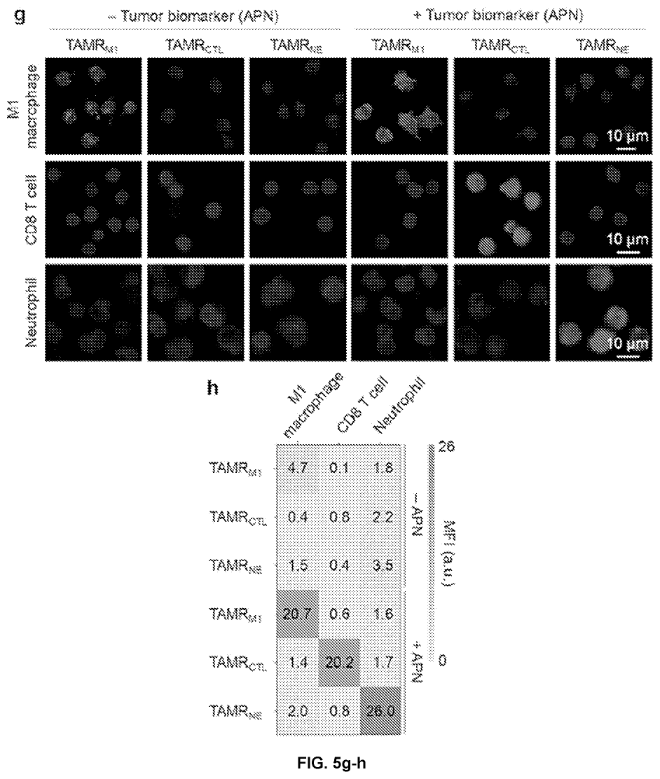

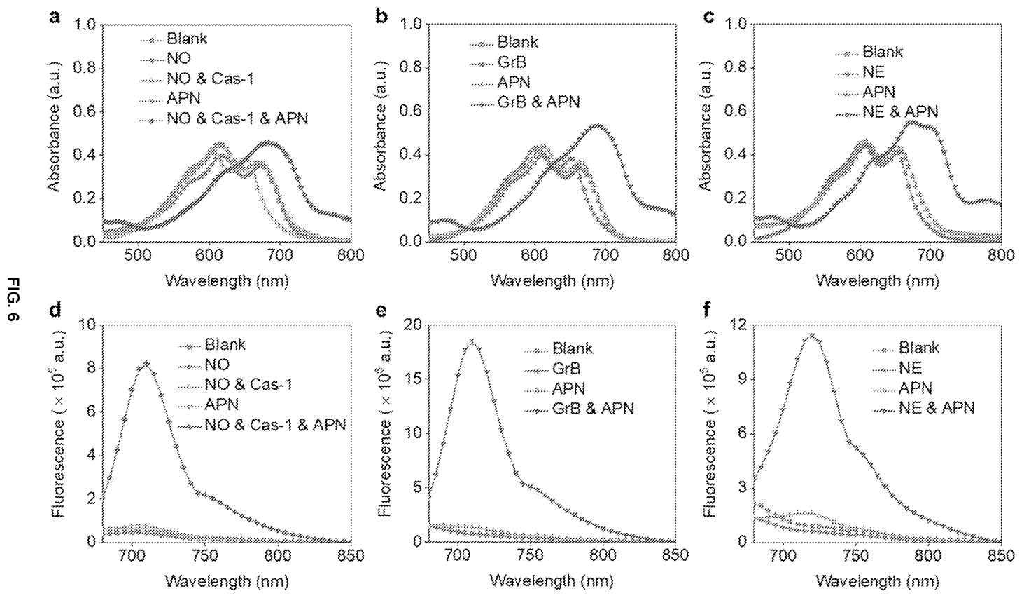

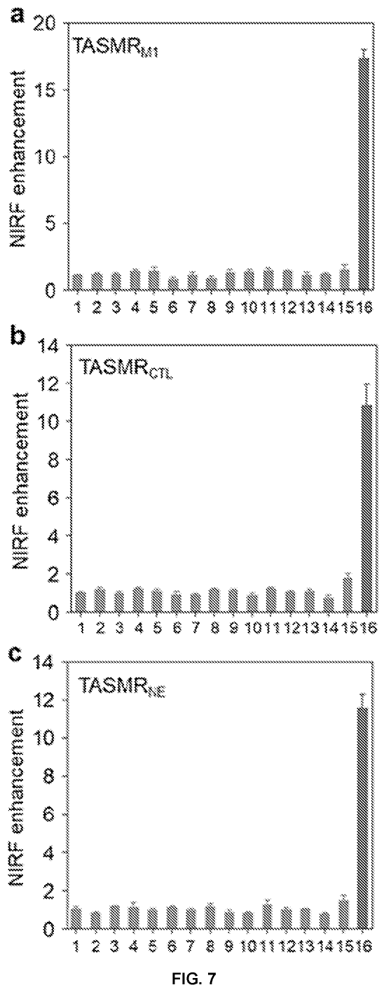

TAMRs(_{M1}, _{CTL}, _{NE}) exhibited similar optical properties with absorption peaks at˜610 and 660 nm, respectively (FIG. 4), and were initially non-fluorescent. Only in the presence of both tumor and respective leukocyte biomarkers, the absorption of TAMRs at 610 nm decreased with the emergence of a new peak at ˜680 nm, which was assigned to the uncaged CyOHP; moreover, the fluorescence at ˜710 nm increased by 12 to 18-fold (FIGS. 5a-c). In comparison, no absorption and fluorescence changes occurred after incubation with either single biomarker. Similar optical profiles were observed for TASMRs (FIG. 6). More importantly, the fluorescence intensities of TAMRs and TASMRs showed negligible enhancement after incubation with other interfering enzymes (FIGS. 5d-f and 7), suggesting their high specificity.

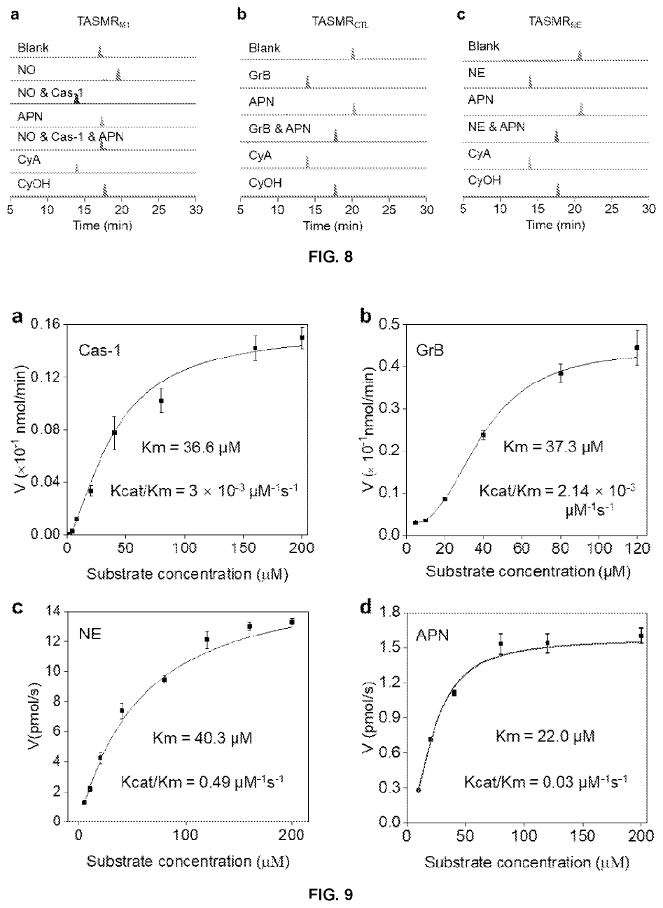

HPLC analysis was applied to investigate the structural changes of TASMRs (TAMR precursors) in response to their respective biomarkers. TASMRs instead of TAMRs were used for the study because HPLC traces of TAMRs remained similar before and after enzymatic activation due to the existence of PVP that dominated the elution property. Incubation of TASMRs with both cancer and leukocyte biomarkers resulted in the appearance of the elution peak assigned to CyOH (T_{R}=17.8 min), which was undetectable after incubation with either single biomarker (FIG. 8). The catalytic efficiencies (Kcat/Km) of Cas-1, GrB and NE towards their respective TASMRs were determined to be 0.003, 0.002, and 0.49 μM⁻¹s⁻¹ (FIGS. 9a-c and Table 1). CyA, the product of TASMRs after cleavage by respective leukocyte biomarkers was further cleaved by cancer biomarker, APN, and the catalytic efficiency of APN towards CyA was calculated to be 0.03 μM⁻¹s⁻¹ (FIG. 9d and Table 1).

### Example 3. Capabilities of TAMRs to Detect TILs

The capabilities of TAMRs (prepared in Example 1) to detect TILs were tested against respective leukocytes in the presence or absence of cancer biomarker (APN, Pasqualini, R. et al., Cancer Res. 2000, 60, 722-727).

### Cell Viability Test

4T1 cells, CT26 cells, M1 macrophages, CD8 T cells and MPRO cells were placed in 24-well plates with 8000 cells per well, and cultured for 24 h, followed by incubation with TAMRs at a final concentration from 12.5 to 100 μg/mL for 24 h. The cells in each well were collected and centrifuged to remove TAMRs, and resuspended in MTS solution (200 μL, 0.1 mg/mL), and incubated for another 4 h at 37° C. The absorbance of MTS solution was measured via a microplate reader (SpectraMax M5 microplate reader) at 490 nm. Cell viability was calculated by the ratio of the absorbance of the cells incubated with TAMRs to that of the respective control cells.

### Cellular Imaging of TAMRs

For cellular imaging of TAMRs, 4T1 cells, CT26 cells, 3T3 cells, M1 macrophages, M2 macrophages, RAW264.7 cells, BMDCs, CD8 T cells and MPRO cells were seeded into confocal cell culture dishes (5×104 cells/dish). After 24 h incubation, the cells were replaced with fresh medium containing TAMRs (10 μM) in the absence or presence of APN (0.5 μg). After incubation for 2 h, cells were washed with PBS for three times, followed by fixation with 4% paraformaldehyde (PFA) for 20 min. Then, the cells were stained with DAPI for the nucleus. Fluorescence images of cells were taken via LSM800 (Zeiss). The fluorescence intensity was quantified by Image J.

### Results and Discussion

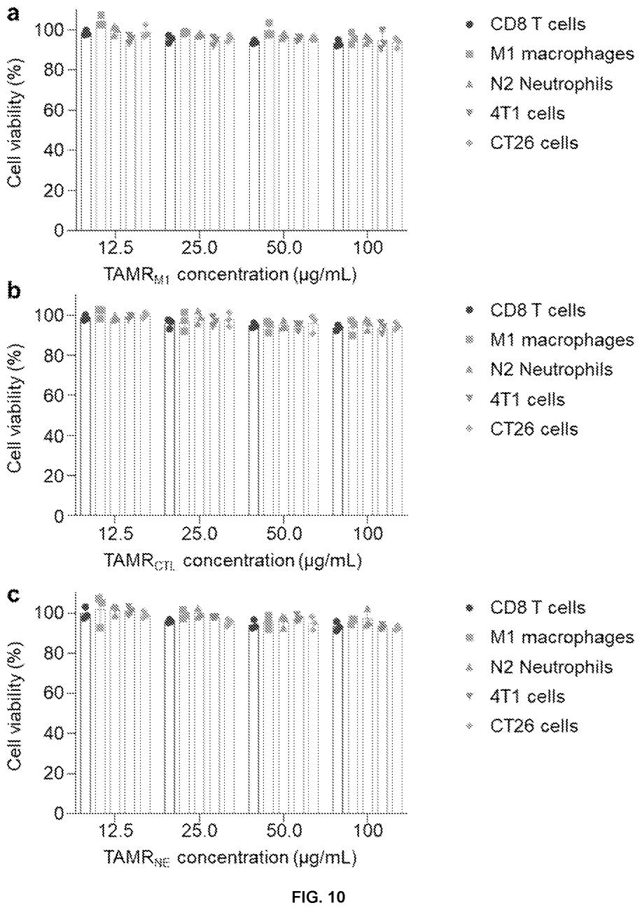

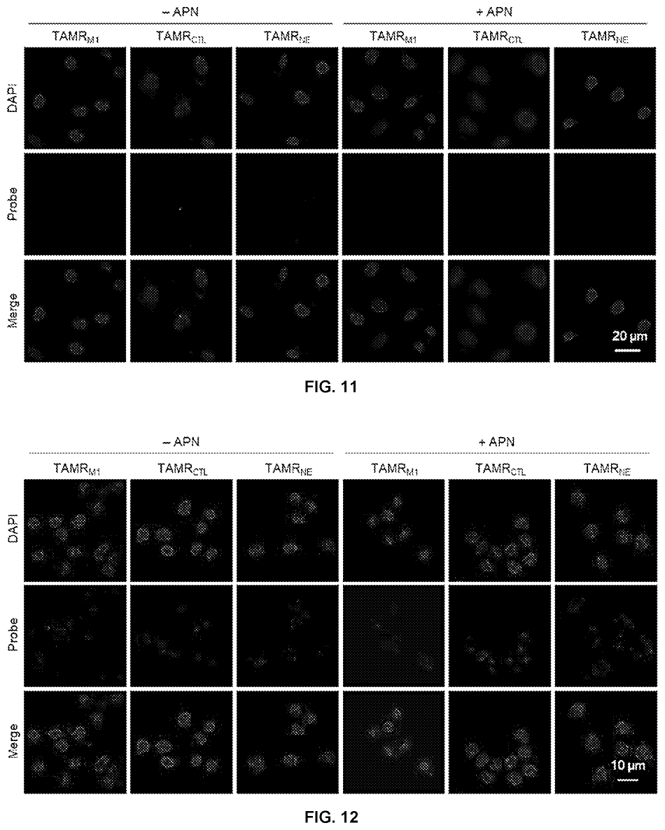

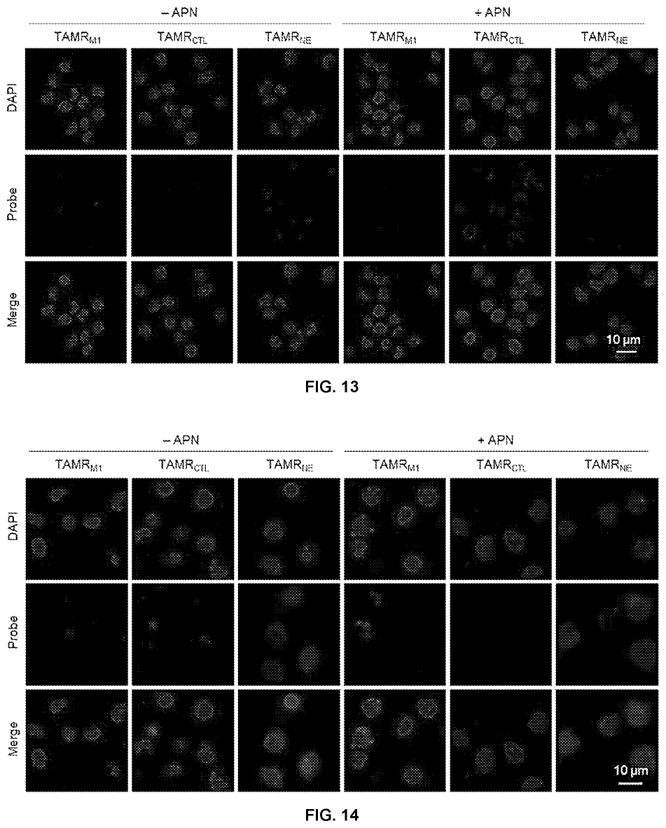

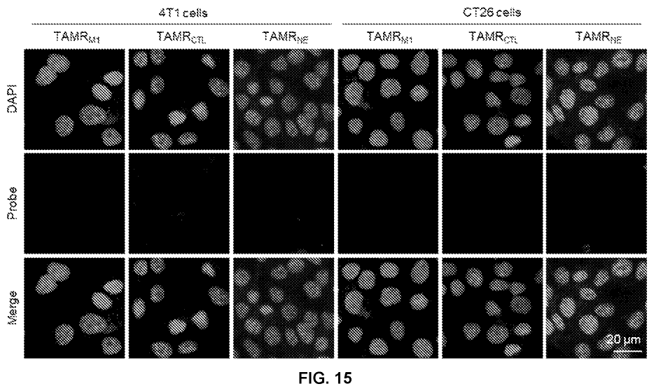

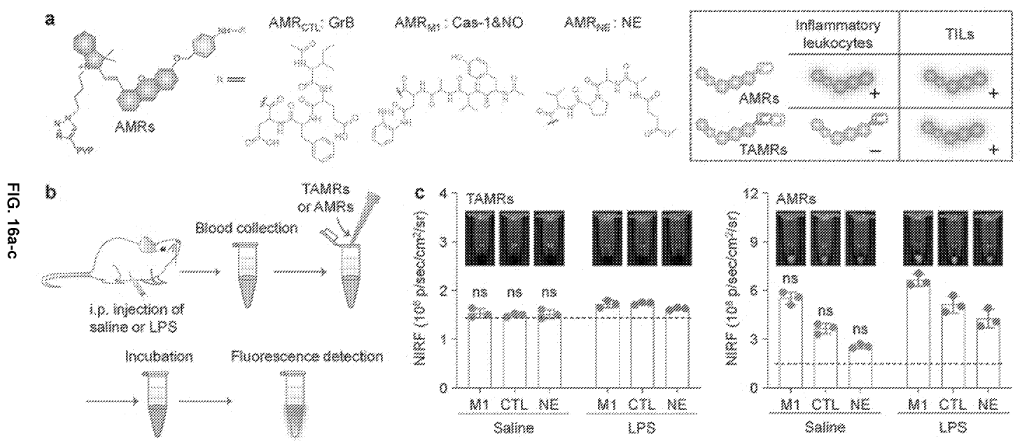

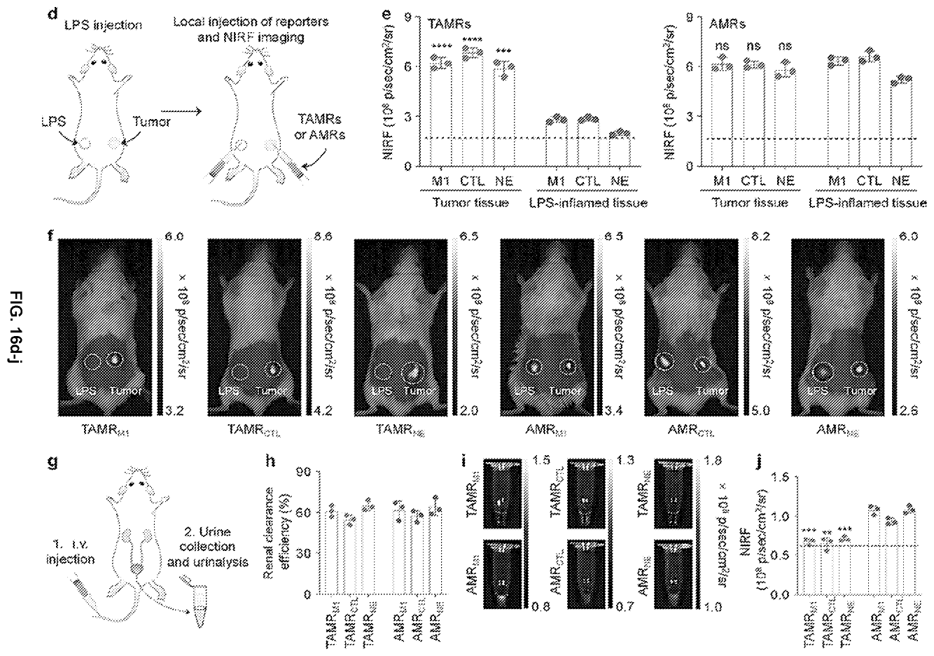

Note that all the TAMRs showed negligible cytotoxicity against selective cells in TIME (FIG. 10). When incubated with various leukocytes in the absence of APN, TAMRs exhibited negligible fluorescence signals. Only in the presence of APN, the NIR fluorescence of TAMRs was turned on in their target leukocytes, which was 8 to 10-fold higher than non-specific leukocytes (FIGS. 5g,h and 11-15). These data further prove that fluorescence activation of TAMRs required both cancer and leukocyte biomarkers, ensuring their specificity towards TILs.

### Example 4. Synthesis of AMRs

AMRs precursors (Cy-DAVY, Cy-DFEI and Cy-VPAA) were synthesized according to previous protocols (He, S. et al., J. Am. Chem. Soc. 2020, 142, 7075-7082). AMRs were synthesized by following the protocol for TAMRs in Example 1.

### Cy-DAVY

MS of Cy-DAVY: m/z 1062.61. ¹H NMR (400 MHz, CDCl₃): δ (ppm): 8.77 (d, J=14, 1H), 7.76-7.71 (m, 3H), 7.58-7.55 (m, 2H), 7.50-7.41 (m, 4H), 7.39-7.33 (m, 1H), 7.06 (d, J=8, 4H), 6.70 (d, J=8, 2H), 6.53 (d, J=12, 1H), 5.25 (s, 2H), 4.76 (t, 1H), 4.59-4.55 (m, 1H), 4.36 (t, 2H), 4.27-4.24 (m, 1H), 4.10 (t, 1H), 3.46 (t, 2H), 3.28-3.10 (m, 1H), 3.05-3.0 (m, 1H), 2.96 (t, 1H), 2.91-2.85 (m, 2H), 2.79-2.72 (m, 2H), 2.74-2.71 (m, 2H), 2.08-2.03 (m, 2H), 2.0-1.97 (m, 3H), 1.91 (s, 3H), 1.83 (s, 6H), 1.71 (m, 1H), 1.36 (d, J=8, 3H), 1.02-0.9 (m, 6H).

### Cy-DFEI

MS of Cy-DFEI: m/z 1118.58. ¹H NMR (400 MHz, CDCl₃) of Cy-DFEI: δ (ppm): 8.76 (d, J=16, 1H), 7.75-7.72 (m, 3H), 7.56 (d, J=4, 2H), 7.50-7.47 (m, 4H), 7.41 (s, 1H), 7.23-7.16 (m, 4H), 7.12-7.07 (m, 3H), 6.53 (d, J=16, 1H), 5.28 (s, 2H), 4.8 (t, 1H), 4.54-4.51 (m, 1H), 4.38 (t, 2H), 4.26-4.23 (m, 1H), 4.13 (d, J=8, 1H), 3.48 (t, 2H), 3.37 (s, 2H), 3.20-3.15 (m, 1H), 3.06-2.96 (m, 2H), 2.79-2.73 (m, 5H), 2.34-2.23 (m, 2H), 2.04 (s, 3H), 1.97-1.93 (m, 4H), 1.84-1.76 (s, 6H), 1.46-1.4 (m, 3H), 1.3-1.19 (m, 2H), 0.90-0.88 (m, 6H).

### Cy-VPAA

¹H NMR (400 MHz, CDCl₃): δ (ppm): 8.80 (d, J=16, 1H), 7.75-7.73 (m, 2H), 7.64-7.62 (m, 2H), 7.57-7.52 (m, 2H), 7.49-7.43 (m, 2H), 7.35-7.27 (m, 2H), 7.13-7.06 (m, 2H), 6.55 (d, J=16, 1H), 5.25 (s, 2H), 4.39 (t, 1), 4.29 (t, 3H), 4.23-4.17 (m, 2H), 3.83-3.81 (m, 2H), 3.67-3.62 (m, 2H), 3.54-3.46 (m, 2H), 3.24-3.15 (m, 1H), 2.82 (s, 2H), 2.75 (t, 1H), 2.72 (s, 2H), 2.68-2.65 (m, 2H), 2.53 (d, J=8, 1H), 2.06-1.94 (m, 4H), 1.66-1.59 (m, 2H), 1.82 (s, 6H), 1.45-1.4 (m, 2H), 1.36-1.34 (m, 6H), 1.22-1.15 (m, 2H), 0.96-0.94 (m, 6H).

### Example 5. In Vivo Specificity of TAMRs Towards TILs

The development of imaging agents with high specificity for TILs remains challenging because leukocytes exist in peripheral blood and inflamed tissues (Nourshargh, S. & Alon, R., Immunity 2014, 41, 694-707). The specificity of TAMRs (prepared in Example 1) towards TILs was investigated and compared with their single-lock counterparts (AMRs, prepared in Example 4) (FIG. 16a). Blood samples of saline or LPS-treated mice were incubated with these reporters for NIRF imaging (FIG. 16b).

### Absorption Analysis

LPS (5.0 mg/kg) was intraperitoneally injected into living mice, and the LPS-inflamed blood samples were collected 4 h post-injection of LPS. TAMRs and AMRs (10 μM) were respectively incubated with LPS-inflamed and saline-treated blood samples for 30 min. Then, the blood samples were homogenized and centrifuged for 10 min. The supernatants were used for absorption analysis.

### In Vivo Specificity Detection

For blood incubation experiments, blood samples were first obtained from BALB/c mice. Briefly, saline or LPS (5 mg/kg mice) was intraperitoneally injected into living mice. After 4 h, the blood samples were collected. TAMRs (final concentration: 10 μM) were added into saline-treated and LPS-inflamed blood samples (50 μL). After 30 min, fluorescence images of blood samples were acquired by IVIS spectrum imaging system. Excitation: 675 nm. Emission: 720 nm.

For in vivo specificity detection, living mice bearing LPS-inflamed tissue in the left thigh muscle and subcutaneous CT26 tumor on the right flank were first built. Briefly, CT26 tumors were first inoculated in the right flank. 7 days later, LPS (5 mg/kg) were injected into the left thigh muscle. 24 h later, 10 μL of TAMRs or AMRs (0.1 mM) were locally injected into both LPS-inflamed tissues and CT26 tumors for longitudinal NIRF imaging. NIRF images were acquired by IVIS spectrum imaging system. Excitation: 675 nm. Emission: 720 nm.

### Results and Discussion

The fundamental challenge in molecular imaging of TILs lies in the lack of probe design to distinguish TILs from resident leukocytes in other organs. This challenge is tackled by our dual-locked tandem molecular design that simultaneously incorporate both disease-site and biomarker specificities into signal activation of probes. The dual-locked TAMRs only triggered their fluorescence in the presence of both cancer and leukocyte biomarkers, and thus specifically detected TILs with no false positives from other leukocytes in LPS-induced inflammation; in contrast, their single-lock counterparts failed to do so (FIG. 16).

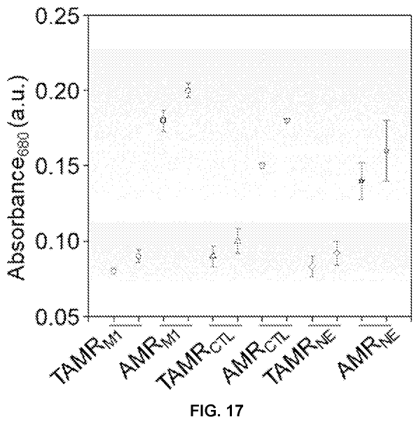

In comparison to their pure forms in PBS solution, TAMRs exhibited almost identical NIRF signals in the saline-treated blood samples, and slightly increased signals in LPS-inflamed blood samples (1.1 to 1.2-fold). In contrast, AMRs showed 1.8 to 3.6-fold, and 2.8 to 4.9-fold higher signals in saline-treated and LPS-inflamed blood samples relative to their NIRF signals in PBS, respectively (FIG. 16c). The activation of AMRs was further confirmed by their higher absorption intensities at 680 nm in the blood samples; 1.7 to 2.5-fold higher than that of TAMRs (FIG. 17).

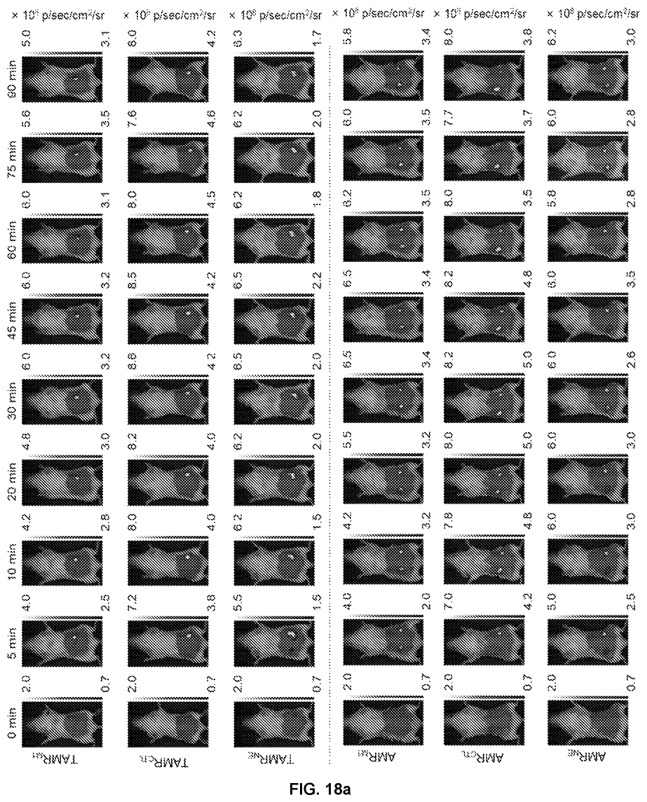

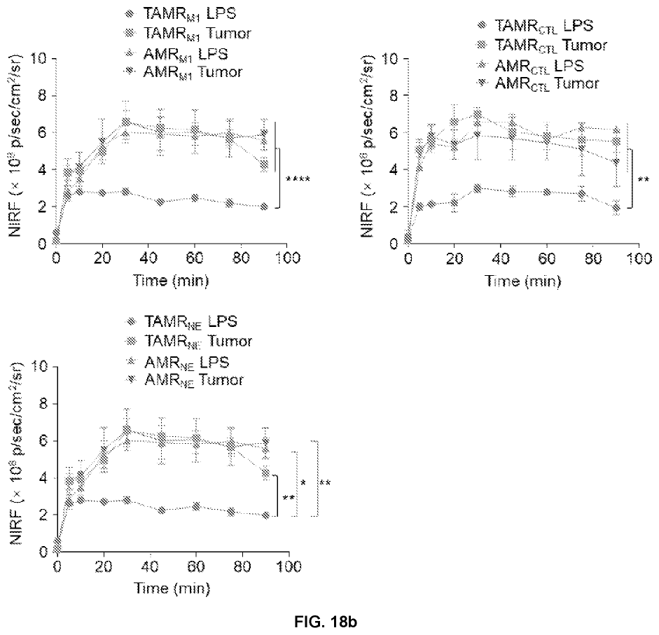

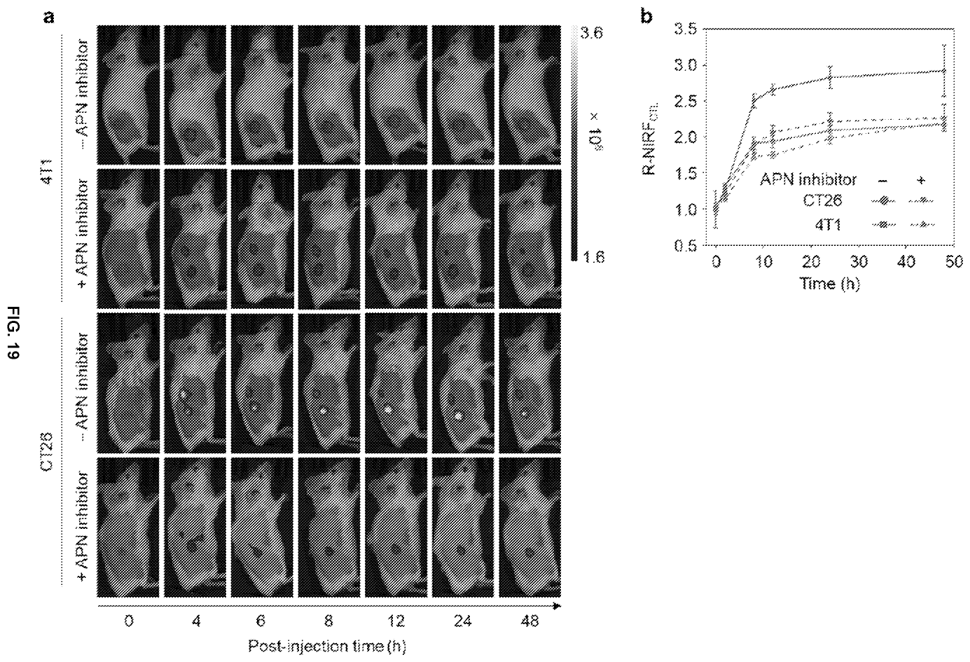

The specificity of TAMRs towards TILs was further verified in living mice bearing LPS-inflamed tissue in the left thigh muscle (Ning, X. et al., Nat. Mater. 2011, 10, 602-607) and subcutaneous CT26 tumor on the right flank (FIG. 16d). Local injection of TAMRs or AMRs presented a signal increase reaching maximum at 30 min post-injection of these reporters in both LPS-inflamed and tumor tissues (FIG. 18). Thus, the signals of reporters in both LPS-inflamed and CT26 tumor tissues at 30 min post-injection of reporters were compared with the background signals of these reporters in the skin. Significant signal enhancement was only observed for TAMRs in tumor tissues (3.6 to 4.2-fold) but not for LPS-inflamed tissues (1.1 to 1.6-fold); in contrast, it was detected for both LPS-inflamed and tumor tissues (3.1 to 4.2-fold) for AMRs (FIGS. 16e,f). In addition, when 4T1 or CT26 tumors were pre-treated with APN inhibitor, bestatin (Shim, J. S. et al., Chem. Biol. 2003, 10, 695-704), the signal of TAMRs at tumor sites was slightly increased by 1.1 to 1.2-fold in comparison to skin background of reporters (FIG. 19), further proving that cancer biomarker was essential for TAMRs activation.

### Example 6. Clearance Pathway and In Vivo Stability of TAMRs

To determine the clearance pathway and in vivo stability of TAMRs, urine of healthy mice injected with TAMRs (prepared in Example 1) or AMRs (prepared in Example 4) was collected and quantified with HPLC (FIG. 16g).

### Renal Clearance Efficiency Studies

Healthy mice were intravenously injected with TAMR_{M1}, TAMR_{CTL}, or TAMR_{NE} (5 μmol/kg) and their single-lock counterparts AMR_{M1}, AMR_{CTL}, or AMR_{NE} (5 μmol/kg), and placed into metabolic cages. Urine samples were collected at 3, 9, and 24 h post-injection of these reporters. The renal clearance of these reporters was examined by HPLC analysis of the urine samples. The urine samples were also centrifuged at 5000 rpm for 15 min, and the UV-Vis absorption spectra of the supernatants were further recorded.

UV-Vis Analysis of Urine Collected from Healthy Mice

Healthy mice were intravenously injected with TAMRs (5 μmol/kg) or AMRs (5 μmol/kg), and placed into metabolic cages. At 12 post-injection timepoint, the urine samples were collected and centrifuged at 5000 rpm for 15 min, and the UV-Vis absorption spectra of the supernatants were further recorded (n=3).

### Results and Discussion

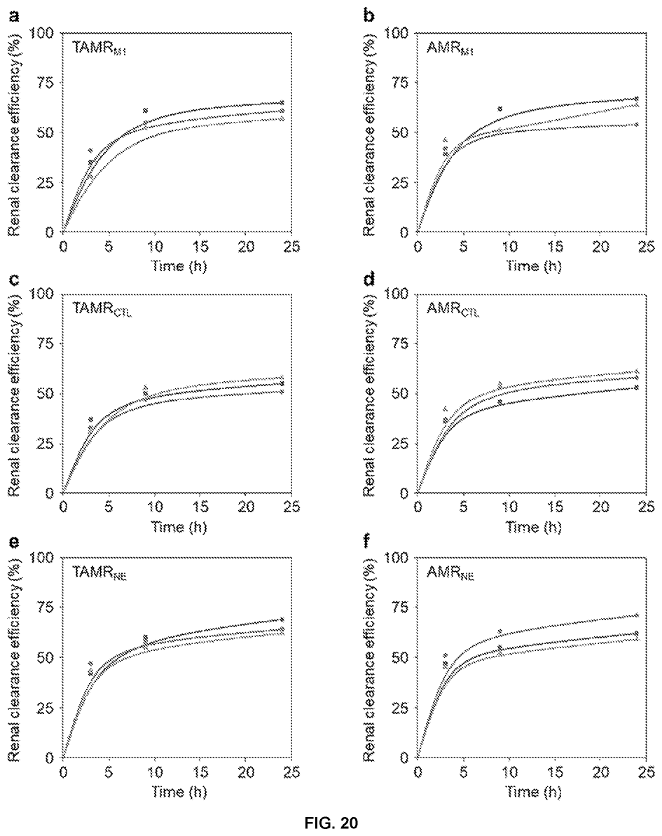

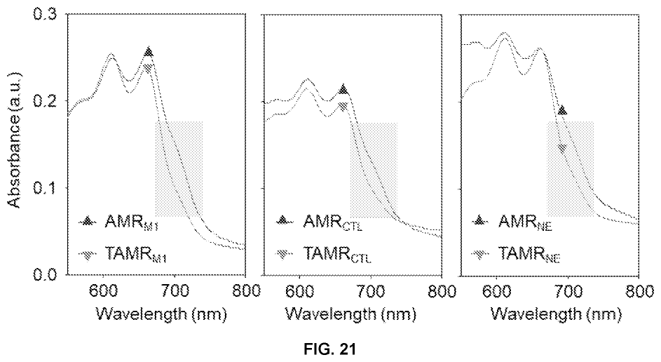

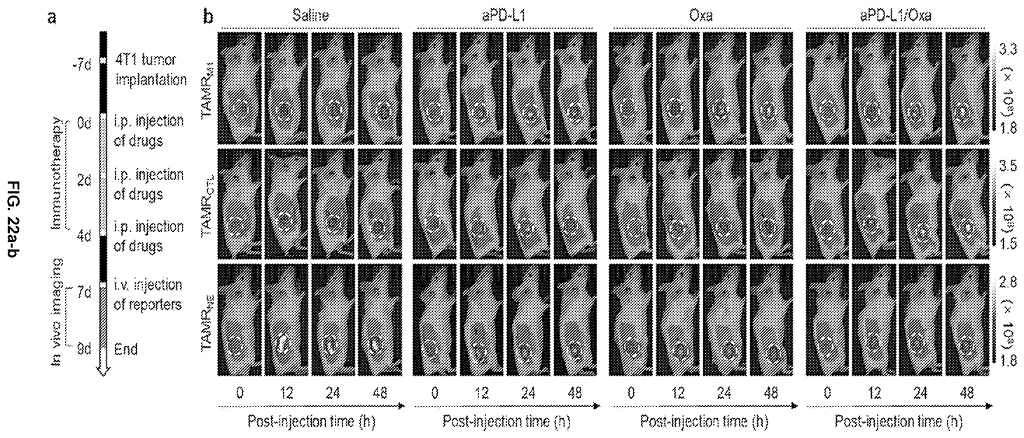

Due to their high-water solubility, the renal clearance efficiency of these reporters at 24 h post-injection was 55-71% of the total injection dosage (FIGS. 16h and 20). The signals of TAMRs in excreted urine remained as low as those from their pure forms in PBS, indicating the high in vivo stability of TAMRs during systematic circulation and excretion. In contrast, the signals of excreted AMRs were 1.6 to 1.8-fold higher than those in PBS (FIGS. 16i,j), and the new absorption peak at 680 nm assigned to activated reporters was observed for excreted AMRs (FIG. 21). These data confirm that dual-lock TAMRs minimized the nonspecific activation by circulating leukocytes, possessing high specificity towards TILs.

Due to the high renal clearance of TAMRs, they can be applied for fluorescence urinalysis of TIME for evaluation of cancer immunotherapy, showing great potential for clinical translation. We report real-time NIRF imaging of TILs using TAMRs for companion diagnosis and prediction of cancer immunotherapy in the following examples.

### Example 7. Real-Time NIRF Imaging of TILs in Poorly Immunogenic Tumor

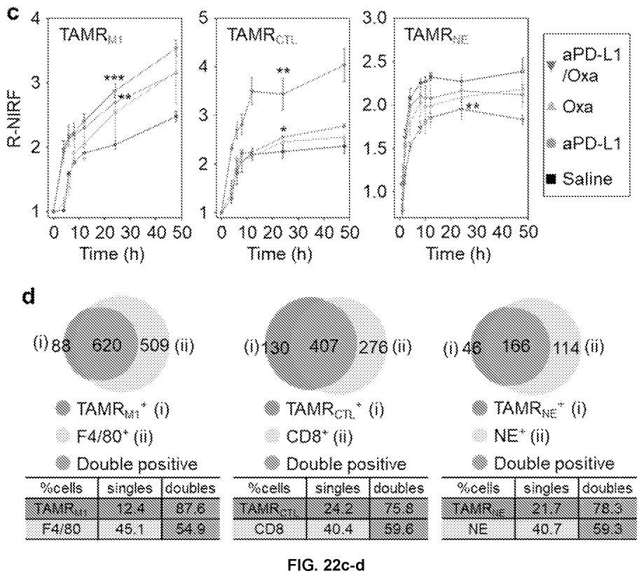

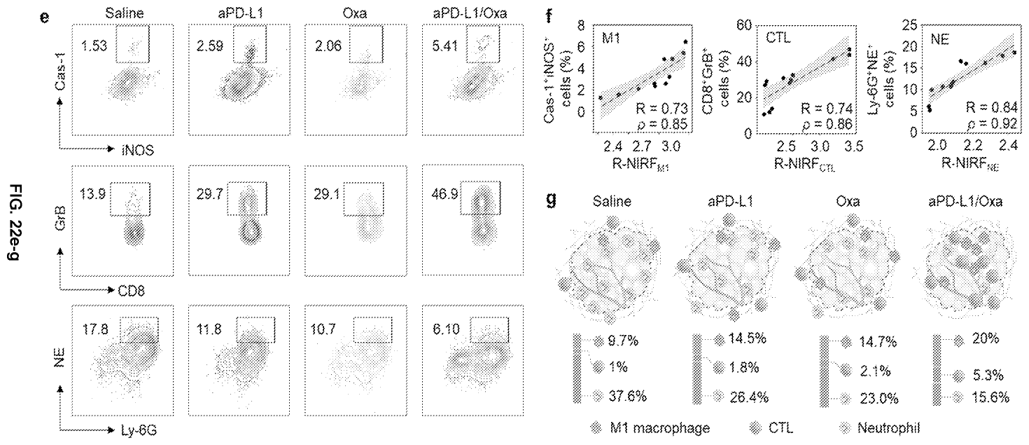

The capabilities of TAMRs (prepared in Example 1) for in vivo real-time imaging of TILs were first validated against mice bearing 4T1 tumor, which has been considered as a poorly immunogenic tumor as it shows a lower presence of tumor-suppressive leukocytes (Taylor, M. A. et al., J. Immunother. Cancer 2019, 7, 328). Mice were administrated with aPD-L1, Oxa or the combination of aPD-L1 and Oxa (aPD-L1/Oxa) intraperitoneally (FIG. 22a). Oxa, the third-generation platinum drug, is known to inhibit cancer growth by inducing immunogenic cell death (ICD) for activating dendritic cells (DCs) and improving T cell infiltration (Zitvogel, L. et al., Nat. Rev. Immunol. 2008, 8, 59-73). Anti-programmed death-ligand 1 (aPD-L1), has been used to inhibit the binding of PD-L136, which was up-regulated in solid tumors, to programmed cell death protein 1 (PD-1) receptor expressed on T cells for immune checkpoint inhibition (ICI), and boost adaptive immune responses against cancer cells.

### In Vivo Real-Time Imaging of TILs

After tumor inoculation for one week, 4T1 tumor-bearing mice were administrated with aPD-L1 (10 mg/kg), Oxa (6 mg/kg) or the combination of aPD-L1 and Oxa (aPD-L1/Oxa) intraperitoneally every two days for three times. CT26 tumor-bearing mice were administrated with aPD-L1 (10 mg/kg), aCD47 (10 mg/kg) or the combination of aPD-L1 and aCD47 (aPD-L1/aCD47) intraperitoneally every two days for three times. At day 7, tumor penetration reference reporter PVP-IR800 (3 μmol/kg) and TAMRs including TAMR_{M1}, TAMR_{CTL} and TAMR_{NE} (5 μmol/kg) were i.v. injected, and real-time NIRF imaging was monitored for 48 h. For APN inhibition group, bestatin (APN inhibitor, 10 mg/mL, 10 μL) was injected intratumorally 2 h before i.v. injection of TAMR_{CTL}. Fluorescence imaging was taken with excitation at 675 nm and emission at 720 nm for monitoring of activated TAMRs, and excitation at 745 nm and emission at 800 nm for monitoring of IR800.

### Ex Vivo Real-Time Imaging of TILs

At 72 h post-injection of PVP-IR800 (3 μmol/kg) and TAMRs (5 μmol/kg), mice were euthanized and major tissues including heart, liver, spleen, lung, kidneys, and tumor were collected and captured by IVIS spectrum imaging system with excitation at 675 nm and emission at 720 nm for monitoring of activated TAMRs, and excitation at 745 nm and emission at 800 nm for monitoring of IR800.

After ex vivo imaging, the major organs were suspended in PBS, homogenized, and centrifuged (10000 rpm, 10 min) to remove insoluble components. The supernatant containing extracted reporters was analyzed by HPLC assay to present the distribution of TAMRs.

### Immunofluorescence Imaging

At 24 h post-injection of TAMRs (5 μmol/kg), mice were euthanized, and tumors were collected, followed by fixation in 4% PFA. After dehydration with 30% sucrose solution, tumor tissues were embedded in O.C.T. medium for 10 min, followed by cutting into 10-μm sections using a cryostat (Leica, CM1950). Tumor sections were washed with PBS containing 0.1% triton X-100 (PBST), followed by incubation with 3% BSA solution at 25° C. for 2 h to block non-specific binding of antibodies. Tumor sections were stained with Alexa Fluor® 488 anti-F4/80 (C-7), PE anti-mouse CD8a, and NE polyclonal antibody for TAMR_{M1}, TAMR_{CTL} and TAMR_{NE} groups, respectively. For TAMR_{NE} group, the tumor sections were further stained with secondary antibody Alexa Fluor® 488 conjugated goat anti-rabbit IgG. Finally, tumors sections were stained with DAPI for the nucleus. The fluorescence images of tumor sections were captured on LSM800 (Zeiss). Finally, the colocalization of NIRF signals of TAMRs with their respective leukocytes was analyzed using ImageJ software.

### Ex Vivo Flowcytometry Analysis of Leukocytes

After three times of immunotherapy, at day 8, both 4T1 and CT26 tumor-bearing mice in each group were euthanized, and tumors, lymph nodes, and blood cells were harvested to prepare single cell suspension. For evaluation of TILs, tumor tissues were cut into small pieces and digested at 37° C. for 4 h in RPMI 1640 containing type I collagenase (1 mg/mL), type IV collagenase (100 μg/mL), and DNase I (100 μg/mL). Then, the mixture was filtered through a 70 μm cell strainer. For evaluation of leukocytes in lymph node and blood cells, cells of lymph nodes and blood cells were treated with ACK lysis to remove red blood cells. All the single cell suspensions were first blocked with anti-mouse CD16/32, followed by live/dead staining. For CTLs analysis, the cells were stained with Alexa Fluor® 700 anti-mouse CD45, FITC anti-mouse CD3 and PE anti-mouse CD8a for 30 min at 4° C., followed by fixation in 4% PFA in the dark for 20 min at 25° C. Then, cells were resuspended in ISPWB and incubated with APC anti-human/mouse GrB recombinant antibody for 1 h at 25° C., followed by washing with ISPWB for three times. For M1 macrophages analysis, the cells were stained with Alexa Fluor® 700 anti-mouse CD45, PerCP anti-mouse/human CD11 b and Alexa Fluor® 488 anti-F4/80 (C-7) for 30 min at 4° C., followed by fixation in 4% PFA in the dark for 20 min at 25° C. Thereafter, cells were resuspended in ISPWB and incubated with PE anti-mouse NOS2 and Alexa Fluor® 647 anti-Cas-1 (D-3) for 1 h at 25° C., followed by washing with ISPWB for three times. For neutrophils analysis, the cells were stained with Alexa Fluor® 700 anti-mouse CD45, PE anti-mouse/human CD11b and APC anti-mouse Ly-6G for 30 min at 4° C., followed by fixation in 4% PFA in the dark for 20 min at 25° C. Thereafter, cells were resuspended in ISPWB and incubated with NE polyclonal antibody for 1 h at 25° C., followed by washing with ISPWB for three times. The cells were then incubated with secondary antibody Alexa Fluor® 488 conjugated goat anti-rabbit IgG for 1 h at 25° C., followed by washing with ISPWB for three times. The final cells were analyzed with Fortessa X20 (BD Biosciences).

### Results and Discussion

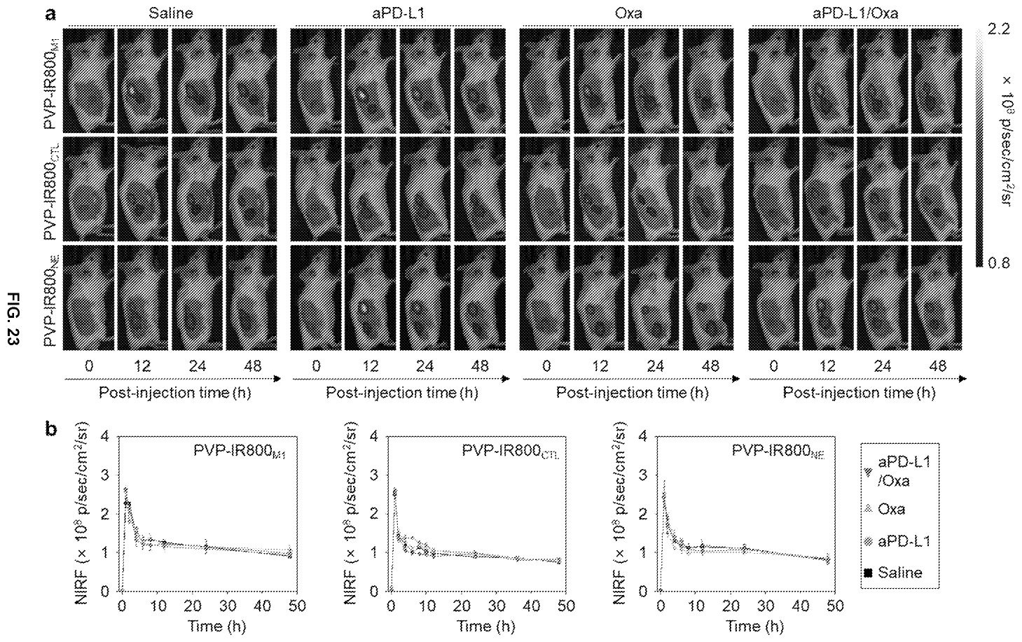

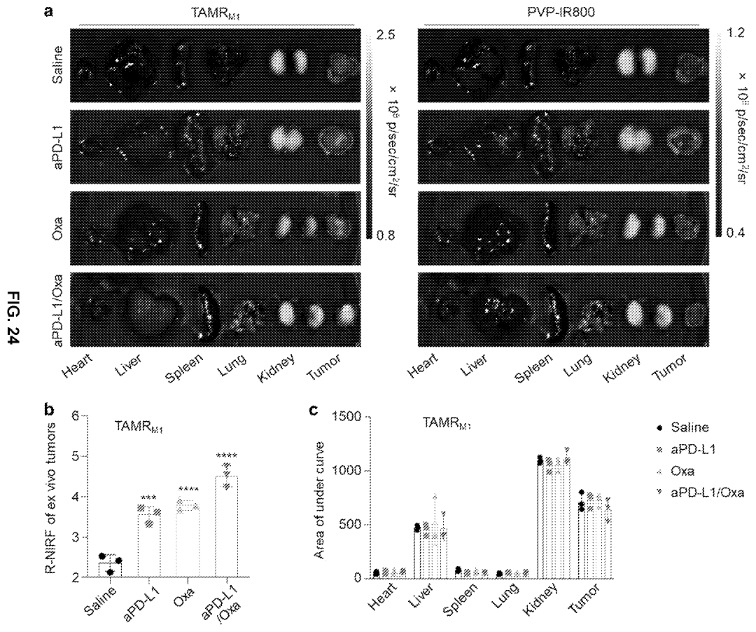

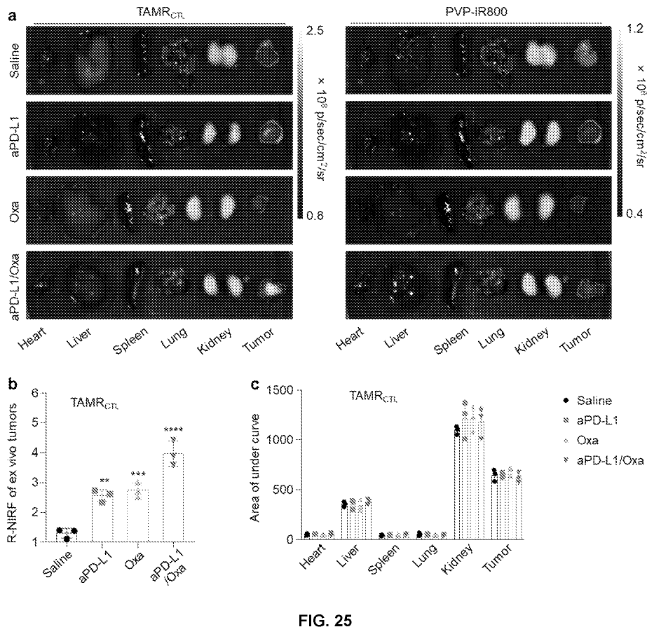

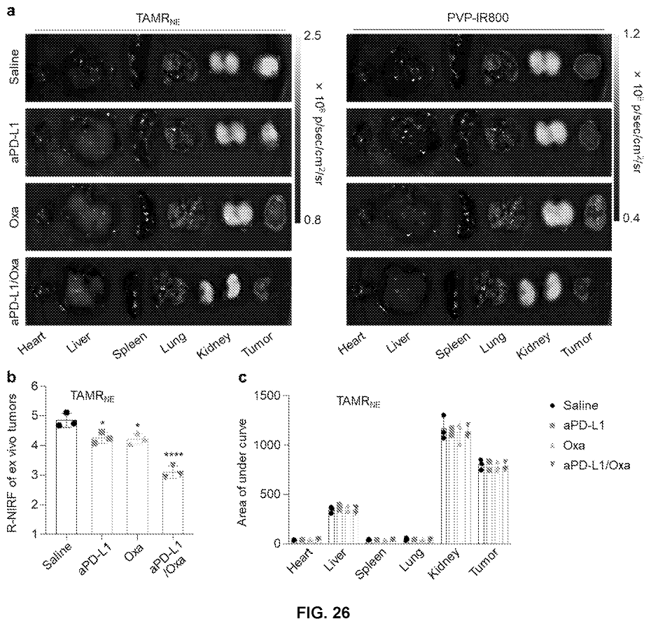

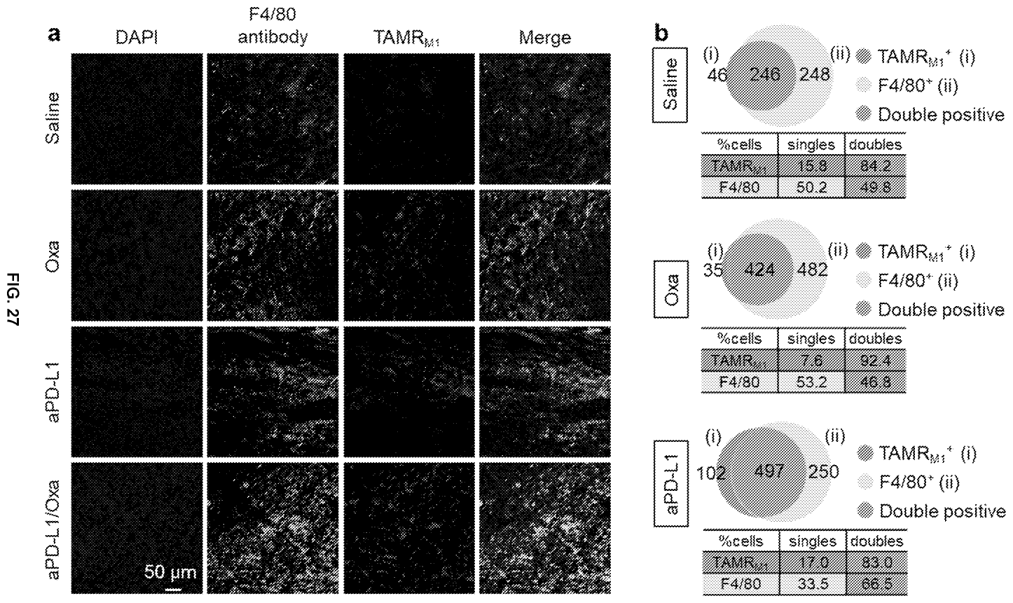

After three times of immunotherapies, TAMRs and the “always-on” reference reporter (PVP-IR800) were intravenously co-injected into 4T1 tumor-bearing mice for longitudinal NIRF imaging (FIG. 22a). The NIRF signals in the tumor regions gradually increased and reached maxima at 24 h post-injection of reporters (FIG. 22b), while NIRF signals of PVP-IR800 reached maxima at 2 h post-injection of reporters (FIG. 23). Thus, the tumor signals for different treatment groups were compared at 24 h post-injection of reporters. To rule out the contribution of concentration difference of TAMRs to the detected signals in tumor, the ratiometric signals of ‘TAMRs’ to ‘PVP-IR800’ were defined and termed as R-NIRF_{M1}, R-NIRF_{CTL} or R-NIRF_{NE}. R-NIRF_{M1} was the highest for aPD-L1/Oxa treated mice, which was 1.1-, 1.2- and 1.5-fold higher than those for aPD-L1, Oxa and untreated mice, respectively. R-NIRF_{CTL} was also the highest for aPD-L1/Oxa treated mice, which was 1.4-, 1.5- and 1.6-fold higher than those for aPD-L1, Oxa and untreated mice, respectively. In contrast, R-NIRF_{NE} was the lowest for aPD-L1/Oxa treated mice, which was 1.1-, 1.1- and 1.2-fold lower than those for aPD-L1, Oxa and untreated mice, respectively (FIGS. 22c and 24-26). These data demonstrate that TAMR_{M1} and TAMR_{CTL} showed the highest activation, while TAMR_{NE} showed the lowest activation in the tumors of aPD-L1/Oxa treated mice.

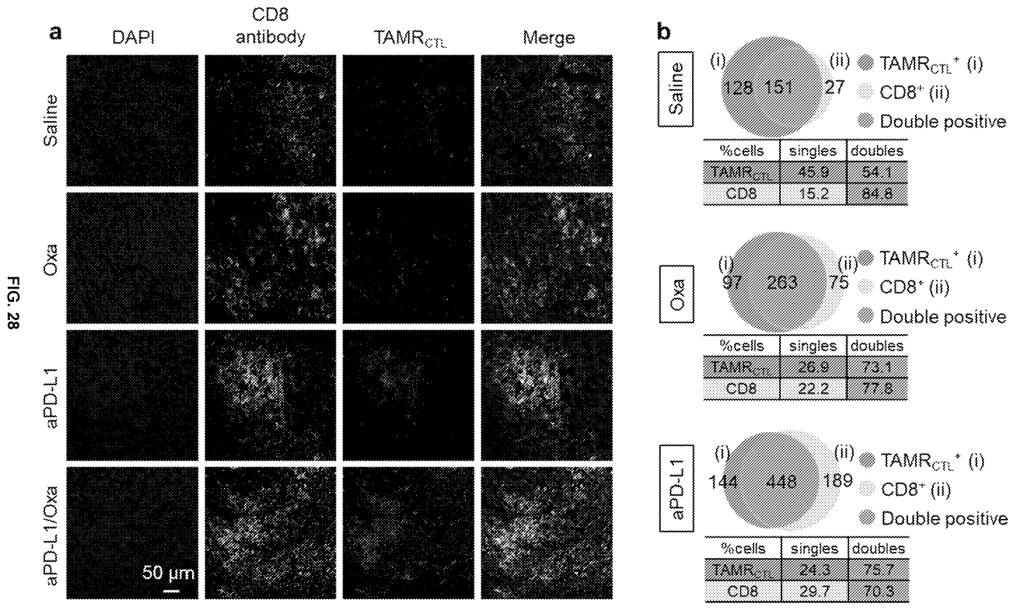

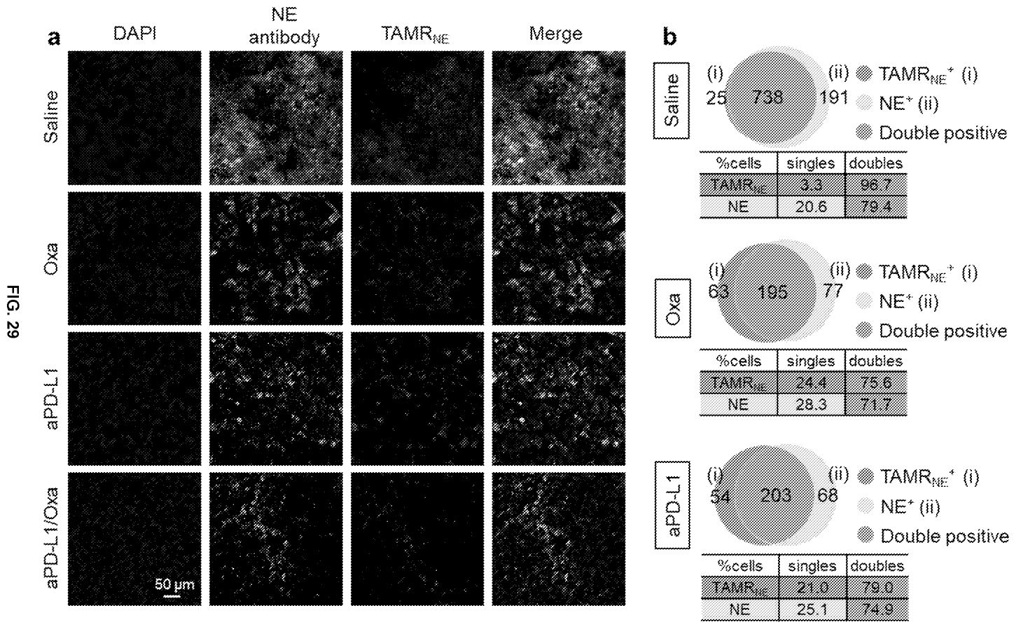

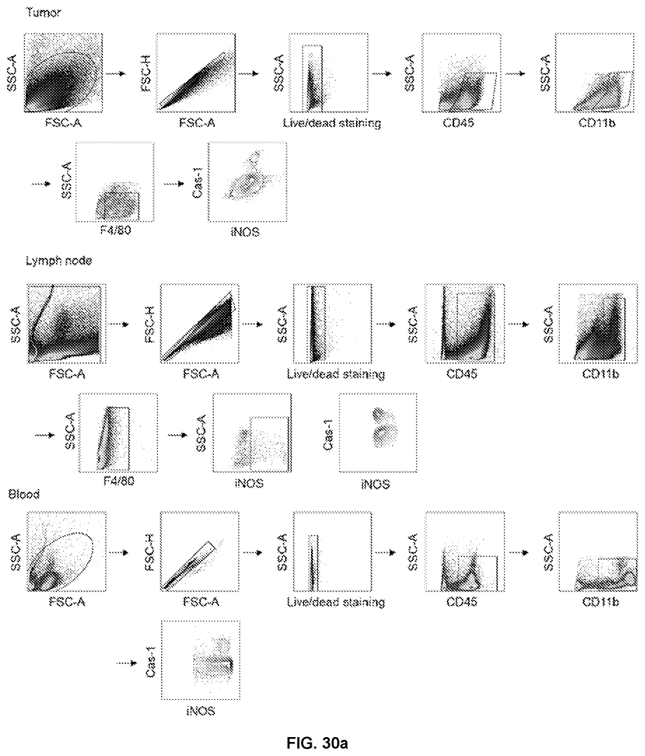

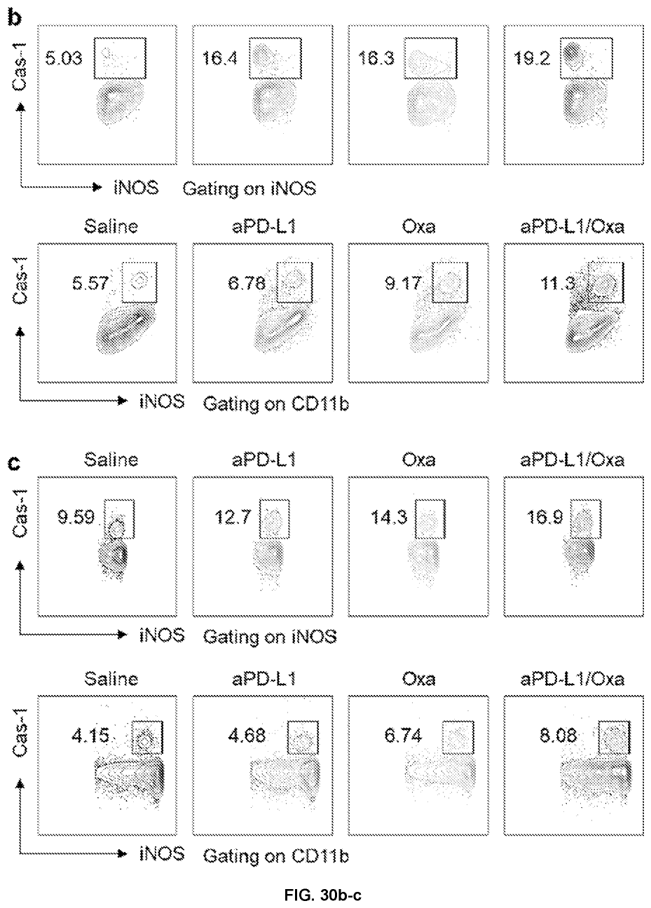

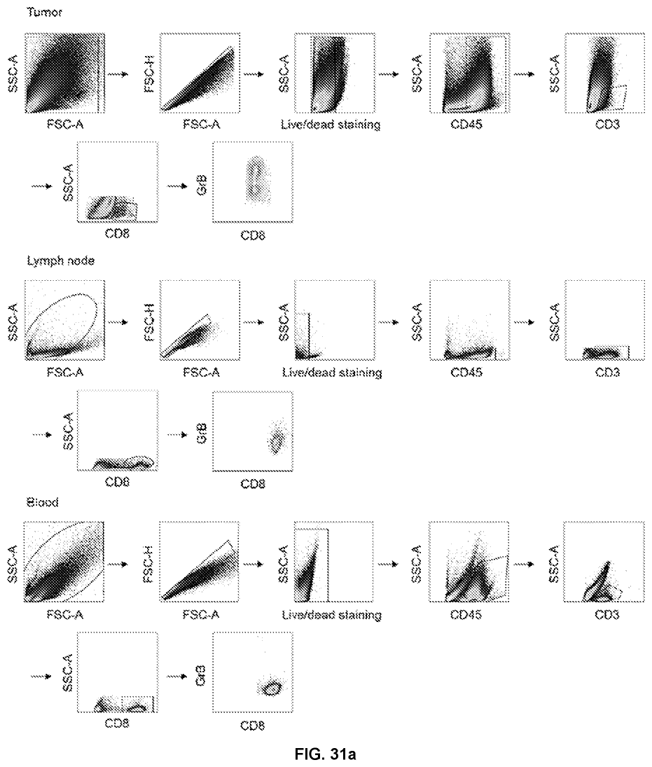

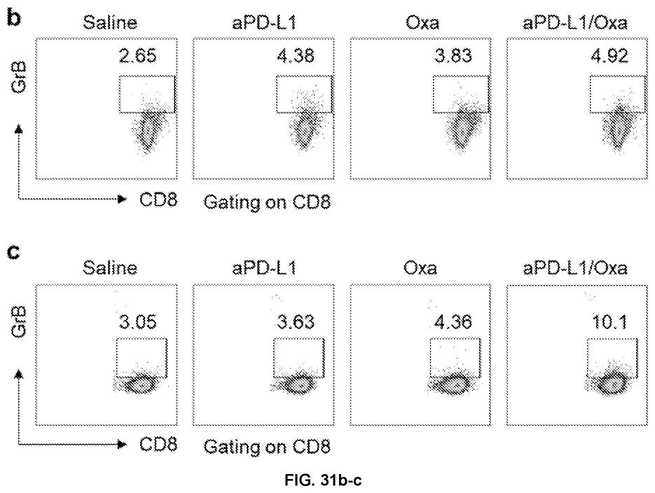

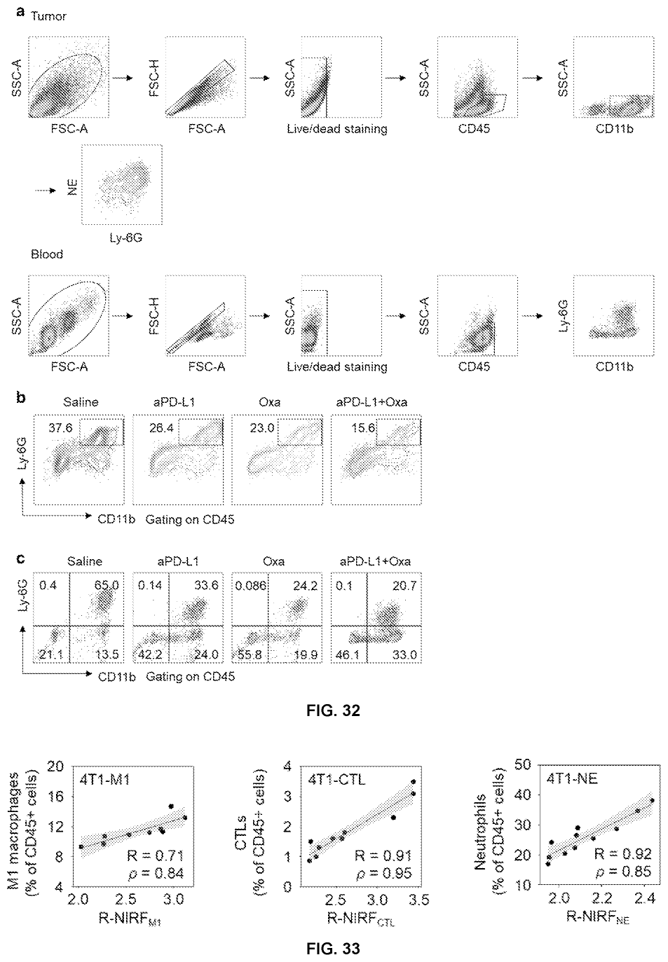

The immunofluorescence staining showed that greater than 75% of red signals of TAMRs well overlapped with green signals of TILs labeled with FITC-tagged antibodies (FIGS. 27-29); besides, the double positive cells defined by those labeled with both TAMRs and antibody accounted for ˜55% of respective TILs (FIGS. 22d and 27-29). Flow cytometry (FIG. 22e) revealed that, the highest level of iNOS⁺Cas-1⁺ cells was found in the tumor of aPD-L1/Oxa treated mice, which was 1.9-, 2.3- and 3.2-fold higher than those of aPD-L1, Oxa and untreated mice, respectively. The highest level of CD8⁺GrB⁺ cells was found in the tumor of aPD-L1/Oxa treated mice, which was 1.6-, 1.6- and 3.4-fold higher than those of aPD-L1, Oxa and untreated mice, respectively. In contrast, the lowest level of Ly-6G⁺NE⁺ cells was detected in the tumor of aPD-L1/Oxa treated mice, which was 1.9-, 1.8- and 2.9-fold lower than those of aPD-L1, Oxa and untreated mice, respectively. Similar trends of leukocytes were also found in the lymph nodes as well as peripheral bloods (FIGS. 30-32). These data confirm that the activation location of TAMRs was specific to TILs and their signals were well correlated with flow cytometry.

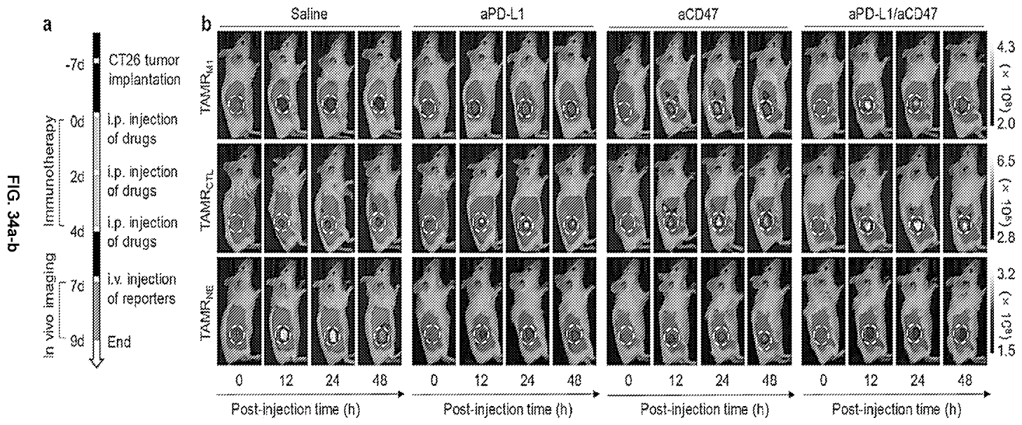

The relationship between TAMRs and TILs was further evaluated with a simple linear regression model. A positive correlation was found between R-NIRF_{M1} and levels of iNOS⁺Cas-1⁺ cells, with a correlation coefficient (R) of 0.75, and a Pearson's r value (p) of 0.82. R-NIRF_{CTL} and R-NIRF_{NE} were positively correlated with the levels of CD8*GrB⁺ cells and Ly-6G⁺NE⁺ cells (R_{CTL}=0.86, ρ_{CTL}=0.93, R_{NE}=0.90 and ρ_{NE}=0.95), respectively (FIG. 22f). In addition, TILs were further profiled as proportions of CD45⁺ cells (all leukocytes), showing that aPD-L1/Oxa treated group had the largest populations of CTLs (5.3%) and M1 macrophages (20.0%), but the smallest populations of neutrophils (15.6%) (FIG. 22g). Moreover, R-NIRFs coincided well with proportions of respective TILs (R>0.70, ρ>0.83) (FIG. 33). These data confirm that the ratiometric imaging signals (R-NIRFs) were valid for non-invasive and real-time profiling TILs in 4T1 tumors.

### Example 8. Real-Time NIRF Imaging of TILs in Highly Immunogenic Tumor

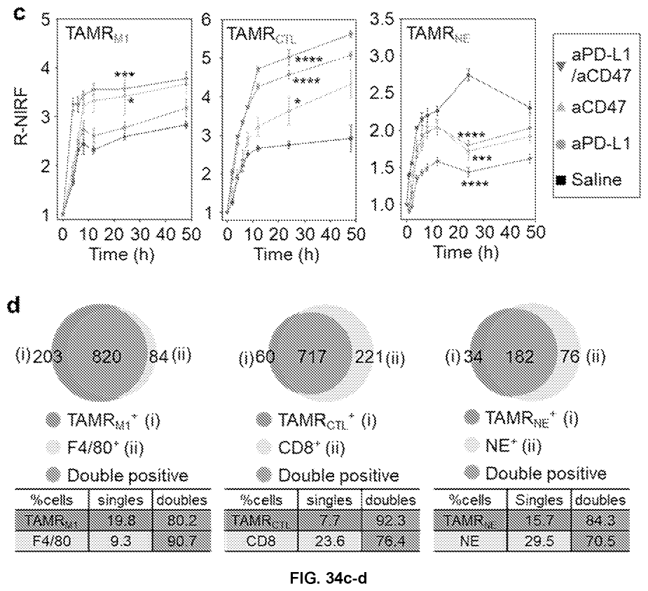

The capabilities of TAMRs (prepared in Example 1) for in vivo real-time imaging of TILs were further evaluated against mice bearing CT26 tumor model, which is considered as a highly immunogenic tumor as it shows a higher presence of tumor-suppressive leukocytes (Taylor, M. A. et al., J. Immunother. Cancer 2019, 7, 328). Mice were administrated with aPD-L1, anti-Cluster of Differentiation 47 (aCD47) or the combination of aPD-L1 and aCD47 (aPD-L1/aCD47) intraperitoneally, followed by systemic administration of TAMRs and PVP-IR800 (FIG. 34a). aCD47 has been widely used to block the interaction between tumor surface CD47 and signal regulatory protein-α (SIRPa) overexpressed on the membrane of myeloid cells, including macrophages, DCs, etc (van den Berg, T. K. & Valerius, T., Nature reviews. Clin. Oncol. 2019, 16, 275-276). The biological studies were carried out by following the protocols in Example 7.

### Results and Discussion

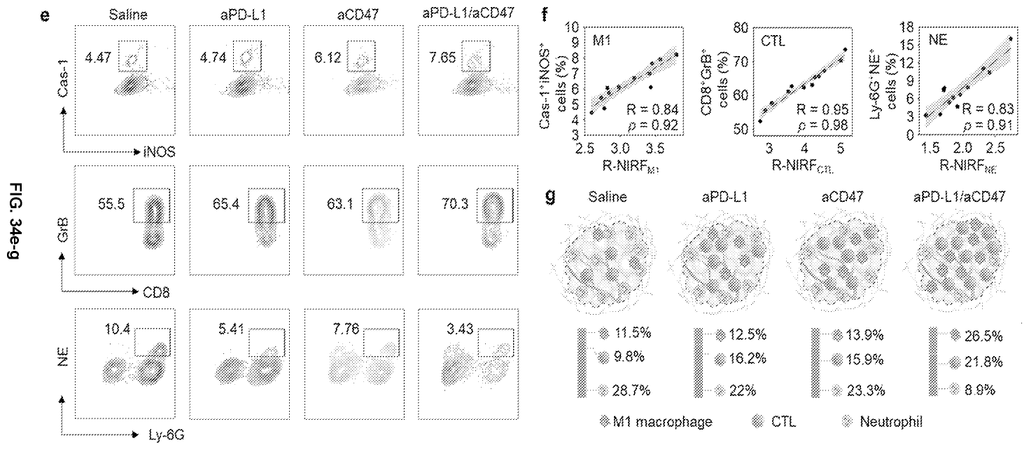

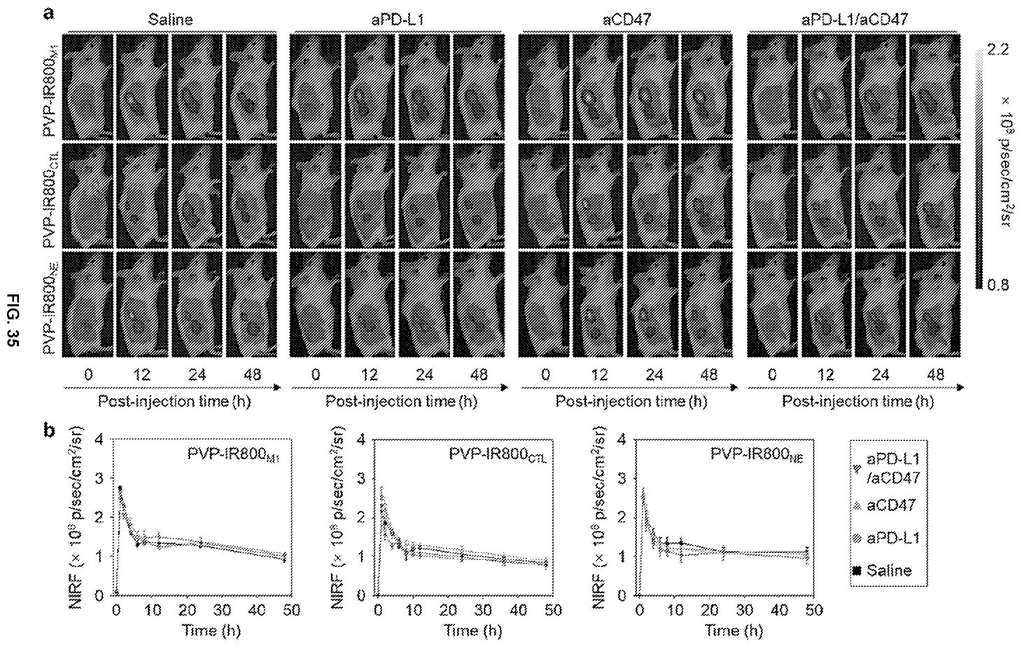

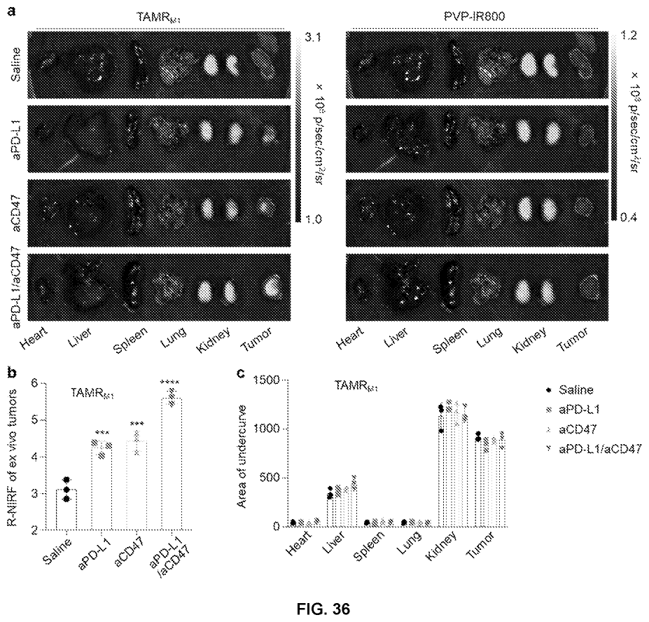

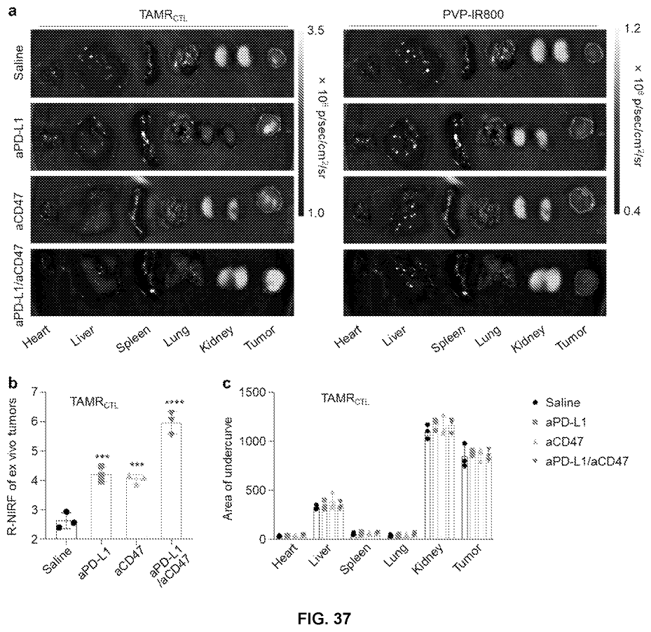

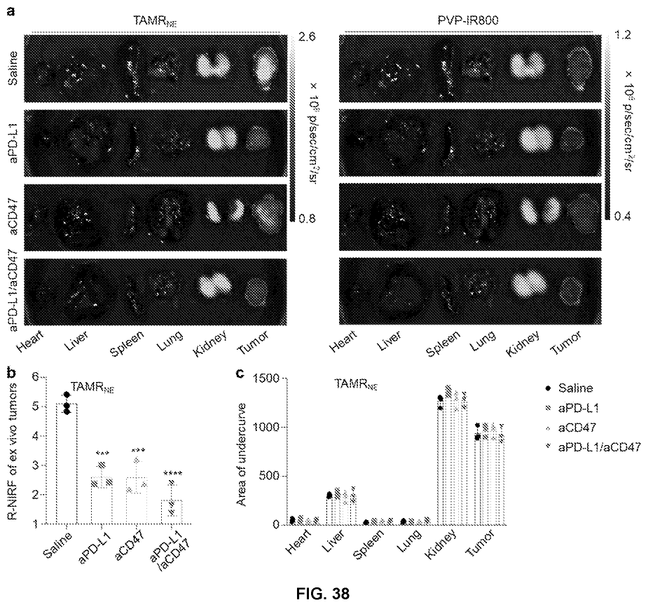

After three times of immunotherapies, TAMRs and the “always-on” reference probe (PVP-IR800) were intravenously co-injected into living mice for longitudinal NIRF imaging (FIG. 34a). NIRF signals of TAMRs and PVP-IR800 in the tumor regions reached maxima at 24 h and 2 h post-injection of reporters, respectively (FIGS. 34b and 35). Thus, the tumor R-NIRFs for different treatment groups were compared at 24 h post-injection of reporters. R-NIRFM1 was the highest for aPD-L1/aCD47 treated mice, which was 1.3-, 1.1- and 1.5-fold higher than those for aPD-L1, aCD47 and untreated mice. R-NIRF_{CTL} was the highest for aPD-L1/aCD47 treated mice, which was 1.1-, 1.4- and 1.9-fold higher than those for aPD-L1, aCD47 and untreated mice. In contrast, R-NIRF_{NE} was the lowest for aPD-L1/aCD47 treated mice, which was 1.3-, 1.2- and 1.9-fold lower than those for aPD-L1, aCD47 and untreated mice (FIGS. 34c and 36-38). These data indicate that TAMR_{M1} and TAMR_{CTL} showed the highest activation, while TAMR_{NE} showed the lowest activation in the tumors of aPD-L1/aCD47 treated mice.

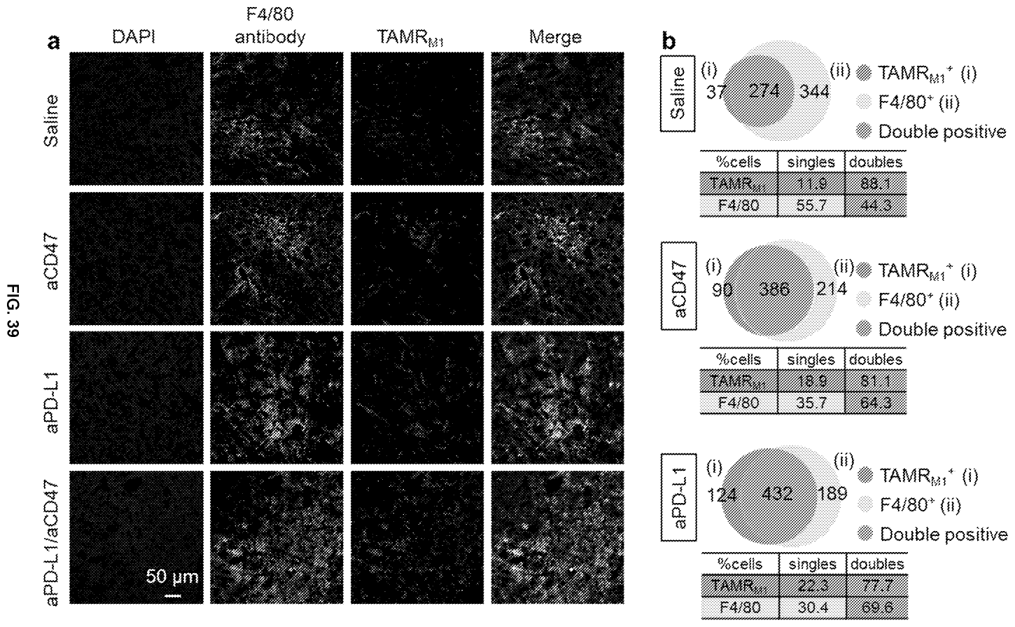

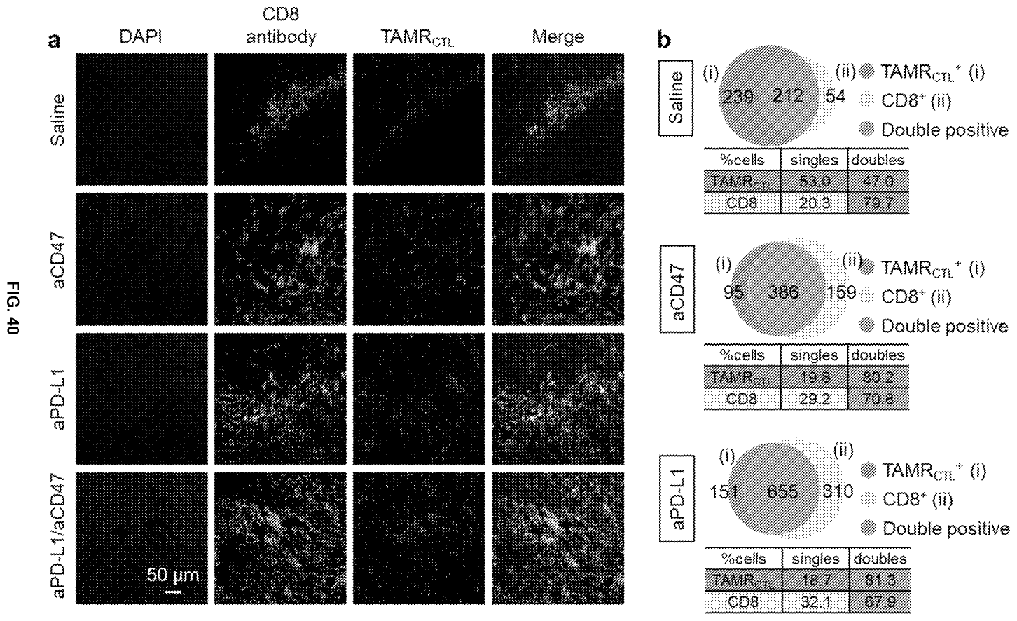

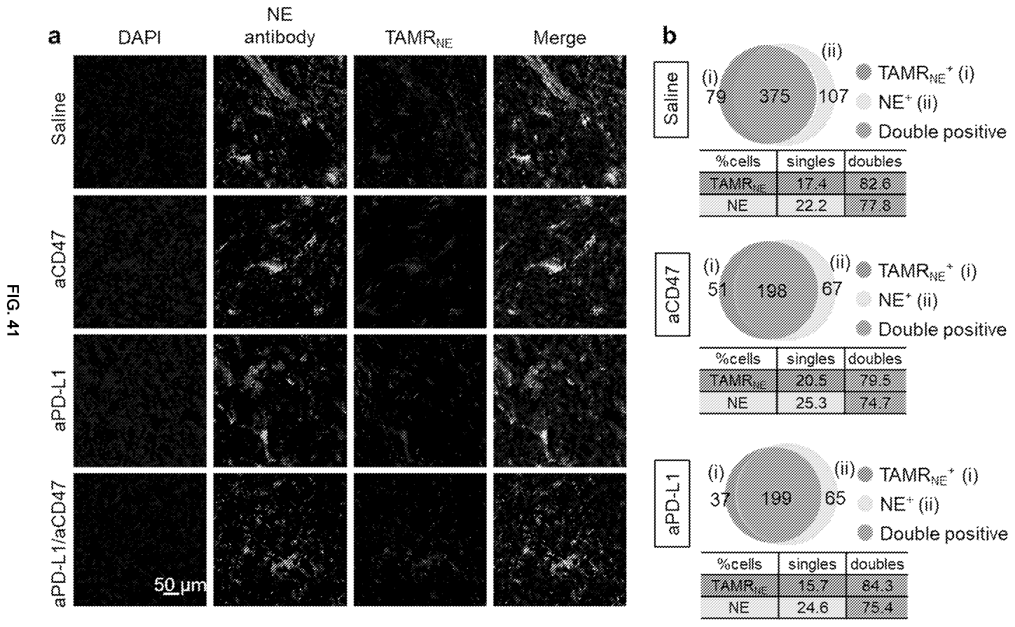

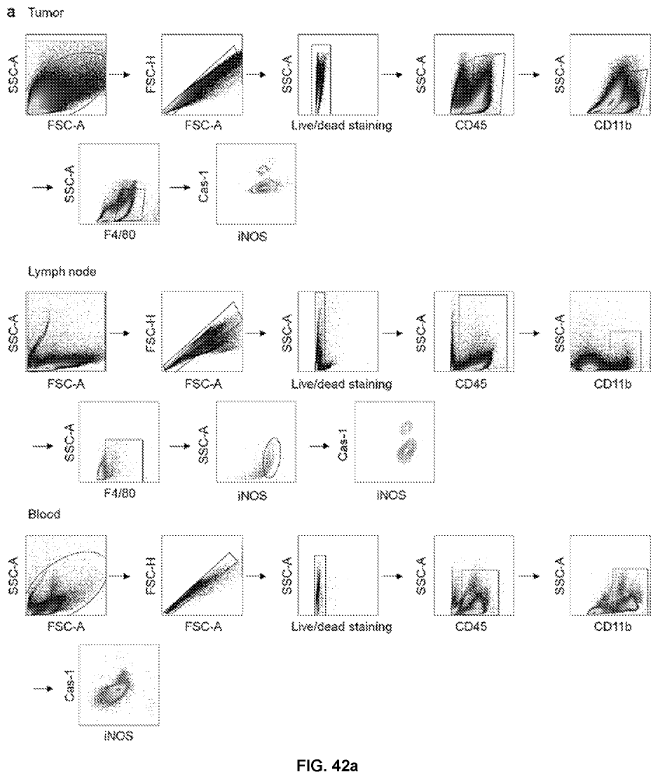

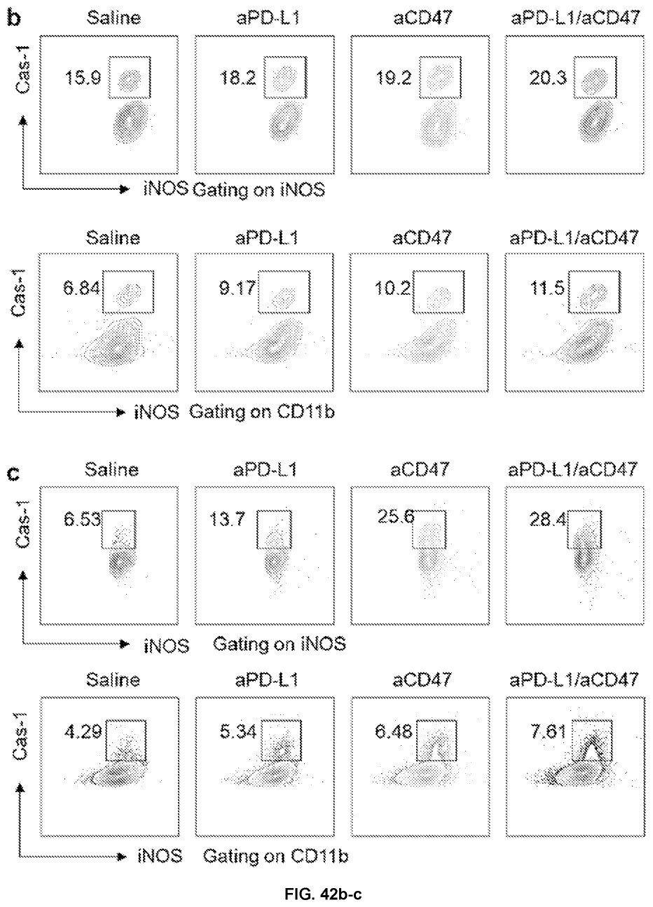

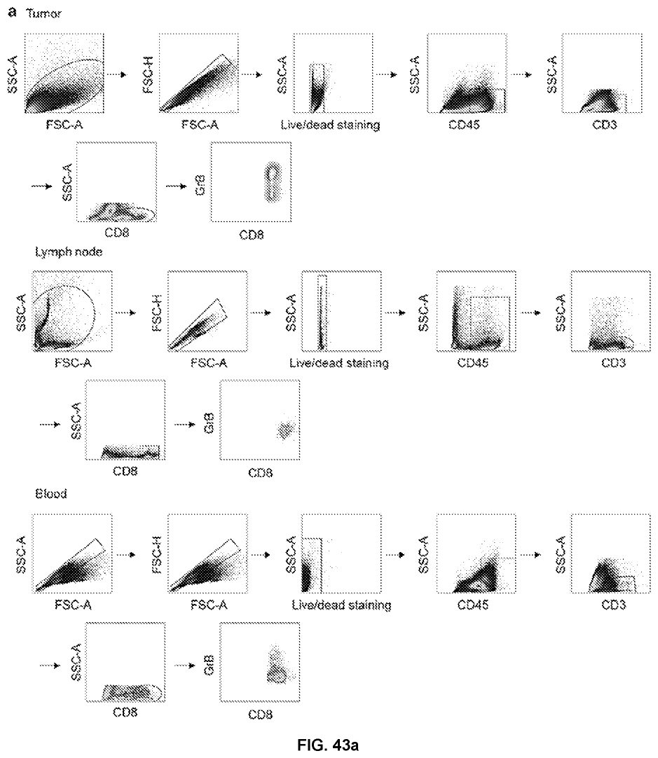

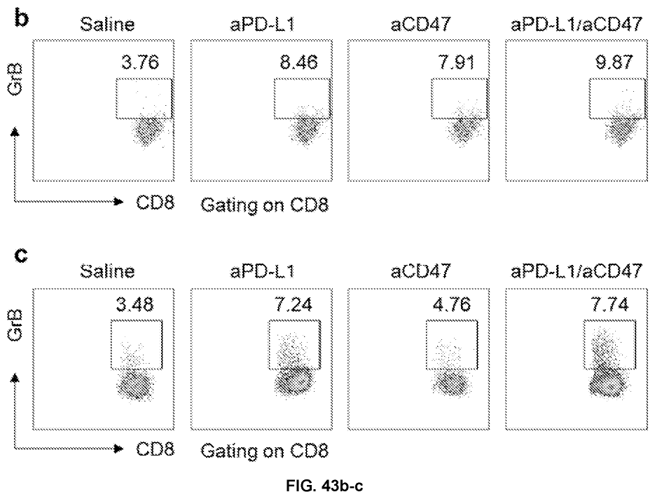

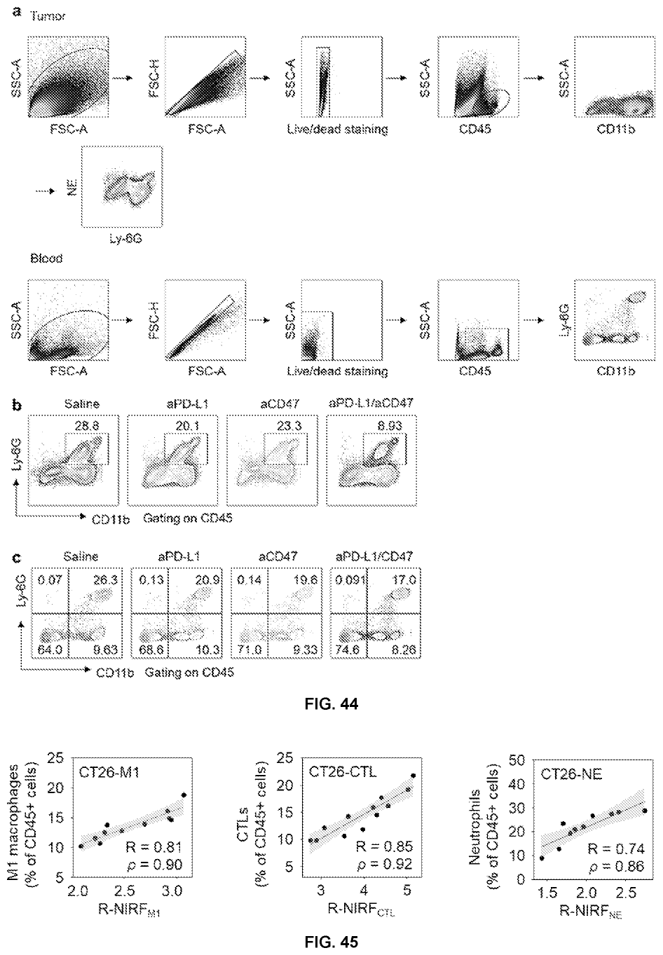

The immunofluorescence staining of tumor sections showed that red signals of TAMRs overlapped well with green signals of TILs labeled with FITC-tagged antibodies (FIGS. 39-41). The double positive cells accounted for 80-92% of TAMRs-localized cells, and 70-91% of respective TILs (FIGS. 34d and 39-41). Flow cytometry analyses showed that the highest level of iNOS⁺Cas-1⁺ cells was observed in the tumor of aPD-L1/aCD47 treated mice, which was 1.6-, 1.3- and 1.7-fold higher than those of aPD-L1, aCD47 and untreated mice, respectively. The highest level of CD8⁺GrB⁺ cells was observed in the tumor of aPD-L1/aCD47 treated mice, which was 1.1-, 1.1- and 1.3-fold higher than those of aPD-L1, aCD47 and untreated mice, respectively, while the lowest level of Ly-6G⁺NE⁺ cells was observed in the tumor of aPD-L1/aCD47 treated mice, which was 1.6-, 2.3- and 3.0-fold lower than those of aPD-L1, aCD47 and untreated mice, respectively (FIG. 34e). Leukocytes in lymph nodes and peripheral bloods exhibited similar trends (FIGS. 42-44). These data confirm that the activation location of TAMRs was specific to TILs and their signals were well correlated with flow cytometry.

The relationship between TAMRs and TILs was further evaluated with a simple linear regression model. R-NIRF_{M1} positively correlated with the levels of iNOS⁺Cas-1⁺ cells in the tumor regions (R_{M1}=0.84, ρ_{M1}=0.91). R-NIRF_{CTL} and R-NIRF_{NE} were also positively correlated with the levels of CD8⁺GrB⁺ cells and Ly-6G⁺NE⁺ cells, respectively (R_{CTL}=0.85, ρ_{CTL}=0.92, R_{NE}=0.88, ρ_{NE}=0.94) (FIG. 34f). In addition, TILs were further profiled as proportions of CD45⁺ cells, showing that aPD-L1/aCD47-treated group had the largest populations of CTLs (21.8%) and M1 macrophages (26.5%), but the smallest populations of neutrophils (8.9%) (FIG. 34g). Moreover, positive correlations between R-NIRFs and the proportions of TILs were observed (FIG. 45). These results further confirm that the ratiometric imaging signals (R-NIRFs) were valid for non-invasive and real-time profiling TILs in CT26 tumors.

Therefore, this array of TAMRs enabled real-time multiplex profiling of TILs in TIME, providing a non-invasive way to accurately map out the intertumoral immune contexture. Moreover, TAMRs had high specificity and sensitivity towards TILs, showing over 70% overlap with their respective TILs in immunofluorescence staining of tumor slices (FIGS. 22d and 34d), and the high correlation of their R-NIRFs (R>0.74, P>0.81) with the levels of TILs measured from flow cytometry (FIGS. 22f and 34f).

### Example 9. Predication of Cancer Immunotherapy

The therapeutic efficacies of various treatments against both 4T1 and CT26 tumor-bearing mice were evaluated.

### Urinalysis of Tumor-Bearing Mice

For urinalysis, both 4T1 and CT26 tumor-bearing mice treated with different immunotherapeutic were intravenously injected with PVP-IR800 (3 μmol/kg mice) and TAMRs (prepared in Example 1, 5 μmol/kg mice), and placed into metabolic cages. At 12 post-injection timepoint, the urine samples were collected and imaged by IVIS spectrum imaging system with excitation at 675 nm and emission at 720 nm for monitoring of activated TAMRs, and excitation at 745 nm and emission at 800 nm for monitoring of IR800.

### Tumor Control Rate

The tumor control rate was calculated with the following formulas:

$$\begin{matrix}Tumor growth rate ( TGR \% ) = Vt / {Vt}_{0} \times 100 \% , \\ Tumor control rate ( \% ) = [ ( T G {R}_{C} - T G {R}_{S} ) / {TGR}_{C} ] \times 100 \% ,\end{matrix}$$

Vt₀ means the volume of tumor on day 0, Vt means the volume at certain time, c means the control group (saline, without irradiation), and s means the group needed calculation.

### Whole-Slide Imaging Analysis of TAMRs Distribution

Both 4T1 and CT26 tumor-bearing mice treated with different treatments were intravenously injected with TAMRs (prepared in Example 1, 5 μmol/kg). After 24 h post-injection, mice were euthanized, and tumors were collected, followed by fixation in 4% PFA. After being dehydrated with 30% sucrose, tumor tissues were embedded in O.C.T. medium and cut into 10-μm sections. Tumor sections were washed with PBST, following by staining the nucleus with DAPI. The fluorescence images of the tumor sections were captured on LSM800 (Zeiss) using the tiles mode.

### ROC

We have inputted in vivo fluorescence imaging data (R-NIRF) at 24 h (n=3) and 48 h (n=3) of each group into GraphPad data table, and plotted it using ROC cure model in Column analyses of GraphPad. Combination of multiple TAMRs was derived from logistic regression of R-NIRFs of TAMR_{M1}, TAMR_{CTL} and TAMR_{NE}.

### PCA

We have inputted in vivo fluorescence imaging data at 24 h (n=3) and 48 h (n=3), and urinary data at 12 h (n=3) of each group into origin workbook, and created PCA figure using an enhanced version of Principal Component Analysis tool of origin.

### Results and Discussion

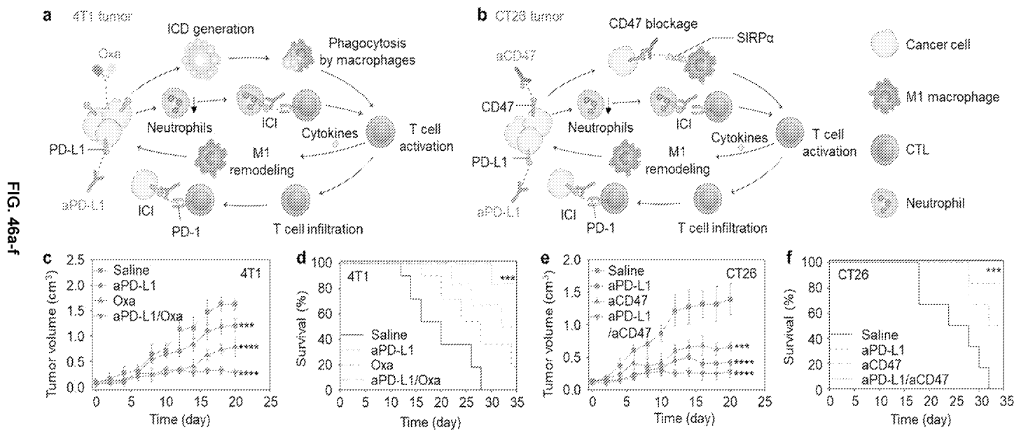

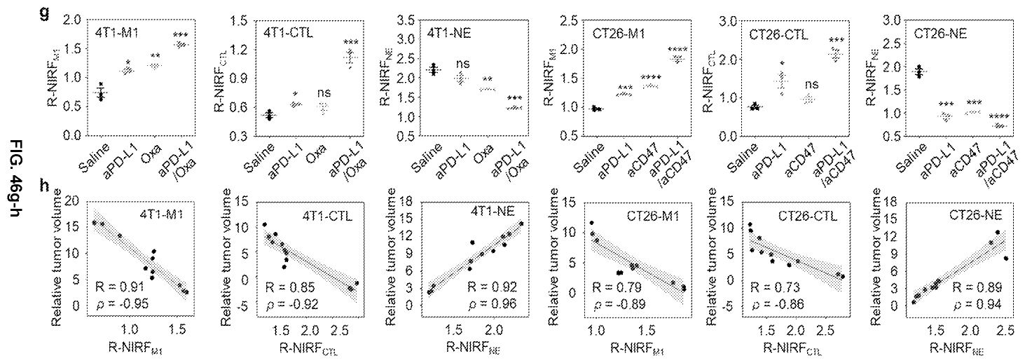

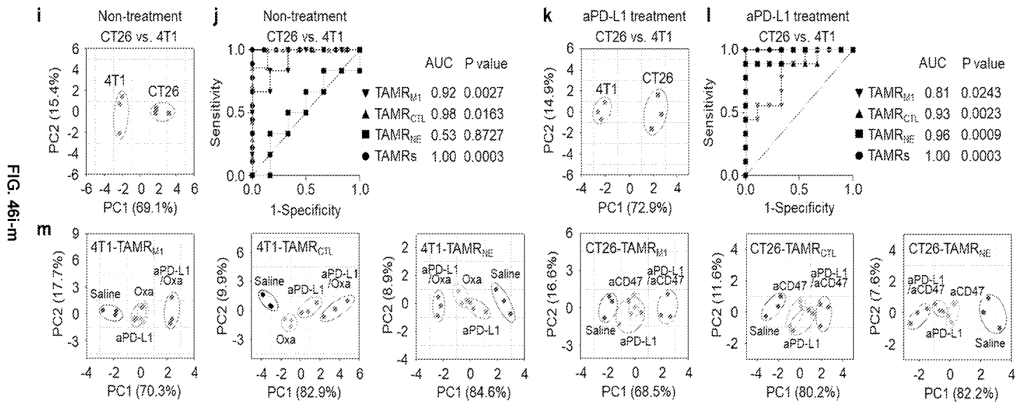

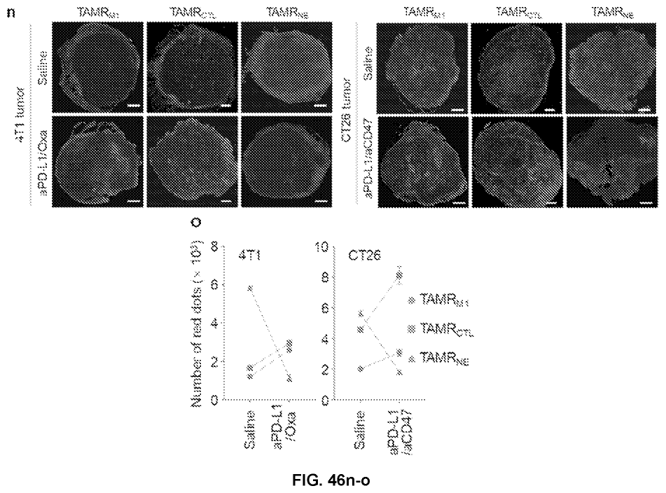

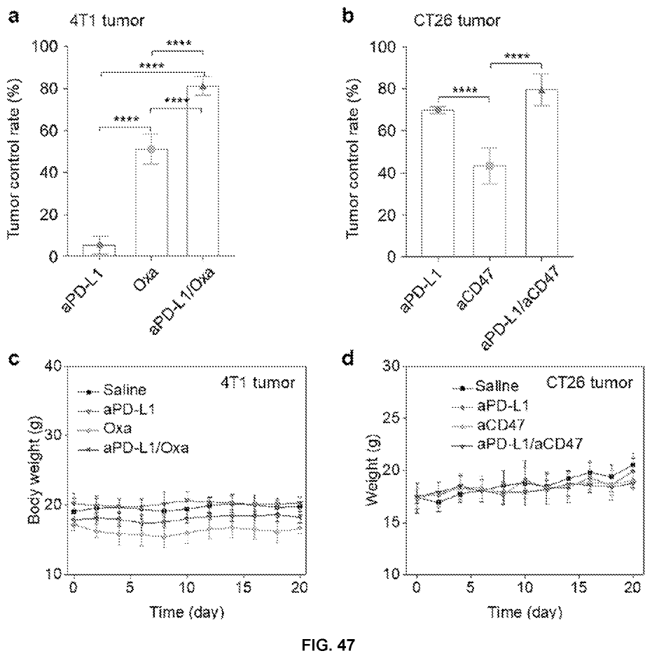

For 4T1 tumor-bearing mice, the highest tumor control rate (TCR) (85%) was observed for aPD-L1/Oxa treatment, which was 9.9- and 1.8- and fold higher than that of aPD-L1 (8.6%) and Oxa (48%) treatment, respectively (FIGS. 46c and 47). In addition, survival rate of the mice treated with aPD-L1/Oxa reached 83%, significantly higher than that of aPD-L1 (17%) and Oxa (50%). Note that all untreated mice died at day 28 (FIG. 46d). The high therapeutic efficacy of aPD-L1/Oxa treatment was attributed to the synergetic effect: Oxa induced ICD generation and promoted antigen presentation to awake CTLs. Upon CTLs recruitment to the TIME, aPD-L1 inhibited the association of PD-L1 with PD-1 and favored the cytotoxicity of CTLs against cancer cells. Concurrently, activated CTLs generated cytotoxic cytokines which further promoted M1 macrophages polarization, and inhibited the immunosuppressive resident neutrophils, leading to effective killing of cancer cells (FIG. 46a). For CT26 tumor-bearing mice, the highest TCR (82%) was observed in aPD-L1/aCD47 treatment, which was 1.2- and 1.3-fold higher than that of aPD-L1 (71%) and aCD47 (62%), respectively (FIGS. 46e and 47). Survival rate of the mice treated with aPD-L1/aCD47 reached 100%, higher than that of aPD-L1 (83%) and aCD47 (50%). Note that all untreated mice died at day 32 (FIG. 46f).

The high therapeutic efficacy of aPD-L1/aCD47 treatment was attributed to the synergetic effect: aCD47 inhibited CD47-SIRPa interaction, and promoted phagocytosis of apoptotic cells by macrophages, which further enhanced priming of CTLs. Concurrently, aPD-L1 favored the cytotoxicity of CTLs, and provided an immunostimulatory microenvironment which suppressed the functions of immunosuppressive neutrophils, resulting in an enhanced anticancer efficacy (FIG. 46b).

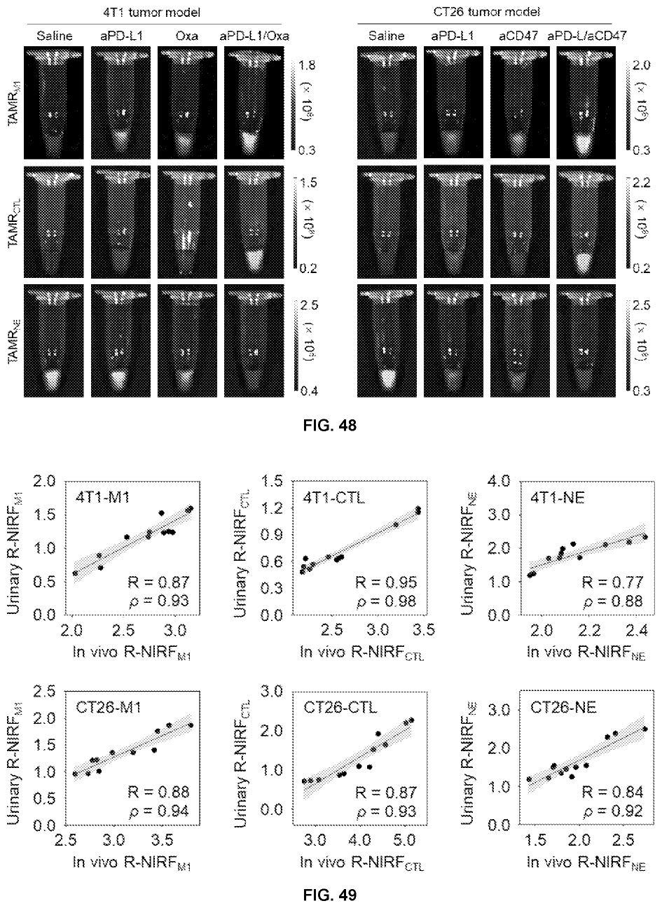

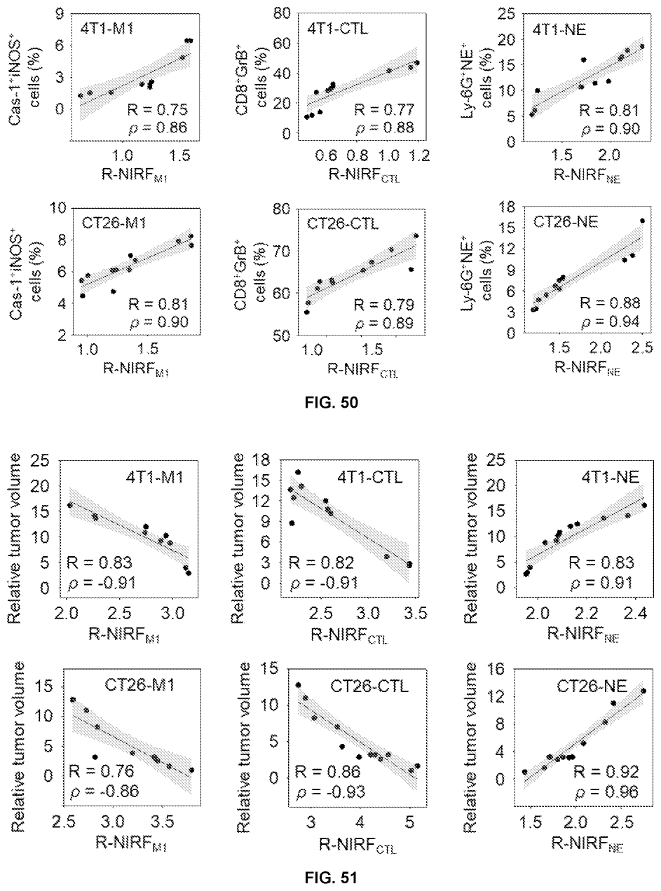

High renal clearance efficiency and fluorescence turn-on response of TAMRs provide a convenient way for fluorescence urinalysis of TILs, making TAMRs highly promising for clinical translation. To evaluate the potential of TAMRs for urinalysis, urine samples of mice post-injection of reporters were collected for NIRF measurement. In general, the urinary signals coincided well with the real-time imaging data: urinary R-NIRF_{M1} and R-NIRF_{CTL} were the highest while R-NIRFNE was the lowest for aPD-L1/Oxa treated 4T1 tumor-bearing mice; urinary R-NIRF_{M1} and R-NIRF_{CTL} were the highest while R-NIRF_{NE} was the lowest for aPD-L1/aCD47 treated CT26 tumor-bearing mice. (FIGS. 46g and 48-49). All urinary R-NIRFs accurately reflected populations of their respective TILs (FIG. 50). Consistent with the real-time imaging data, urinary R-NIRFs of excreted TAMRs accurately reflected their respective TILs populations (R>0.74, ρ>0.85) (FIG. 50).

To determine whether the urinary R-NIRFs at day 7 could predict the therapeutic outcomes at day 20, the correlation analysis between R-NIRFs and the relative tumor volumes was performed. Consistent with the in vivo ratiometric imaging data (FIG. 51), urinary R-NIRF_{M1} was negatively correlated with the relative tumor volumes with a correlation coefficient (R) of 0.91 and 0.79, and Pearson's r value (p) of −0.95 and −0.89 for 4T1 and CT26 tumor-bearing mice, respectively. In addition, urinary R-NIRF_{CTL} was negatively correlated with the relative tumor volumes (4T1 model: R_{CTL}=0.85, pc_{TL}=−0.92; CT26 model: R_{CTL}=0.73, pc_{TL}=−0.86) (FIG. 46h). In contrast, urinary R-NIRFNE was positively correlated with the relative tumor volumes (4T1 model: R_{NE}=0.92, p_{NE}=0.96; CT26 model: R_{NE}=0.89, p_{NE}=0.94). This indicates that M1 macrophages and CTLs were positive prognostic indicators while neutrophils were negative prognostic indicators for therapeutic outcomes, which are consistent with previous clinical findings (Edin, S. et al., PLOS ONE 2012, 7, e47045; Galon, J. et al., Science 2006, 313, 1960-1964; and Kaneko, M. et al., Oncology 2012, 82, 261-268).

Because urinalysis was performed at day 7 which was two weeks earlier than the treatment endpoint, urinary R-NIRFs of TAMRs served as a non-invasive and accurate way to predict therapeutic outcomes. In addition, comparison of the urinary R-NIRFs for different tumors revealed that positive prognostic TAMR_{M1} and TAMRC_{TL} exhibited 1.3- and 1.5-fold higher R-NIRFs in CT26 tumors than that in 4T1 tumors, respectively. Thus, TAMR_{M1} and TAMRC_{TL} clearly distinguished poorly immunogenic tumor (4T1) from highly immunogenic tumor (CT26), showing their stratification capability.

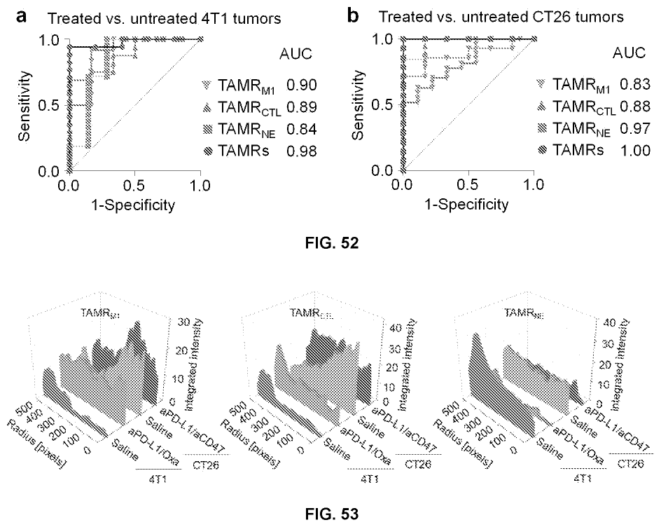

The ability of TAMRs in companion diagnosis and prediction of cancer immunotherapy was further evaluated by PCA. PCA of R-NIRFs of TAMRs distinctly separated untreated 4T1 and CT26 tumors (FIG. 46i). ROC analysis of TAMRs against different tumor models revealed an AUC of 0.92, 0.98 and 0.53 for TAMR_{M1}, TAMRC_{TL} and TAMR_{NE}, respectively, while the combination of TAMRs better differentiated 4T1 from CT26 tumors (AUC=1.00) (FIG. 46j). PCA of R-NIRFs of TAMRs further significantly separated aPD-L1 treated 4T1 and CT26 tumors (FIG. 46k). ROC analysis of TAMRs against aPD-L1 treated 4T1 and CT26 tumors revealed an AUC of 0.81, 0.93 and 0.96 for TAMR_{M1}, TAMRC_{TL} and TAMR_{NE}, respectively, and the combination of TAMRs better differentiated aPD-L1 treated 4T1 from aPD-L1 treated CT26 tumors (AUC=1.00) (FIG. 46l). This result is consistent with previous data that CT26 tumors showed effective responses to ICI, while 4T1 tumors hardly showed responses to ICI (Mosely, S. I. et al., Cancer Immunol. Res. 2017, 5, 29-41). Thus, TAMR-based multiplex urinalysis allows the distinction of untreated CT26 tumors from untreated 4T1 tumors, and aPD-L1 treated CT26 tumors from aPD-L1 treated 4T1 tumors with high accuracy (AUC=1.00) (FIG. 46i-l). Moreover, urinary R-NIRFs of TAMRs at the early stage of cancer immunotherapies predicted the relative tumor volumes at the endpoints, wherein TAMR_{M1} and TAMRC_{TL} had negative correlation (R>0.72, |ρ|>0.85), and TAMR_{NE} had positive correlation to the relative tumor volumes (R>0.88, ρ>0.93) (FIG. 46h). PCA of TAMRs further significantly distinguished tumor-bearing mice in different treatment groups (FIG. 46m). In addition, ROC analysis of the combination of multiple TAMRs distinguished untreated tumors from treated tumors with high accuracy (AUC_{4T1}=0.98, AUC_{CT26}=1.00) (FIG. 52). These data demonstrate the potential of TAMRs in stratification of patients and evaluation of immunotherapeutic outcome.

TAMRs could also be applied for the microscopic examination of whole-tumor sections, which is one of clinical approaches for evaluation of TILs (FIGS. 46n,o). For 4T1 tumor sections, relative to initial location at the periphery of untreated ones, 2- and 1.8-fold higher densities of TAMR_{M1} and TAMR_{CTL} were observed to diffuse to the center for aPD-L1/Oxa treated sections, respectively (FIG. 53); whereas, 4.5-fold lower density of TAMRNE was left at the periphery for aPD-L1/Oxa treated sections, relative to untreated ones. For CT26 tumor sections, relative to untreated tumor sections, 1.5- and 1.7-fold higher densities of TAMR_{M1} and TAMR_{CTL} were observed to non-homogeneously distribute inside aPD-L1/aCD47 treated sections, respectively; whereas, 3.1-fold lower density of TAMR_{NE} was left inside aPD-L1/aCD47 treated tumor sections. Because over 75% of TAMRs co-localized with TILs as confirmed by immunofluorescence staining in Example 7 (FIGS. 22d and 34d), TAMRs in the tumors represented the landscape of their respective TILs. In addition, untreated 4T1 and CT26 tumors presented totally different immunospatial profiles, which is consistent with previous findings (Taylor, M. A. et al., J. Immunother. Cancer 2019, 7, 328) that highly immunogenic (CT26) tumors had high tumor-suppressive leukocytes infiltration, while poorly immunogenic (4T1) tumors had low tumor-suppressive leukocytes infiltration. Thus, TAMRs allows one to accurately distinguish poorly immunogenic (4T1) from highly immunogenic (CT26) tumors, and closely monitor the infiltration of CTLs, polarization of macrophages, and variation of neutrophils along with different therapies for predication of therapeutic outcomes.

Thus, in addition to urinalysis, TAMRs was able to delineate the spatial distribution of TILs in whole-tumor sections via microscopic examination, serving as another clinical utility for stratification and evaluation of cancer therapy. TAMRs staining revealed the changes in the location and density of TILs after therapy, showing more positive prognostic TILs (M1 macrophages and CTLs) and less negative prognostic TILs (neutrophils) distributed in the center of tumor, and 1.5 to 2.0-fold higher levels of M1 macrophages, 1.6 to 1.9-fold higher levels of CTLs and 3.1 to 4.5-fold lower levels of neutrophils than untreated tumors (FIGS. 46n,o).

In summary, we developed an unprecedented set of TILs-specific molecular fluorescence reporters (TAMRs) for companion diagnosis and prognosis of cancer immunotherapy. TAMRs have a unique dual-locked sensing mechanism, permitting specific fluorescence correlation with TILs. TAMR-based real-time imaging and urinalysis are non-invasive and dynamic but competent to profile multiple TILs with the sensitivity and specificity at the level equal to static flow cytometry analysis and invasive biopsy. The signal correlation of TAMRs allows for accurate analyses of tumor immunogenicity and longitudinal monitoring of changes in TIME.

Thus, TAMRs not only present a high-throughput, non-invasive, and effective way to screen combinational immunotherapeutic agents in preclinical settings, but also hold the potential in clinical settings to stratify patients for personalized combinational cancer immunotherapy, optimize immunotherapeutic intervention, and predict immunotherapeutic outcome. The modular dual-locked tandem design of TAMRs can be generalized for specific detection of biomarkers from the targeted cell at targeted disease site, advancing the way for precision biomarker profiling using molecular probes.

1. A compound of formula I: 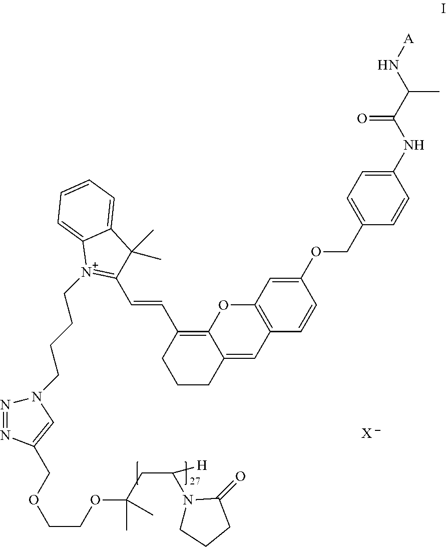
wherein:
X⁻ represents a counterion; and
A represents an amino acid moiety that is cleavable by an enzyme associated with a leukocyte, or a pharmaceutically acceptable salt or solvate thereof.

2. The compound according to claim 1, or a salt or solvate thereof, wherein A is selected from: 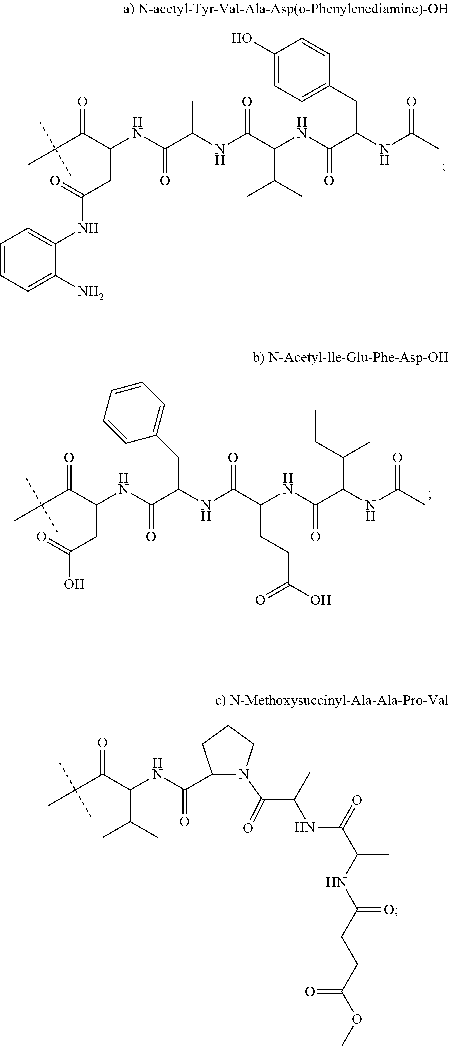
where the point of attachment is denoted by the dotted line.

3.-6. (canceled)

7. A method of diagnosing a condition or disease in a tissue and/or an organ, the method comprising the steps of administering a compound as defined in claim 1, or a salt or solvate thereof to a subject in need of diagnosis and determining the presence or absence of a condition or disease in a tissue and/or an organ using near-infrared fluorescence.

8. A method of determining the susceptibility of a tumour tissue to an immunotherapy, the method comprising the steps of:
(i) subjecting a tumour tissue to a desired course of immunotherapy;
(ii) administering a compound as defined in claim 1, or a salt or solvate thereof to the tumour tissue along with an always on reference reporter compound;
(iii) after a period of time determining the ratio of near-infrared fluorescence obtained from a metabolite of the compound as defined in claim 1 relative to the always on reference reporter compound; and
(iv) determining the susceptibility of the tumour tissue to the immunotherapy based on the ratio obtained in step (iii).

9. The method according to claim 8, wherein the method is conducted in vivo.

10. The method according to claim 8, wherein the method is conducted in vitro.

11. The method according to claim 8, wherein the method further involves subjecting a subject to a therapeutic treatment regimen based on the results obtained by the method.

12. The method according to claim 8, wherein the always on reference reporter compound is: [chemical structure]
